(19) United States
(12) Patent Application Publication
(10) Pub. No.: US 2015/0125452 A1
(43) Pub. Date: May 7, 2015

(54) COMBINATION TREATMENTS COMPRISING C-MET ANTAGONISTS AND B-RAF ANTAGONISTS

(71) Applicant: GENENTECH, INC., SOUTH SAN FRANSCICO, CA (US)

(72) Inventors: Timothy R. Wilson, San Mateo, CA (US); Hartmut Koeppen, San Mateo, CA (US); Mark Merchant, Belmont, CA (US); Jeffrey Settleman, Mill Valley, CA (US)

(21) Appl. No.: 14/535,071

(22) Filed: Nov. 6, 2014

Related U.S. Application Data
(63) Continuation of application No. 13/622,878, filed on Sep. 19, 2012, now abandoned.
(60) Provisional application No. 61/536,436, filed on Sep. 19, 2011.
(60) Provisional application No. 61/551,328, filed on Oct. 25, 2011.
(60) Provisional application No. 61/598,783, filed on Feb. 14, 2012.
(60) Provisional application No. 61/641,139, filed on May 1, 2012.

(51) Int. Cl. A61K 39/395; G01N 33/574; C12Q 1/68; A61K 31/437; A61K 31/4545

(52) U.S. Cl. CPC A61K 39/3955; A61K 31/437; A61K 31/4545; C12Q 1/6886; G01N 33/5748; G01N 33/57496; G01N 33/57488; G01N 2800/52; C12Q 2600/106; C12Q 2600/158
USPC 424/138.1; 514/300; 435/6.14; 435/6.12; 435/6.11; 435/7.23; 435/7.4; 506/9; 506/2; 435/7.92; 435/7.1

(57) ABSTRACT

The present invention relates generally to the fields of molecular biology and growth factor regulation. More specifically, the invention relates to therapies for the treatment of pathological conditions, such as cancer.

### CROSS-REFERENCE TO RELATED APPLICATIONS

This application is a continuation of U.S. patent application Ser. No. 13/622,878, filed Sep. 19, 2012, which claims priority to U.S. patent application No. 61/536,436, filed Sep. 19, 2011, U.S. patent application No. 61/551,328, filed Oct. 25, 2011, U.S. patent application No. 61/598,783, filed Feb. 14, 2012, and U.S. patent application No. 61/641,139, filed May 1, 2012, which are incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Feb. 18, 2014, is named P47361R1D1US_Sequence_Listing.txt and is 16,493 bytes in size.

### FIELD OF THE INVENTION

The present invention relates generally to the fields of molecular biology and growth factor regulation. More specifically, the invention relates to therapies for the treatment of pathological conditions, such as cancer.

### BACKGROUND

Cancer remains to be one of the most deadly threats to human health. In the U.S., cancer affects nearly 1.3 million new patients each year, and is the second leading cause of death after heart disease, accounting for approximately 1 in 4 deaths. For example, breast cancer is the second most common form of cancer and the second leading cancer killer among American women. It is also predicted that cancer may surpass cardiovascular diseases as the number one cause of death within 5 years. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Despite the significant advancement in the treatment of cancer, improved therapies are still being sought.

All references cited herein, including patent applications and publications, are incorporated by reference in their entirety.

### SUMMARY OF THE INVENTION

Uses of a c-met antagonist for effectively treating cancer patients are provided. This application also provides better methods for diagnosing disease for use in treating the disease optionally with c-met antagonist in combination with a B-raf antagonist. In particular, results are described demonstrating that combination treatment using B-raf antagonist vemurafenib (PLX-4032) and c-met antagonist resulted in a statistically significant improvement in tumor regression, including a striking improved in partial responses, compared to treatment with vemurafenib alone. C-met expression was inversely correlated with sensitivity to vemurafenib treatment. In addition, patients with B-raf mutant melanoma who had higher levels of circulating hepatocyte growth factor (HGF) showed substantially reduced progression free survival and overall survival when treated with B-raf antagonist, relative to patients with lower circulating HGF levels treated with B-raf antagonist.

The present invention provides combination therapies for treating a pathological condition, such as cancer, wherein a c-met antagonist is combined with a B-raf antagonist, thereby providing significant anti-tumor activity.

In one aspect, provided are methods for treating a cancer patient who has increased likelihood of developing resistance to B-raf antagonist comprising administering an effective amount (in combination) of B-raf antagonist and c-met antagonist.

In one aspect, provided are methods for increasing and/or restoring sensitivity to B-raf antagonist comprising administering to a cancer patient an effective amount of B-raf antagonist and c-met antagonist.

In one aspect, provided are methods for extending period of B-raf antagonist sensitivity comprising administering to a cancer patient an effective amount of B-raf antagonist and c-met antagonist.

In one aspect, provided are methods for treating a patient with B-raf resistant (B-raf antagonist resistant) cancer comprising administering an effective amount of B-raf antagonist and c-met antagonist.

In one aspect, provided are methods for extending duration of response to B-raf antagonist comprising administering an effect amount of B-raf antagonist and c-met antagonist.

In one aspect, provided are methods for delaying or preventing development of HGF-mediated B-raf resistant cancer comprising administering an effective amount of B-raf antagonist and c-met antagonist.

In one aspect, methods are provided for determining c-met biomarker expression, comprising the step of determining whether a patient's cancer expresses c-met biomarker, wherein c-met biomarker expression indicates that the patient is likely to have B-raf antagonist resistant cancer. In some embodiments, the patient's cancer has been shown to express B-raf biomarker. In some embodiments, c-met biomarker expression is protein expression and is determined in a sample from the patient using IHC. In some embodiments, high amount of c-met biomarker (e.g., as determined using c-met IHC or detection of HGF using, e.g., ELISA or IHC) indicates that the patient is likely to have B-raf antagonist resistant cancer. As used herein, “elevated” or “high” c-met refers to an amount of c-met associated with patient responsiveness to a treatment. In some embodiments, low amount of c-met biomarker (e.g., as determined using c-met IHC or detection of HGF using, e.g., ELISA or IHC) indicates that the patient is unlikely to have B-raf antagonist resistant cancer. In some embodiments, high c-met is low, moderate or high c-met expression determined, e.g., relative to c-met staining intensity of control cell pellets A549, H441, H1155, and HEK-293 as described herein. In some embodiments, high c-met is moderate or high c-met expression determined, e.g., relative to c-met staining intensity of control cell pellets A549, H441, H1155, and HEK-293 as described herein. As used herein, a “low” amount of c-met refers to an amount of c-met associated with lack of response to a treatment, or, in some embodiments, an amount of c-met associated with worse response to a treatment (e.g. decreased clinical benefit compared to no treatment). In some embodiments, “low” c-met is low or no c-met expression determined, e.g., relative to c-met staining intensity of control cell pellets A549, H441, H1155, and HEK-293 as described herein. In some embodiments, “low” c-met expression is no c-met expression determined, e.g., relative to c-met staining intensity of control cell pellets A549, H441, H1155, and HEK-293 as described herein.

In one aspect, methods are provided for determining c-met biomarker expression, comprising the step of determining whether a patient's cancer expresses c-met biomarker, wherein c-met biomarker expression indicates that the patient is likely to develop B-raf resistant cancer. In some embodiments, the patient's cancer has been shown to express B-raf biomarker. In some embodiments, c-met biomarker expression is protein expression and is determined in a sample from the patient using IHC. In some embodiments, the patient is treated with B-raf antagonist and c-met antagonist. In some embodiments, high amount of c-met biomarker (e.g., as determined using c-met IHC or detection of HGF using, e.g., ELISA or IHC) indicates that the patient is likely to have B-raf antagonist resistant cancer. In some embodiments, high c-met is low, moderate or high c-met expression determined, e.g., relative to c-met staining intensity of control cell pellets A549, H441, H1155, and HEK-293 as described herein. In some embodiments, high c-met is moderate or high c-met expression determined, e.g., relative to c-met staining intensity of control cell pellets A549, H441, H1155, and HEK-293 as described herein. In some embodiments, “low” c-met is low or no c-met expression determined, e.g., relative to c-met staining intensity of control cell pellets A549, H441, H1155, and HEK-293 as described herein. In some embodiments, “low” c-met expression is no c-met expression determined, e.g., relative to c-met staining intensity of control cell pellets A549, H441, H1155, and HEK-293 as described herein.

In one aspect, methods are provided for determining c-met biomarker expression, comprising the step of determining whether a patient's cancer expresses c-met biomarker, wherein c-met biomarker expression indicates that the patient is a candidate for treatment with c-met antagonist and B-raf antagonist: to increase sensitivity of the patient's cancer to B-raf antagonist, restore sensitivity of the patient's cancer to B-raf antagonist, to extend the period of sensitivity of the patient's cancer to B-raf antagonist, and/or to prevent development of HGF-mediated B-raf antagonist resistance in the patient's cancer. In some embodiments, the patient's cancer has been shown to express B-raf biomarker. In some embodiments, c-met biomarker expression is protein expression and is determined in a sample from the patient using IHC. In some embodiments, the patient is treated with B-raf antagonist and c-met antagonist. In some embodiments, high amount of c-met biomarker (e.g., as determined using c-met IHC or detection of HGF using, e.g., ELISA or IHC) indicates that the patient is likely to have B-raf antagonist resistant cancer. In some embodiments, high c-met is low, moderate or high c-met expression determined, e.g., relative to c-met staining intensity of control cell pellets A549, H441, H1155, and HEK-293 as described herein. In some embodiments, high c-met is moderate or high c-met expression determined, e.g., relative to c-met staining intensity of control cell pellets A549, H441, H1155, and HEK-293 as described herein. In some embodiments, “low” c-met is low or no c-met expression determined, e.g., relative to c-met staining intensity of control cell pellets A549, H441, H1155, and HEK-293 as described herein. In some embodiments, “low” c-met expression is no c-met expression determined, e.g., relative to c-met staining intensity of control cell pellets A549, H441, H1155, and HEK-293 as described herein.

In one aspect, methods are provided for selecting a therapy for a patient with cancer which has been shown to express B-raf biomarker comprising determining expression of c-met biomarker in a sample from the patient, and selecting a cancer medicament based on the level of expression of the biomarker. In some embodiments, the patient is selected for treatment with a c-met antagonist in combination with B-raf antagonist if the cancer sample expresses c-met biomarker. In some embodiments, the patient is treated for cancer using therapeutically effective amount of the c-met antagonist and B-raf antagonist. In some embodiments, the patient is selected for treatment with a cancer medicament other than c-met antagonist if the cancer sample expresses substantially undetectable levels of the c-met biomarker. In some embodiments, high amount of c-met biomarker (e.g., as determined using c-met IHC or detection of HGF using, e.g., ELISA or IHC) indicates that the patient is likely to have B-raf antagonist resistant cancer. In some embodiments, high c-met is low, moderate or high c-met expression determined, e.g., relative to c-met staining intensity of control cell pellets A549, H441, H1155, and HEK-293 as described herein. In some embodiments, high c-met is moderate or high c-met expression determined, e.g., relative to c-met staining intensity of control cell pellets A549, H441, H1155, and HEK-293 as described herein. In some embodiments, “low” c-met is low or no c-met expression determined, e.g., relative to c-met staining intensity of control cell pellets A549, H441, H1155, and HEK-293 as described herein. In some embodiments, “low” c-met expression is no c-met expression determined, e.g., relative to c-met staining intensity of control cell pellets A549, H441, H1155, and HEK-293 as described herein.

In one aspect, methods are provided for identifying a patient as a candidate for treatment with a B-raf antagonist and a c-met antagonist, comprising determining that the patient's cancer expresses c-met biomarker. In some embodiments, high amount of c-met biomarker (e.g., as determined using c-met IHC or detection of HGF using, e.g., ELISA or IHC) indicates that the patient is likely to have B-raf antagonist resistant cancer. In some embodiments, high c-met is low, moderate or high c-met expression determined, e.g., relative to c-met staining intensity of control cell pellets A549, H441, H1155, and HEK-293 as described herein. In some embodiments, high c-met is moderate or high c-met expression determined, e.g., relative to c-met staining intensity of control cell pellets A549, H441, H1155, and HEK-293 as described herein. In some embodiments, “low” c-met is low or no c-met expression determined, e.g., relative to c-met staining intensity of control cell pellets A549, H441, H1155, and HEK-293 as described herein. In some embodiments, “low” c-met expression is no c-met expression determined, e.g., relative to c-met staining intensity of control cell pellets A549, H441, H1155, and HEK-293 as described herein.

In one aspect, methods are provided for identifying a patient as at risk of developing resistance to a B-raf antagonist, comprising determining that the patient's cancer expresses c-met biomarker.

In one aspect, methods are provided of determining therapeutic efficacy of a B-raf antagonist for treating cancer in a patient comprising determining the presence of c-met biomarker and/or B-raf biomarker in a sample obtained from said patient by immunoassay, elisa, hybridization assay, PCR, 5′ nuclease assay, IHC, and/or RT-PCR, wherein the presence of c-met biomarker is indicative of B-raf antagonist being therapeutically effective to treat cancer in said subject. In some embodiments, the patient's cancer has been shown to express B-raf biomarker. In some embodiments, B-raf biomarker is mutant B-raf. In some embodiments, mutant B-raf is constitutively activated B-raf. In some embodiments, mutant B-raf is B-raf V600. In some embodiments, B-raf V600 is B-raf V600E. In some embodiments, mutant B-raf is one or more of B-raf V600K (GTG>AAG), V600R (GTG>AGG), V600E (GTG>GAA) and/or V600D (GTG>GAT). In some embodiments, mutant B-raf biomarker expression is determined using a method comprising (a) performing one or more of gene expression profiling, PCR (such as rtPCR or allele-specific PCR), RNA-seq, microarray analysis, SAGE, MassARRAY technique, or FISH on a sample (such as a patient cancer sample); and (b) determining expression of mutant B-raf biomarker in the sample. In some embodiments, mutant B-raf biomarker expression is determined using a method comprising (a) performing PCR on nucleic acid extracted from a patient cancer sample (such as a FFPE fixed patient cancer sample); and (b) determining expression of mutant B-raf biomarker in the sample. In some embodiments, the patient's cancer has been shown to express c-met biomarker. In some embodiments, c-met biomarker expression is determined using immunohistochemistry (IHC). In some embodiments, c-met expression is determined relative to c-met staining intensity of control cell pellets and high c-met expression is low, medium and strong c-met expression determined relative to cell line HEK-293, A549 and cell line H441. In some embodiments, c-met expression is determined relative to c-met staining intensity of control cell pellets and high c-met expression is medium and strong c-met expression determined relative to cell line A549 and cell line H441. In some embodiments, c-met expression is low c-met expression. In some embodiments, c-met expression is determined relative to c-met staining intensity of control cell pellets and low c-met expression is no or low c-met expression determined relative to cell line H1155 and cell line HEK-293. In some embodiments, c-met expression is determined relative to c-met staining intensity of control cell pellets and low c-met expression is no c-met expression determined relative to cell line H1155. In some embodiments, c-met biomarker expression is nucleic acid expression and is determined in a sample from the patient using PCR, RNA-seq, microarray analysis, SAGE, MassARRAY technique, or FISH. In some embodiments, c-met biomarker expression is determined using phospho-ELISA. In some embodiments, c-met biomarker expression is phospho-met expression. In some embodiments, c-met biomarker expression is determined by determining expression of hepatocyte growth factor (HGF) (e.g., using ELISA). In some embodiments, HGF expression is autocrine. In some embodiments, HGF is expressed in tumor or tumor stroma (e.g., determined using IHC. In some embodiments, expression is determined in the patient's serum (e.g., determined using ELISA). In some embodiments, cancer is melanoma, colorectal, breast, ovarian or thyroid. In some embodiments, cancer is melanoma. In some embodiments, the cancer is papillary thyroid.

In one aspect, the invention provides methods for determining prognosis for a melanoma patient, comprising determining expression of c-met biomarker in a sample from the patient, wherein c-met biomarker is HGF and expression of HGF is prognostic for cancer in the subject. In some embodiments, increased HGF expression is prognostic of, e.g., decreased progression-free survival and/or decreased overall survival when the patient is treated with B-raf inhibitor (e.g., vemurafenib). In some embodiments, HGF expression is determined in patient serum, e.g., using ELISA. In some embodiments, HGF expression in patient serum is above a median HGF expression level (such as a median HGF expression level in a population). In some embodiments, HGF expression in patient serum is above, for example, about 330 ng/ml. In some embodiments, HGF expression in patient serum is above about 300 ng/ml, 310 ng/ml, 320 ng/ml, 330 ng/ml, 340 ng/ml, 350 ng/ml, 360 ng/ml, 370 ng/ml, 380 ng/ml, 390 ng/ml, 400 ng/ml, 420 ng/ml, 440 ng/ml, 460 ng/ml, 480 ng/ml, 500 ng/ml, or greater. In some embodiments, the patient is selected for treatment with an effective amount of c-met antagonist and B-raf antagonist. In some embodiments, the patient is treated with an effective amount of a c-met antagonist and B-raf antagonist. In some embodiments, the melanoma expresses (has been shown to express) B-raf V600.

In some embodiments, the patient's cancer has been shown to express B-raf biomarker. B-raf biomarker may be mutant B-raf. Mutant B-raf is constitutively activated B-raf. In some embodiments, mutant B-raf is B-raf V600. B-raf V600 may be B-raf V600E. A non-limiting exemplary list of mutant B-raf is: B-raf V600K (GTG>AAG), V600R (GTG>AGG), V600E (GTG>GAA) and/or V600D (GTG>GAT). In some embodiments, mutant B-raf polypeptide is detected. In some embodiment, mutant B-raf nucleic acid is detected. “V600E” refers to a mutation in BRAF (T>A) at nucleotide position 1799 that results in substitution of a glutamine for a valine at amino acid position 600 of B-raf. “V600E” is also known as “V599E” (1796T>A) under a previous numbering system (Kumar et al., Clin. Cancer Res. 9:3362-3368, 2003).

In some embodiments, mutant B-raf biomarker expression is determined using a method comprising (a) performing one or more of gene expression profiling, PCR (such as rtPCR or allele-specific PCR), RNA-seq, 5′ nuclease assay (e.g., TaqMan), microarray analysis, SAGE, MassARRAY technique, or FISH on a sample (such as a patient cancer sample); and (b) determining expression of mutant B-raf biomarker in the sample. In some embodiments, mutant B-raf biomarker expression is determined using a method comprising (a) performing RT-PCR on nucleic acid extracted from a patient cancer sample (such as a FFPE fixed patient cancer sample); and (b) determining expression of mutant B-raf biomarker in the sample. In some embodiments, mutant B-raf biomarker expression is determined using a method comprising (a) performing PCR on nucleic acid extracted from a patient cancer sample (such as a FFPE fixed patient cancer sample); and (b) determining expression of mutant B-raf biomarker in the sample. In some embodiments, mutant B-raf biomarker expression is determined using a method comprising (a) hybridizing a first and second oligonucleotides to at least one variant of the B-raf target sequence; wherein said first oligonucleotide is at least partially complementary to one or more variants of the target sequence and said second oligonucleotide is at least partially complementary to one or more variants of the target sequence, and has at least one internal selective nucleotide complementary to only one variant of the target sequence; (b) extending the second oligonucleotide with a nucleic acid polymerase; wherein said polymerase is capable of extending said second oligonucleotide preferentially when said selective nucleotide forms a base pair with the target, and substantially less when said selective nucleotide does not form a base pair with the target; and (c) detecting the products of said oligonucleotide extension, wherein the extension signifies the presence of the variant of a target sequence to which the oligonucleotide has a complementary selective nucleotide. In some embodiments, the one or more variants of B-raf target sequence are wildtype B-raf and V600E B-raf.

In some embodiments, the patient's cancer has been shown to express c-met biomarker. C-met biomarker may be c-met polypeptide. In some embodiments, c-met biomarker expression is determined using immunohistochemistry (IHC). In some embodiments, high amount of c-met biomarker (e.g., as determined using c-met IHC or detection of HGF using, e.g., ELISA or IHC) indicates that the patient is likely to have B-raf antagonist resistant cancer. In some embodiments, high c-met is low, moderate or high c-met expression determined, e.g., relative to c-met staining intensity of control cell pellets A549, H441, H1155, and HEK-293 as described herein. In some embodiments, high c-met is moderate or high c-met expression determined, e.g., relative to c-met staining intensity of control cell pellets A549, H441, H1155, and HEK-293 as described herein. In some embodiments, “low” c-met is low or no c-met expression determined, e.g., relative to c-met staining intensity of control cell pellets A549, H441, H1155, and HEK-293 as described herein. In some embodiments, “low” c-met expression is no c-met expression determined, e.g., relative to c-met staining intensity of control cell pellets A549, H441, H1155, and HEK-293 as described herein. In some embodiments, the IHC score is 2. In some embodiments, the IHC score is 3. In some embodiments, the IHC score is 1. In some embodiments, the IHC score is 0. In some embodiments, high c-met biomarker expression is 50% or more of the tumor cells with moderate c-met staining intensity, combined moderate/high c-met staining intensity or high c-met staining intensity. In some embodiments, c-met biomarker expression is determined using phospho-ELISA. In some embodiments, c-met biomarker expression is phospho-met expression and, in some embodiments, is detected using an anti-phospho-c-met antibody.

C-met biomarker expression may be nucleic acid expression. In some embodiments, c-met biomarker is determined in a sample from the patient using PCR (such as rtPCR or allele-specific PCR), RNA-seq, microarray analysis, SAGE, MassARRAY technique, or FISH.

C-met biomarker may be determined by determining expression of hepatocyte growth factor (HGF). Thus, in some embodiment, c-met biomarker is HGF expression, and HGF expression is detected, e.g., in serum (e.g., using ELISA) or by IHC (e.g., or tumor or tumor stroma). HGF expression may be autocrine. HGF may be expressed in tumor stroma. In some embodiments, HGF expression is determined in the patient's serum. In some embodiments, HGF expression level is above median HGF expression level. In some embodiments, the median HGF expression level is about 330 pg/mL. In some embodiments, HGF expression in serum is greater than median HGF expression level. In some embodiments, HGF expression in serum is greater than about 330 pg/ml. In some embodiments, HGF expression in patient serum is above about 300 ng/ml, 310 ng/ml, 320 ng/ml, 330 ng/ml, 340 ng/ml, 350 ng/ml, 360 ng/ml, 370 ng/ml, 380 ng/ml, 390 ng/ml, 400 ng/ml, 420 ng/ml, 440 ng/ml, 460 ng/ml, 480 ng/ml, 500 ng/ml, or greater.

The c-met antagonist may be an antagonist anti-c-met antibody. In some embodiments, the anti-c-met antibody comprises a (a) HVR1-HC comprising sequence shown in SEQ ID NO: 1; (b) HVR2-HC comprising sequence shown in SEQ ID NO: 2; (c) HVR3-HC comprising sequence shown in SEQ ID NO: 3; (d) HVR1-LC comprising sequence shown in SEQ ID NO: 4; (e) HVR2-LC comprising sequence shown in SEQ ID NO: 5; and (f) HVR3-LC comprising sequence shown in SEQ ID NO: 6. In some embodiments, the anti-c-met antibody is monovalent and comprises (a) a first polypeptide comprising a heavy chain, said polypeptide comprising the sequence shown in SEQ ID NO: 11; (b) a second polypeptide comprising a light chain, the polypeptide comprising the sequence shown in SEQ ID NO: 12; and a third polypeptide comprising a Fc sequence, the polypeptide comprising the sequence shown in SEQ ID NO: 13, wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm.

In some embodiments, the c-met antagonist is one or more of crizotinib, tivantinib, carbozantinib, MGCD-265, ficlatuzumab, humanized TAK-701, rilotumumab, foretinib, h224G11, DN-30, MK-2461, E7050, MK-8033, PF-4217903, AMG208, JNJ-38877605, EMD1204831, INC-280, LY-2801653, SGX-126, RP1040, LY2801653, BAY-853474, GDC-0712, and/or LA480. In some embodiments, the c-met antagonist is crizotinib. In some embodiments, the c-met antagonist is tivantinib. In some embodiments, the c-met antagonist is GDC-0712.

In some embodiments, the B-raf antagonist is one or more of sorafenib, PLX4720, PLX-3603, GSK2118436, GDC-0879, N-(3-(5-(4-chlorophenyl)-1H-pyrrolo[2,3-b]pyridine-3-carbonyl)-2,4-difluorophenyl)propane-1-sulfonamide, vemurafenib, GSK 2118436, RAF265 (Novartis), XL281, ARQ736, BAY73-4506. In further embodiments, the B-raf antagonist is vemurafenib. In further embodiments, the B-raf antagonist is GSK 2118436. The B-raf antagonist may be selective for B-raf V600E.

The B-raf antagonist and the c-met antagonist may be administered simultaneously. The B-raf antagonist and the c-met antagonist may be administered sequentially. In some embodiments, the B-raf antagonist is administered prior to the c-met antagonist. In some embodiments, the c-met antagonist is administered prior to the B-raf antagonist.

In one aspect, provided are methods comprising administering at least one additional treatment (such as a cancer medicament) to said subject.

The cancer may be melanoma, colorectal, ovarian, breast or papillary thyroid. Other cancers are described herein. In some embodiments, the cancer is melanoma. In some embodiments, the cancer is resistant to B-raf antagonist. In some embodiments, the patient has been previously treated with B-raf antagonist. In some embodiments, the patient has not been previously treated with B-raf antagonist. In some embodiments, the patient is refractory to B-raf antagonist.

Moreover, the invention concerns methods for advertising a cancer medicament (e.g., a c-met antagonist) comprising promoting, to a target audience, the use of the cancer medicament for treating a patient with cancer based on expression of c-met biomarker, and in some embodiments, further based on expression of B-raf biomarker (e.g. mutant B-raf biomarker). Promotion may be conducted by any means available. In some embodiments, the promotion is by a package insert accompanying a commercial formulation of the c-met antagonist (such as an anti-c-met antibody). The promotion may also be by a package insert accompanying a commercial formulation of a second medicament (when treatment is combination therapy with a c-met antagonist and a second medicament, e.g., a B-raf antagonist such as vemurafenib). Promotion may be by written or oral communication to a physician or health care provider. In some embodiments, the promotion is by a package insert where the package insert provides instructions to receive therapy with c-met antagonist, and in some embodiments, in combination with a second medicament, such as a B-raf antagonist (such as vemurafenib). In some embodiments, the promotion is followed by the treatment of the patient with the c-met antagonist with or without the second medicament (e.g., vemurafenib). In some embodiments, the promotion is followed by the treatment of the patient with the second medicament with or without treatment with c-met antagonist. In some embodiments, the package insert indicates that the c-met antagonist is to be used to treat the patient if the patient's cancer sample expressed high c-met biomarker. In some embodiments, the package insert indicates that the c-met antagonist is not to be used to treat the patient if the patient's cancer sample expresses low c-met biomarker.

In some aspects, the invention features methods of instructing a patient with cancer (such as melanoma) expressing c-met biomarker by providing instructions to receive treatment with a c-met antagonist (for example, an anti-c-met antibody), and in some embodiments, treatment with a second medicament (such as B-raf antagonist, e.g. vemurafenib), for example, to increase survival of the patient, to decrease the patient's risk of cancer recurrence and/or to increase the patient's likelihood of survival. In some embodiments, the treatment comprises administering to the melanoma patient an anti-c-met antibody (e.g., MetMAb) administered in combination with a B-raf antagonist, such as vemurafenib. In some embodiments the method further comprises providing instructions to receive treatment with at least one chemotherapeutic agent. In certain embodiments the patient is treated as instructed by the method of instructing.

The invention also provides business methods, comprising marketing an c-met antagonist (e.g., anti-c-met antibody) for treatment of cancer (e.g., melanoma) in a human patient, wherein the patient's cancer expressed high (elevated) c-met biomarker expression, for example, to increase survival, decrease the patient's likelihood of cancer recurrence, and/or increase the patient's likelihood of survival. In some embodiments, the treatment comprises administering to a cancer patient an anti-c-met antibody (e.g., onartuzumab (MetMAb)), and in some embodiment, a second medicament (e.g., a B-raf antagonist, such as vemurafenib).

In one aspect, the invention provides diagnostic kits comprising one or more reagent for determining expression of a c-met biomarker in a sample from a cancer (e.g., melanoma) patient. The diagnostic kit is suitable for use with any of the methods described herein. In some embodiments, the kit further comprises instructions to use the kit to select a c-met medicament to treat the melanoma patient. In some embodiments, the treatment comprises administering to a cancer patient an anti-c-met antibody (e.g., onartuumab (MetMAb)), and in some embodiment, a second medicament (e.g., a B-raf antagonist, such as vemurafenib).

The invention also concerns articles of manufacture comprising, packaged together, a c-met antagonist in a pharmaceutically acceptable carrier and a package insert indicating that the c-met antagonist is for treating a patient with cancer based on expression of c-met biomarker. Treatment methods include any of the treatment methods disclosed herein.

### BRIEF DESCRIPTION OF THE SEVERAL VIEWS OF THE DRAWINGS

FIGS. 1A-1C: RTK ligands attenuated kinase inhibition in oncogene addicted cancer cell lines. FIG. 1A, Illustration depicting results from the RTK ligand matrix screen. Kinase addicted cancer cell lines were treated with an increasing concentration range of the appropriate kinase inhibitor in the presence or absence of RTK ligands (50 ng/mL). FIG. 1B, Summary of matrix screen results from forty-one kinase-addicted cancer cell lines co-treated with the effective kinase inhibitor and each of six individual RTK ligands. NR denotes no rescue, P denotes partial rescue and R denotes complete rescue. FIG. 1C, Cell viability assay demonstrating the diversity of RTK ligand effects on drug-treated cancer cell lines (72 h). Cells were co-treated with 50 ng/mL RTK ligand as indicated, with three different consequences observed—no rescue, partial rescue or complete rescue. Error bars represent mean+/−s.e.m.

FIGS. 2A-2C: pro-survival pathway re-activation correlated with RTK ligand rescue. FIG. 2A, Immunoblots showing the effect of acute RTK ligand treatment (50 ng/mL) on AKT and ERK phosphorylation following kinase inhibition (1 μM, 2 h). RTK ligand rescue is indicated, grey squares indicates complete rescue and black squares indicates partial rescue as determined by the initial screen (FIG. 1B). FIG. 2B, Cell viability assay demonstrated suppression of cell proliferation in three kinase addicted cancer cell lines following drug treatment (72 h). Cells were co-treated with 50 ng/mL RTK ligand in the presence of the appropriate secondary kinase inhibitor (0.5 μM) as indicated. PD: PD173074, Lap: lapatinib, Criz: crizotinib. Error bars represent mean+/−s.e.m. FIG. 2C, Immunoblots showing the effect of acute kinase inhibition (1 μM) in the presence and absence of RTK ligands (50 ng/mL, 2 h) on AKT and ERK phosphorylation. Cells were co-treated with secondary kinase inhibitor (0.5 μM) as appropriate. Sun: sunitinib, PD: PD173074, PLX: PLX4032, Lap: lapatinib, Erl: erlotinib, Criz: crizotinib.

FIGS. 3A-3G: HGF promoted lapatinib resistance in HER2 amplified cell lines. FIG. 3A, Immunoblots showing the suppression of apoptosis (cleaved PAPR) in AU565 HER2 amplified breast cancer cells following treatment with lapatinib (Lap, 1 μM), HGF (50 ng/mL) and crizotinib (Criz, 0.5 μM) as indicated. FIG. 3B, Immunoblots showing pMET and MET expression in a panel of HER2 amplified breast cancer cell lines. HGF rescue is indicated, black squares indicates partial rescue as determined by the initial screen (FIG. 1B). FIG. 3C, Syto 60 staining of AU565 HER2 amplified breast cancer cells treated with either lapatinib (Lap, 1 μM), HGF (50 ng/mL) or crizotinib (Criz, 0.5 μM) as indicated. Cells were treated every three days for the indicated times. Images are representative of 3 independent experiments and values indicate mean+/−s.d. FIG. 3D, Immunoblots showing the re-activation of pAKT and pERK in two MET positive (AU565, HCC1954) and one MET negative (BT474) HER2 amplified cell lines. Cells were treated with either lapatinib (Lap, 1 μM), HGF (50 ng/mL) or crizotinib (Criz, 0.5 μM) as indicated (2 h). FIG. 3E, Representative slides showing MET expression in HER2 positive (3+) breast cancer tissues. FIG. 3F, selection of higher MET expressing AU565 cells following 3× treatments with lapatinib (1 μM) and HGF (50 ng/mL). FIG. 3G. Syto 60 staining of HCC1954 HER2 amplified breast cancer cells treated with either lapatinib (5 μM) and crizotinib (1 μM) as indicated. Cells were treated twice weekly for the indicated times. Images are representative of 3 independent experiments and values indicate mean+/−s.d.

FIGS. 4A-4D: HGF promoted PLX4032 resistance in BRAF mutant melanoma cell lines. FIG. 4A, Left, immunoblots showing pMET and MET expression in a panel of BRAF mutant melanoma cell lines. HGF rescue is shown, grey squares denotes complete rescue, black squares denotes partial rescue and white squares denotes no rescue. Right, correlation between MET expression as determined by densitometry and the percent rescue in PLX4032 (1 μM) treated BRAF mutant melanoma lines in the presence of HGF (72 h). FIG. 4B, Immunoblots showing the re-activation of pERK in three MET positive (NAE, 624 MEL, A375) and two MET negative (M14, Hs693T) BRAF mutant cell lines. Cells were treated with PLX4032 (PLX, 1 μM), HGF (50 ng/mL) or crizotinib (Criz, 0.5 μM) as indicated (2 h). FIG. 4C, Syto 60 staining of 624MEL BRAF mutant melanoma cells treated with either PLX4032 (5 μM) and/or crizotinib (1 μM) as indicated. Cells were treated twice weekly for the indicated times. Images are representative of 3 independent experiments and values indicate mean+/−s.d. FIG. 4D, Tumor growth assay showing the effect of activating MET receptor using the 3D6 MET agonistic antibody on the effects of PLX4032 treatment in 928MEL xenografts. Mice, 10 per group, were treated with either Control antibody (anti-gp120), 3D6 (anti-MET agonistic antibody), RG7204 (PLX4032) or GDC-0712 (MET small molecular inhibitor) as indicated for 4 weeks. Error bars represent mean+/−s.e.m.

FIGS. 5A-5D: FIG. 5A, Immunoblots showing activation of PDGFR following stimulation with PDGF (50 ng/mL, 30 mins). FIG. 5B, Summary of screen results from six kinase addicted cancer cell lines co-treated with cisplatin and six individual RTK ligands. NR denotes no rescue. FIG. 5C, Cell viability assay demonstrating suppression of cell proliferation in three kinase addicted cancer cell lines following drug treatment (72 h). Cells were co-treated with 50 ng/mL RTK ligand in the presence of the appropriate secondary kinase inhibitor (0.5 μM) as indicated. PD: PD173074, Lap: lapatinib, Criz: crizotinib. Error bars represent mean+/−s.e.m. FIG. 5D, Immunoblots showing the effect of acute kinase inhibition (1 μM) in the presence or absence of RTK ligands (50 ng/mL, 2 h) on AKT and ERK phosphorylation. Cells were co-treated with secondary kinase inhibitor (0.5 μM) as appropriate. Criz: crizotinib, PD: PD173074, Lap: lapatinib.

FIGS. 6A & 6B: FIG. 6A, Immunoblots showing expression of MET, PDGFRα, IGF1Rβ, EGFR, HER2, HER3, FGFR1, FGFR2 and FGFR3 in the panel of 41 kinase addicted cancer cell lines from the matrix screen. RTK ligand rescue is indicated; grey squares denotes complete rescue, black squares denotes partial rescue, white squares denotes no rescue and hatched squares denotes ligand-associated kinase. X denotes removed sample, amp denotes amplified and mut denotes mutated. Equal loading was determined using β-tubulin. FIG. 6B, Table associating RTK expression with the ability of RTK ligands to rescue kinase-addicted cells from kinase inhibition. Statistical significance was determined using 2×2 contingency table. p values are given.

FIGS. 7A-7C: FIG. 7A, Immunoblots demonstrating activation of receptor without coupling to downstream survival signals in receptor expressing non-RTK ligand rescued cells. PLX: PLX4032, Lap: lapatinib. FIG. 7B, Immunoblots demonstrating activation of receptor with coupling to at least one downstream survival signal in receptor expressing non-RTK ligand rescued cells. PLX: PLX4032, TAE: TAE684, Erl: erlotinib. FIG. 7C, Immunoblots demonstrating the failure of RTK ligands to activate the appropriate receptor and corresponding downstream survival signals in receptor expressing non-RTK ligand rescued cells. PLX: PLX4032, TAE: TAE684, Erl: erlotinib.

FIGS. 8A-8D: FIG. 8A, Cell viability assay demonstrating suppression of cell proliferation in H3122 EML4-ALK translocated NSCLC cancer cell line following treatment with TAE684 or crizotinib treatment (72 h). Cells were co-treated with 50 ng/mL HGF. Error bars represent mean+/−s.e.m. FIG. 8B, Immunoblots showing the effect of acute TAE684 or crizotinib (1 μM) treatment in the presence and absence of HGF (50 ng/mL, 2 h) on AKT and ERK phosphorylation. FIG. 8C, Syto 60 staining of H2228 EML4-ALK translocated NSCLC cells treated with TAE684 (2 μM) in the presence and absence of HGF (50 ng/mL) as indicated. Cells were treated every 3 days for 9 days. FIG. 8D, Syto 60 staining of H358 EGF-like ligand-driven NSCLC cells treated with Erlotinib (5 μM) in the presence and absence of HGF (50 ng/mL) as indicated. Cells were treated every 3 days for 9 days. Images are representative of 3 independent experiments and values indicate mean+/−s.d.

FIGS. 9A & 9B: FIG. 9A, Cell viability assay demonstrating suppression of cell proliferation in two BRAF mutant cell lines following treatment with PLX4032 (72 h). Cells were co-treated with 50 ng/mL RTK ligand and crizotinib (Criz, 0.5 μM) as indicated. Error bars represent mean+/−s.e.m. FIG. 9B, Time course showing the sustained survival signals (pAKT and pERK) following HGF (50 ng/mL) stimulation in lapatinib (1 μM) treated AU565 HER2 amplified breast cancer cells.

FIG. 10: Rescue results of various RTK ligands in cells with BRAF V600F.

FIG. 11: Syto 60 cell staining of HCC1954 HER2 amplified breast cancer cells treated with either lapatinib (5 μM) and crizotinib (1 μM) as indicated. Cells were treated twice weekly for the indicated times. Images are representative of 3 independent experiments and values indicate mean+/−s.d.

FIGS. 12A & 12B: FIG. 12A, Immunoblots showing the re-activation of ERK in MET positive (NAE, 624MEL, 928MEL, A375) and MET negative (M14, Hs693T) BRAF mutant cell lines. Cells were treated with PLX4032 (PLX, 1 μM), HGF (50 ng/mL) or crizotinib (Criz, 0.5 μM) as indicated (2 h). FIG. 12B, Tumour growth assay showing the effect of activating MET using the 3D6 MET agonistic antibody on the growth inhibitory activity of PLX4032 in 928MEL and 624MEL xenografts. Mice (10 per group) were treated with either control antibody (anti-gp120), 3D6 (anti-MET agonistic antibody), RG7204 (PLX4032) or GDC-0712 (MET small molecular inhibitor) as indicated for 4 weeks, and tumour volumes were measured at the indicated times. Error bars represent mean+/−s.e.m. Differences between the 2 groups were determined using two-way ANOVA (*=0.0008).

FIG. 13: Progression-free survival and overall survival in metastatic melanoma patients treated with PLX4032. Patients were stratified into two groups based on their plasma HGF levels (green<median HGF; red>median HGF).

FIGS. 14A & 14B: FIG. 14A, Cell viability assay demonstrating the additive rescue from kinase inhibition by activating both the PI3K and MAPK pathways (72 h). AU565 sells were co-treated with lapatinib (1 μM) in combination with 10 ng/mL NRG1 or FGF. FIG. 14B, Cell viability assay demonstrating that inhibition of the PI3K pathway was more potent at reversing ligand—induced rescue than the MAPK pathway. Cells were treated with the appropriate kinase inhibitors in the presence of HGF (50 ng/mL). Cells were then treated with either 100 nM PI3K inhibitor (BEZ235) or MAPK inhibitor (AZD6244). Error bars represent mean+/−s.e.m.

FIG. 15: Immunoblots showing expression of MET, PDGFRα, IGF1Rβ, EGFR, HER2, HER3, FGFR1, FGFR2 and FGFR3 in the panel of 41 kinase addicted cancer cell lines from the matrix screen. RTK ligand rescue is indicated; grey squares denote complete rescue, dark grey squares denote partial rescue, white squares denote no rescue and black squares denote ligand-associated kinase. X denotes removed sample, amp denotes amplified and mut denotes mutated. Equal loading was determined using β-tubulin.

FIG. 16: Syto 60 staining of H2228 EML4-ALK translocated NSCLC cells treated with TAE684 (2 μM) in the presence or absence of HGF (50 ng/mL) as indicated. Cells were treated every 3 days for 9 days. Images are representative of 3 independent experiments and values indicate mean+/−s.d.

FIGS. 17A & 17B: FIG. 17A, Illustration depicting the analysis of 446 tested secreted factors on PLX4032 sensitivity in SK-MEL-28 cells. FIG. 17B, Summary of the results from the analysis of 446 tested secreted factors on SK-MEL-28 cells in the presence of 5 μM PLX4032 (72 h) in the presence of 50 ng/mL ligand. Graph represents the ligands form the original analysis and newly identified soluble factors that rescued SK-MEL-28 cells from PLX4032 sensitivity. Error bars represent mean+/−s.e.m.

FIG. 18: Syto 60 cell staining of A375 and 928MEL BRAF mutant melanoma cell lines treated with either PLX4032 (5 μM) and/or crizotinib (1 μM) as indicated. Cells were treated twice weekly for the indicated times. Images are representative of 3 independent experiments and values indicate mean+/−s.d.

FIGS. 19A & 19B: Tables summarizing results from the 928MEL and 624MEL xenograft studies.

FIG. 20 shows summary of ELISA results of HGF protein level in plasma from 126 metastatic melanoma patients pre-dose, cycle 1.

FIG. 21: IHC staining of MET in BRAF mutant melanoma cancer cells in culture.

FIG. 22: Cell viability assay demonstrating suppression of cell proliferation in HCC 1954 and AU565 following treatment with crizotinib (72 h syto 60 assay). Cells were co-treated with 50 ng/mL HGF in the presence of crizotinib (0.5 μM) (MET TKI) and lapatinib (EGFR/HER2 TKI).

FIG. 23: Immunoblots showing the effect of lapatinib (1 μM) in the presence of NRG1 (50 ng/mL, 2 h) on AKT and ERK phosphorylation. Cells were co-treated with erlotinib (0.5 μM) as indicated. Lap: lapatinib, Erl: erlotinib.

FIGS. 24A & 24B: FIG. 24A, Histogram (hatched) showing the frequency distribution of the log (HGF) levels, with empirical density (black) superimposed, from 126 metastatic melanoma patients enrolled on the BRIM2 trial, pre-dose cycle 1 (Kolmogorov-Smirnoff p-value for departures from normality is 0.18). FIG. 24B, Progression free survival (PFS) and overall survival (OS) in metastatic melanoma patients treated with PLX4032. Patients were stratified into three groups based on their plasma HGF level. Number of events/patients and medium time to event is shown for each group. The cox-proportional model of the outcome on the continuous outcome was used to calculate the hazard ratio and corresponding p-value.

FIGS. 25A-25E: FIG. 25A, Structure of GDC-0712. FIG. 25B, Enzyme IC50s for cMet and selected kinases. cMet potency was determined using phosphorylation of poly(Glu,Tyr) by activated cMet kinase domain, with detection by ELISA. Data is geometric mean of multiple determinations (n=5). Other kinase assays were carried out using Invitrogen SelectedScreen service according to Invitrogen standard protocols. All IC50s were determined with [ATP] at approximate values for Km. FIG. 25C, Potency and selectivity of GDC-0712 against selected RTKs in cell-based assays. All assays measured RTK autophosphorylation in the cell lines specified in the table, following 2-hour incubation with compound in the presence of 10% FBS. FIG. 25D, Kinase selectivity profiling data. GDC-0712 was assayed at 0.1 μM against a panel of 210 kinases using Invitrogen SelectScreen service. All kinases with >50% inhibition are listed. FIG. 25E, Graphic representation of GDC-0712 kinase selectivity. Percent inhibition of specific kinases at 0.1 μM compound is represented by size and color of circles overlaid on the human kinome.

FIG. 26: GDC-0712 was prepared according to the procedure outlined in the international patent application WO2007103308A2. Reagents and Conditions: (a) (EtO)₃CH, Meldrum's acid, 80° C., 76%; (b) Dowtherm, 220° C., 45%; (c) 3,4-difluoronitrobenzene, Cs₂CO₃, DMF, 100° C., 88%; (d) TFA, 70° C., 99%; (e) I₂, KOH, DMF, 50° C., 88%; (f) PMBCl, K₂CO₃, DMF, rt, 61%; (g) SnCl₂-dihydrate, EtOH, 65° C.; (h) CuI, indole-2-carboxylic acid, DMSO, K₂CO₃, 115° C., 56%; (i) EDCI, HOBt, ipr₂EtNH, DMF, 92%; (j) TFA, CH₂Cl₂, rt; (k) CH₃CHO, NaHB(OAc)₃, 77% over two steps; (1) TFA, 70° C., 73%.

### DETAILED DESCRIPTION OF EMBODIMENTS OF THE INVENTION

### I. Definitions

Herein, a “patient” is a human patient. The patient may be a “cancer patient”, i.e. one who is suffering or at risk for suffering from one or more symptoms of cancer. Moreover, the patient may be a previously treated cancer patient. The patient may be a “melanoma cancer patient”, i.e. one who is suffering or at risk for suffering from one or more symptoms of melanoma. Moreover, the patient may be a previously treated melanoma patient.

The term “c-met” or “Met”, as used herein, refers, unless indicated otherwise, to any native or variant (whether native or synthetic) c-met polypeptide. The term “wild type c-met” generally refers to a polypeptide comprising the amino acid sequence of a naturally occurring c-met protein.

The term “c-met variant” as used herein refers to a c-met polypeptide which includes one or more amino acid mutations in the native c-met sequence. Optionally, the one or more amino acid mutations include amino acid substitution(s).

An “anti-c-met antibody” is an antibody that binds to c-met with sufficient affinity and specificity. The antibody selected will normally have a sufficiently strong binding affinity for c-met, for example, the antibody may bind human c-met with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiments, the anti-c-met antibody can be used as a therapeutic agent in targeting and interfering with diseases or conditions wherein c-met activity is involved. Also, the antibody may be subjected to other biological activity assays, e.g., in order to evaluate its effectiveness as a therapeutic. Such assays are known in the art and depend on the target antigen and intended use for the antibody.

A “c-met antagonist” (interchangeably termed “c-met inhibitor”) is an agent that interferes with c-met activation or function. In a particular embodiment, a c-met inhibitor has a binding affinity (dissociation constant) to c-met of about 1,000 nM or less. In another embodiment, a c-met inhibitor has a binding affinity to c-met of about 100 nM or less. In another embodiment, a c-met inhibitor has a binding affinity to c-met of about 50 nM or less. In another embodiment, a c-met inhibitor has a binding affinity to c-met of about 10 nM or less. In another embodiment, a c-met inhibitor has a binding affinity to c-met of about 1 nM or less. In a particular embodiment, a c-met inhibitor is covalently bound to c-met. In a particular embodiment, a c-met inhibitor inhibits c-met signaling with an IC50 of 1,000 nM or less. In another embodiment, a c-met inhibitor inhibits c-met signaling with an IC50 of 500 nM or less. In another embodiment, a c-met inhibitor inhibits c-met signaling with an IC50 of 50 nM or less. In another embodiment, a c-met inhibitor inhibits c-met signaling with an IC50 of 10 nM or less. In another embodiment, a c-met inhibitor inhibits c-met signaling with an IC50 of 1 nM or less.

“C-met activation” refers to activation, or phosphorylation, of the c-met receptor. Generally, c-met activation results in signal transduction (e.g. that caused by an intracellular kinase domain of a c-met receptor phosphorylating tyrosine residues in c-met or a substrate polypeptide). C-met activation may be mediated by c-met ligand (HGF) binding to a c-met receptor of interest. HGF binding to c-met may activate a kinase domain of c-met and thereby result in phosphorylation of tyrosine residues in the c-met and/or phosphorylation of tyrosine residues in additional substrate polypeptides(s).

“B-raf activation” refers to activation, or phosphorylation, of the B-raf kinase. Generally, B-raf activation results in signal transduction.

The term “B-raf”, as used herein, refers, unless indicated otherwise, to any native or variant (whether native or synthetic) B-raf polypeptide. The term “wild type B-raf” generally refers to a polypeptide comprising the amino acid sequence of a naturally occurring B-raf protein.

The term “B-raf variant” as used herein refers to a B-raf polypeptide which includes one or more amino acid mutations in the native B-raf sequence. Optionally, the one or more amino acid mutations include amino acid substitution(s).

A “B-raf antagonist” (interchangeably termed “B-raf inhibitor”) is an agent that interferes with B-raf activation or function. In a particular embodiment, a B-raf inhibitor has a binding affinity (dissociation constant) to B-raf of about 1,000 nM or less. In another embodiment, a B-raf inhibitor has a binding affinity to B-raf of about 100 nM or less. In another embodiment, a B-raf inhibitor has a binding affinity to B-raf of about 50 nM or less. In another embodiment, a B-raf inhibitor has a binding affinity to B-raf of about 10 nM or less. In another embodiment, a B-raf inhibitor has a binding affinity to B-raf of about 1 nM or less. In a particular embodiment, a B-raf inhibitor inhibits B-raf signaling with an IC50 of 1,000 nM or less. In another embodiment, a B-raf inhibitor inhibits B-raf signaling with an IC50 of 500 nM or less. In another embodiment, a B-raf inhibitor inhibits B-raf signaling with an IC50 of 50 nM or less. In another embodiment, a B-raf inhibitor inhibits B-raf signaling with an IC50 of 10 nM or less. In another embodiment, a B-raf inhibitor inhibits B-raf signaling with an IC50 of 1 nM or less.

“V600E” refers to a mutation in the BRAF gene which results in substitution of a glutamine for a valine at amino acid position 600 of B-Raf. “V600E” is also known as “V599E” under a previous numbering system (Kumar et al., Clin. Cancer Res. 9:3362-3368, 2003).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

“Selective” or “greater affinity” means refers to an antagonist that binds more tightly (lower dissociation constant) to a mutant protein than to a wild-type protein. In some embodiments, greater affinity or selectivity is at least 2, 3, 4, 5, 6, 7, 8, 9, 10, 20, 50, 100, 200, 300, 400, 500 or more fold greater binding. As used herein, the term “B-raf-targeted drug” refers to a therapeutic agent that binds to B-raf and inhibits B-raf activation.

As used herein, the term “c-met-targeted drug” refers to a therapeutic agent that binds to c-met and inhibits c-met activation.

The term “constitutive” as used herein, as for example applied to receptor kinase activity, refers to continuous signaling activity of a receptor that is not dependent on the presence of a ligand or other activating molecules. Depending on the nature of the receptor, all of the activity may be constitutive or the activity of the receptor may be further activated by the binding of other molecules (e. g. ligands). Cellular events that lead to activation of receptors are well known among those of ordinary skill in the art. For example, activation may include oligomerization, e.g., dimerization, trimerization, etc., into higher order receptor complexes. Complexes may comprise a single species of protein, i.e., a homomeric complex. Alternatively, complexes may comprise at least two different protein species, i.e., a heteromeric complex. Complex formation may be caused by, for example, overexpression of normal or mutant forms of receptor on the surface of a cell. Complex formation may also be caused by a specific mutation or mutations in a receptor.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

A “tyrosine kinase inhibitor” is a molecule which inhibits to some extent tyrosine kinase activity of a tyrosine kinase such as a c-met receptor or B-raf.

A cancer or biological sample which “displays c-met and/or B-raf expression, amplification, or activation” is one which, in a diagnostic test, expresses (including overexpresses) c-met and/or B-raf, has amplified c-met and/or B-raf gene, and/or otherwise demonstrates activation or phosphorylation of a c-met and/or B-raf.

A cancer or biological sample which “does not display c-met and/or B-raf expression, amplification, or activation” is one which, in a diagnostic test, does not express (including overexpress) c-met and/or B-raf, does not have amplified c-met and/or B-raf gene, and/or otherwise does not demonstrate activation or phosphorylation of a c-met and/or B-raf.

A cancer or biological sample which “displays c-met and/or B-raf activation” is one which, in a diagnostic test, demonstrates activation or phosphorylation of C-met and/or B-raf. Such activation can be determined directly (e.g. by measuring C-met and/or B-raf phosphorylation by ELISA of IHC) or indirectly.

A cancer or biological sample which “does not display c-met and/or B-raf activation” is one which, in a diagnostic test, does not demonstrate activation or phosphorylation of a c-met and/or B-raf. Such activation can be determined directly (e.g. by measuring C-met and/or B-raf phosphorylation by ELISA or IHC) or indirectly.

A cancer or biological sample which “displays constitutive c-met and/or B-raf activation” is one which, in a diagnostic test, demonstrates constitutive activation or phosphorylation of a c-met and/or B-raf. Such activation can be determined directly (e.g. by measuring c-met and/or B-raf phosphorylation by ELISA) or indirectly.

A cancer or biological sample which “does not display c-met amplification” is one which, in a diagnostic test, does not have amplified c-met gene.

A cancer or biological sample which “displays c-met” is one which, in a diagnostic test, has amplified c-met gene.

A cancer or biological sample which “does not display constitutive c-met and/or B-raf activation” is one which, in a diagnostic test, does not demonstrate constitutive activation or phosphorylation of a c-met and/or B-raf. Such activation can be determined directly (e.g. by measuring c-met and/or B-raf phosphorylation by ELISA) or indirectly.

“Phosphorylation” refers to the addition of one or more phosphate group(s) to a protein, such as a B-raf and/or c-met, or substrate thereof.

A “phospho-ELISA assay” herein is an assay in which phosphorylation of one or more c-met and/or B-raf is evaluated in an enzyme-linked immunosorbent assay (ELISA) using a reagent, usually an antibody, to detect phosphorylated c-met and/or B-raf, substrate, or downstream signaling molecule. Preferably, an antibody which detects phosphorylated c-met and/or B-raf is used. The assay may be performed on cell lysates, preferably from fresh or frozen biological samples.

A cancer cell with “c-met overexpression or amplification” is one which has significantly higher levels of a c-met protein or gene compared to a noncancerous cell of the same tissue type. Such overexpression may be caused by gene amplification or by increased transcription or translation. C-met overexpression or amplification may be determined in a diagnostic or prognostic assay by evaluating increased levels of the c-met protein present on the surface of a cell (e.g. via an immunohistochemistry assay; IHC). Alternatively, or additionally, one may measure levels of C-met-encoding nucleic acid in the cell, e.g. via fluorescent in situ hybridization (FISH; see WO98/45479 published October, 1998), southern blotting, or polymerase chain reaction (PCR) techniques, such as quantitative real time PCR (qRT-PCR). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

A cancer cell which “does not overexpress or amplify c-met” is one which does not have higher than normal levels of c-met protein or gene compared to a noncancerous cell of the same tissue type.

The term “mutation”, as used herein, means a difference in the amino acid or nucleic acid sequence of a particular protein or nucleic acid (gene, RNA) relative to the wild-type protein or nucleic acid, respectively. A mutated protein or nucleic acid can be expressed from or found on one allele (heterozygous) or both alleles (homozygous) of a gene, and may be somatic or germ line. In the instant invention, mutations are generally somatic. Mutations include sequence rearrangements such as insertions, deletions, and point mutations (including single nucleotide/amino acid polymorphisms).

To “inhibit” is to decrease or reduce an activity, function, and/or amount as compared to a reference.

Protein “expression” refers to conversion of the information encoded in a gene into messenger RNA (mRNA) and then to the protein.

Herein, a sample or cell that “expresses” a protein of interest (such as a c-met receptor) is one in which mRNA encoding the protein, or the protein, including fragments thereof, is determined to be present in the sample or cell.

A “blocking” antibody or an antibody “antagonist” is one which inhibits or reduces biological activity of the antigen it binds. Preferred blocking antibodies or antagonist antibodies completely inhibit the biological activity of the antigen.

A “population” of patients refers to a group of patients with cancer, such as in a clinical trial, or as seen by oncologists following FDA approval for a particular indication, such as melanoma cancer therapy.

For the methods of the invention, the term “instructing” a patient means providing directions for applicable therapy, medication, treatment, treatment regimens, and the like, by any means, but preferably in writing, such as in the form of package inserts or other written promotional material.

For the methods of the invention, the term “promoting” means offering, advertising, selling, or describing a particular drug, combination of drugs, or treatment modality, by any means, including writing, such as in the form of package inserts. Promoting herein refers to promotion of therapeutic agent(s), such as an anti-c-met antibody and/or B-raf antagonist, for an indication, such as melanoma treatment, where such promoting is authorized by the Food and Drug Administration (FDA) as having been demonstrated to be associated with statistically significant therapeutic efficacy and acceptable safety in a population of subjects

The term “marketing” is used herein to describe the promotion, selling or distribution of a product (e.g., drug). Marketing specifically includes packaging, advertising, and any business activity with the purpose of commercializing a product.

For the purposes herein, a “previously treated” cancer patient has received prior cancer therapy.

“Refractory” cancer progresses even though an anti-tumor agent, such as a chemotherapeutic agent, is being administered to the cancer patient.

A “cancer medicament” is a drug effective for treating cancer. Examples of cancer medicaments include the chemotherapeutic agents and chemotherapy regimens noted below; c-met antagonists, including anti-c-met antibodies, such as MetMAb; B-raf antagonists.

The term “biomarker” or “marker” as used herein refers generally to a molecule, including a gene, mRNA, protein, carbohydrate structure, or glycolipid, the expression of which in or on a tissue or cell or secreted can be detected by known methods (or methods disclosed herein) and is predictive or can be used to predict (or aid prediction) for a cell, tissue, or patient's responsiveness to treatment regimes.

The “amount” or “level” of a biomarker associated with a decreased clinical benefit to a cancer (e.g. melanoma) patient refers to lack of detectable biomarker or a low detectable level in a biological sample, wherein the level of biomarker is associated with decreased clinical benefit to the patient. These can be measured by methods known to the expert skilled in the art and also disclosed by this invention. The expression level or amount of biomarker assessed can be used to determine the response to the treatment. In some embodiments, the amount or level of biomarker is determined using IHC (e.g., of patient tumor sample) and/or ELISA and/or 5′ nuclease assay and/or PCR (e.g., allele-specific PCR).

The terms “level of expression” or “expression level” in general are used interchangeably and generally refer to the amount of a polynucleotide, mRNA, or an amino acid product or protein in a biological sample. “Expression” generally refers to the process by which gene-encoded information is converted into the structures present and operating in the cell. Therefore, according to the invention “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. In some embodiments, “level of expression” refers to amount of a protein in a biological sample as determined using IHC.

By “patient sample” is meant a collection of similar cells obtained from a cancer patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may contain compounds which are not naturally intermixed with the tissue in nature such as preservatives, anticoagulants, buffers, fixatives, nutrients, antibiotics, or the like. Examples of tumor samples herein include, but are not limited to, tumor biopsies, circulating tumor cells, serum or plasma, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples. In one embodiment the sample comprises melanoma tumor sample.

An “effective response” of a patient or a patient's “responsiveness” to treatment with a medicament and similar wording refers to the clinical or therapeutic benefit imparted to a patient at risk for, or suffering from, cancer (e.g., melanoma) upon administration of the cancer medicament. Such benefit includes any one or more of: extending survival (including overall survival and progression free survival); resulting in an objective response (including a complete response or a partial response); or improving signs or symptoms of cancer, etc. In one embodiment, a biomarker is used to identify the patient who is expected to have greater progression free survival (PFS) when treated with a medicament (e.g., anti-c-met antibody), relative to a patient who does not express the biomarker at the same level.

“Survival” refers to the patient remaining alive, and includes overall survival as well as progression free survival.

“Overall survival” refers to the patient remaining alive for a defined period of time, such as 1 year, 5 years, etc from the time of diagnosis or treatment.

“Progression free survival” refers to the patient remaining alive, without the cancer progressing or getting worse.

By “extending survival” is meant increasing overall or progression free survival in a treated patient relative to an untreated patient (i.e. relative to a patient not treated with the medicament), or relative to a patient who does not express a biomarker at the designated level, and/or relative to a patient treated with an approved anti-tumor agent (such as chemotherapy regimen of erlotinib.

An “objective response” refers to a measurable response, including complete response (CR) or partial response (PR).

By “complete response” or “CR” is intended the disappearance of all signs of cancer in response to treatment. This does not always mean the cancer has been cured.

A “partial response” or “PR” refers to a decrease in the size of one or more tumors or lesions, or in the extent of cancer in the body, in response to treatment.

“Treatment” refers to both therapeutic treatment and prophylactic or preventative measures. Those in need of treatment include those already having a benign, pre-cancerous, or non-metastatic tumor as well as those in which the occurrence or recurrence of cancer is to be prevented.

The term “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, I, or II cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include melanoma, colorectal cancer, thyroid cancer (for example, papillary thyroid carcinoma), non-small cell lung cancer (NSCLC), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. In some embodiments, the cancer is melanoma; colorectal cancer; thyroid cancer, e.g., papillary thyroid cancer; or ovarian cancer.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin, and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovovin; novantrone; edatrexate; daunomycin; aminopterin; ibandronate; topoisomerase inhibitor RFS 2000; difluorometlhylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Herein, chemotherapeutic agents include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

Specific examples of chemotherapeutic agents or chemotherapy regimens herein include: alkylating agents (e.g. chlorambucil, bendamustine, or cyclophosphamide); nucleoside analogues or antimetabolites (e.g. fludarabine), fludarabine and cyclophosphamide (FC); prednisone or prednisolone; akylator-containing combination therapy, including cyclophosphamide, vincristine, prednisolone (CHOP), or cyclophosphamide, vincristine, prednisolone (CVP), etc.

A “target audience” is a group of people or an institution to whom or to which a particular medicament is being promoted or intended to be promoted, as by marketing or advertising, especially for particular uses, treatments, or indications, such as individual patients, patient populations, readers of newspapers, medical literature, and magazines, television or internet viewers, radio or internet listeners, physicians, drug companies, etc.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008)). Unless otherwise indicated, HVR residues and other residues in the variable domain (e.g., FR residues) are numbered herein according to Kabat et al., supra. In one embodiment, the c-met antibody herein comprises the HVRs of SEQ ID NOs: 1-6.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “immunoconjugate” is an antibody conjugated to one or more heterologous molecule(s), including but not limited to a cytotoxic agent.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “pharmaceutical formulation” refers to a sterile preparation that is in such form as to permit the biological activity of the medicament to be effective, and which contains no additional components that are unacceptably toxic to a subject to which the formulation would be administered.

A “sterile” formulation is aseptic or free from all living microorganisms and their spores.

A “kit” is any manufacture (e.g. a package or container) comprising at least one reagent, e.g., a medicament for treatment of cancer (e.g., melanoma, colorectal cancer), or a reagent (e.g., antibody) for specifically detecting a biomarker gene or protein. The manufacture is preferably promoted, distributed, or sold as a unit for performing the methods of the present invention.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### II. Cancer Medicaments

In one aspect, the present invention features the use of c-met antagonists and B-raf antagonists in combination therapy to treat a pathological condition, such as cancer, in a subject. In another aspect, the invention concerns selecting patients who can be treated with cancer medicaments based on expression of one or more of the biomarkers disclosed herein. Examples of cancer medicaments include, but are not limited to:
c-met antagonists, including anti-c-met antibodies.
B-raf antagonists.
Chemotherapeutic agents and chemotherapy regimens.
Other medicaments or combinations thereof in development, or approved, for treating cancer, e.g., melanoma.

Examples of c-met antagonists include, but are not limited to, soluble c-met receptors, soluble HGF variants, apatmers or peptibodies that bind c-met or HGF, c-met small molecules, anti-c-met antibodies and anti-HGF antibodies.

In one embodiment, the c-met antagonist is an antibody, e.g. directed against or which binds to c-met. The antibody herein includes: monoclonal antibodies, including a chimeric, humanized or human antibodies. In one embodiment, the antibody is an antibody fragment, e.g., a Fv, Fab, Fab′, one-armed antibody, scFv, diabody, or F(ab′)₂ fragment. In another embodiment, the antibody is a full length antibody, e.g., an intact IgG1 antibody or other antibody class or isotype as defined herein. In one embodiment, the antibody is monovalent. In another embodiment, the antibody is a one-armed antibody (i.e., the heavy chain variable domain and the light chain variable domain form a single antigen binding arm) comprising an Fc region, wherein the Fc region comprises a first and a second Fc polypeptide, wherein the first and second Fc polypeptides are present in a complex and form a Fc region that increases stability of said antibody fragment compared to a Fab molecule comprising said antigen binding arm. The one-armed antibody may be monovalent.

In another embodiment, the anti-c-met antibody is MetMAb (onartuzumab) or a biosimilar version thereof. MetMAb is disclosed in, for example, WO2006/015371; Jin et al, Cancer Res (2008) 68:4360. In another embodiment, the anti-c-met antibody comprises a heavy chain variable domain comprising one or more of (a) HVR1-HC comprising sequence GYTFTSYWLH (SEQ ID NO:1); (b) HVR2-HC comprising sequence GMIDPSNSDTRFNPNFKD (SEQ ID NO: 2); and/or (c) HVR3-HC comprising sequence ATYRSYVTPLDY (SEQ ID NO: 3). In some embodiments, the antibody comprises a light chain variable domain comprising one or more of (a) HVR1-LC comprising sequence KSSQSLLYTSSQKNYLA (SEQ ID NO: 4); HVR2-LC comprising sequence WASTRES (SEQ ID NO: 5); and/or (c) HVR3-LC comprising sequence QQYYAYPWT (SEQ ID NO: 6). In some embodiments the anti-c-met antibody comprises a heavy chain variable domain comprising (a) HVR1-HC comprising sequence GYTFTSYWLH (SEQ ID NO: 1); (b) HVR2-HC comprising sequence GMIDPSNSDTRFNPNFKD (SEQ ID NO: 2); and (c) HVR3-HC comprising sequence ATYRSYVTPLDY (SEQ ID NO: 3) and a light chain variable domain comprising (a) HVR1-LC comprising sequence KSSQSLLYTSSQKNYLA (SEQ ID NO: 4); HVR2-LC comprising sequence WASTRES (SEQ ID NO: 5); and (c) HVR3-LC comprising sequence QQYYAYPWT (SEQ ID NO: 6).

In any of the above embodiments, for example, an anti-c-met antibody can be humanized. In one embodiment, an anti-c-met antibody comprises HVRs as in any of the above embodiments, and further comprises an acceptor human framework, e.g. a human immunoglobulin framework or a human consensus framework.

In another aspect, an anti-c-met antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:7. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-c-met antibody comprising that sequence retains the ability to bind to human c-met. In certain embodiments, a total of 1 to 10 amino acids have been substituted, altered inserted and/or deleted in SEQ ID NO:7. In certain embodiments, substitutions, insertions, or deletions occur in regions outside the HVRs (i.e., in the FRs). Optionally, the anti-c-met antibody comprises the VH sequence in SEQ ID NO:7, including post-translational modifications of that sequence.

In another aspect, an anti-c-met antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:8. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-c-met antibody comprising that sequence retains the ability to bind to c-met. In certain embodiments, a total of 1 to 10 amino acids have been substituted, inserted and/or deleted in SEQ ID NO:8. In certain embodiments, the substitutions, insertions, or deletions occur in regions outside the HVRs (i.e., in the FRs). Optionally, the anti-c-met antibody comprises the VL sequence in SEQ ID NO: 8, including post-translational modifications of that sequence.

In yet another embodiment, the anti-c-met antibody comprises a VL region having at least 90%, 91%, 92%, 93%, 94%, 95%, 96%, 97%, 98%, 99%, or 100% sequence identity to the amino acid sequence of SEQ ID NO:8 and a VH region having at least 90%, 91%, 92%, 93%, 94%, 95%, 96%, 97%, 98%, 99%, or 100% sequence identity to the amino acid sequence of SEQ ID NO:7. In yet a further embodiment, the anti-c-met antibody comprises a HVR-L1 comprising amino acid sequence SEQ ID NO: 1; an HVR-L2 comprising amino acid sequence SEQ ID NO: 2; an HVR-L3 comprising amino acid sequence SEQ ID NO: 3; an HVR-H1 comprising amino acid sequence SEQ ID NO: 4; an HVR-H2 comprising amino acid sequence SEQ ID NO: 5; and an HVR-H3 comprising amino acid sequence SEQ ID NO: 6.

In another aspect, an anti-c-met antibody is provided, wherein the antibody comprises a VH as in any of the embodiments provided above, and a VL as in any of the embodiments provided above.

In a further aspect, the invention provides an antibody that binds to the same epitope as an anti-c-met antibody provided herein. For example, in certain embodiments, an antibody is provided that binds to the same epitope as or can by competitively inhibited by an anti-c-met antibody comprising a VH sequence of SEQ ID NO:7 and a VL sequence of SEQ ID NO:8.

In a further aspect of the invention, an anti-c-met antibody according to any of the above embodiment can be a monoclonal antibody, including a monovalent, chimeric, humanized or human antibody. In one embodiment, an anti-c-met antibody is an antibody fragment, e.g., a one-armed, Fv, Fab, Fab′, scFv, diabody, or F(ab′)₂ fragment. In another embodiment, the antibody is a full length antibody, e.g., an intact IgG1 or IgG4 antibody or other antibody class or isotype as defined herein. According to another embodiment, the antibody is a bispecific antibody. In one embodiment, the bispecific antibody comprises the HVRs or comprises the VH and VL regions described above.

In some embodiments, the anti-c-met antibody is monovalent, and comprises (or consisting of or consisting essentially of) (a) a first polypeptide comprising a heavy chain variable domain having the sequence: EVQLVESGGGLVQPGGSLRLSCAASGYTFTSYWLHWVRQAPGKGLEWVGMIDPSNSDTR FNPNFKDRFTISADTSKNTAYLQMNSLRAEDTAVYYCATYRSYVTPLDYWGQGTLVTVSS (SEQ ID NO:7), CH1 sequence, and a first Fc polypeptide; (b) a second polypeptide comprising a light chain variable domain having the sequence: DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO: 8), and CL1 sequence; and (c) a third polypeptide comprising a second Fc polypeptide, wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm. In some embodiments, the first polypeptide comprises Fc sequence CPPCPAPELLGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALPAPIEKTISKAKGQPREP QVYTLPPSREEMTKNQVSLSCAVKGFYPSDIAVEWESNGQPENNYKTTPPVLDSDGSFFLV SKLTVDKSRWQQGNVFSCSVMHEALHNHYTQKSLSLSPGK (SEQ ID NO: 9) and the second polypeptide comprises the Fc sequence CPPCPAPELLGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALPAPIEKTISKAKGQPREP QVYTLPPSREEMTKNQVSLWCLVKGFYPSDIAVEWESNGQPENNYKTTPPVLDSDGSFFL YSKLTVDKSRWQQGNVFSCSVMHEALHNHYTQKSLSLSPGK (SEQ ID NO: 10).

In another embodiments, the anti-c-met antibody is monovalent and comprises (a) a first polypeptide comprising a heavy chain, said polypeptide comprising the sequence: EVQLVESGGGLVQPGGSLRLSCAASGYTFTSYWLHWVRQAPGKGLEWVGMIDPSNSDTR FNPNFKDRFTISADTSKNTAYLQMNSLRAEDTAVYYCATYRSYVTPLDYWGQGTLVTVSS ASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPSV FLFPPKPKDTLMISRTPEVTCVVVDVSHEDPEVKFNWYVDGVEVHNAKTKPREEQYNSTY RVVSVLTVLHQDWLNGKEYKCKVSNKALPAPIEKTISKAKGQPREPQVYTLPPSREEMTK NQVSLSCAVKGFYPSDIAVEWESNGQPENNYKTTPPVLDSDGSFFLVSKLTVDKSRWQQG NVFSCSVMHEALHNHYTQKSLSLSPGK (SEQ ID NO: 11); (b) a second polypeptide comprising a light chain, the polypeptide comprising the sequence DIQMTQSPSSLSASVGDRVTITCKSSQSLLYTSSQKNYLAWYQQKPGKAPKLLIYWASTRE SGVPSRFSGSGSGTDFTLTISSLQPEDFATYYCQQYYAYPWTFGQGTKVEIKRTVAAPSVFI FPPSDEQLKSGTASVVCLLNNFYPREAKVQWKVDNALQSGNSQESVTEQDSKDSTYSLSST LTLSKADYEKHKVYACEVTHQGLSSPVTKSFNRGEC (SEQ ID NO: 12); and a third polypeptide comprising a Fc sequence, the polypeptide comprising the sequence DKTHTCPPCPAPELLGGPSVFLFPPKPKDTLMISRTPEVTCVVVDVSHEDPEVKFNWYVDG VEVHNAKTKPREEQYNSTYRVVSVLTVLHQDWLNGKEYKCKVSNKALPAPIEKTISKAKG QPREPQVYTLPPSREEMTKNQVSLWCLVKGFYPSDIAVEWESNGQPENNYKTTPPVLDSD GSFFLYSKLTVDKSRWQQGNVFSCSVMHEALHNHYTQKSLSLSPGK (SEQ ID NO: 13), wherein the heavy chain variable domain and the light chain variable domain are present as a complex and form a single antigen binding arm.

Other anti-c-met antibodies suitable for use in the methods of the invention are described herein and known in the art. For example, anti-c-met antibodies disclosed in WO05/016382 (including but not limited to antibodies 13.3.2, 9.1.2, 8.70.2, 8.90.3); an anti-c-met antibodies produced by the hybridoma cell line deposited with ICLC number PD 03001 at the CBA in Genoa, or that recognizes an epitope on the extracellular domain of the β chain of the HGF receptor, and said epitope is the same as that recognized by the monoclonal antibody); anti-c-met antibodies disclosed in WO2007/126799 (including but not limited to 04536, 05087, 05088, 05091, 05092, 04687, 05097, 05098, 05100, 05101, 04541, 05093, 05094, 04537, 05102, 05105, 04696, 04682); anti c-met antibodies disclosed in WO2009/007427 (including but not limited to an antibody deposited at CNCM, Institut Pasteur, Paris, France, on Mar. 14, 2007 under the number 1-3731, on Mar. 14, 2007 under the number 1-3732, on Jul. 6, 2007 under the number 1-3786, on Mar. 14, 2007 under the number 1-3724; an anti-c-met antibody disclosed in 20110129481; an anti-c-met antibody disclosed in US20110104176; an anti-c-met antibody disclosed in WO2009/134776; an anti-c-met antibody disclosed in WO2010/059654; an anti-c-met antibody disclosed in WO2011020925 (including but not limited to an antibody secreted from a hybridoma deposited at the CNCM, Institut Pasteur, Paris, France, on Mar. 12, 2008 under the number 1-3949 and the hybridoma deposited on Jan. 14, 2010 under the number 1-4273).

In one aspect, the anti-c-met antibody comprises at least one characteristic that promotes heterodimerization, while minimizing homodimerization, of the Fc sequences within the antibody fragment. Such characteristic(s) improves yield and/or purity and/or homogeneity of the immunoglobulin populations. In one embodiment, the antibody comprises Fc mutations constituting “knobs” and “holes” as described in WO2005/063816. For example, a hole mutation can be one or more of T366A, L368A and/or Y407V in an Fc polypeptide, and a cavity mutation can be T366W.

In some embodiments, the c-met antagonist is an anti-hepatocyte growth factor (HGF) antibody, for example, humanized anti-HGF antibody TAK701, rilotumumab, Ficlatuzumab, and/or humanized antibody 2B8 described in WO2007/143090. In some embodiments, the anti-HGF antibody is the anti-HGF antibody described in US7718174B2.

In some embodiments, the c-met antagonist is a c-met small molecule inhibitor. Small molecule inhibitors are preferably organic molecules other than binding polypeptides or antibodies as defined herein that bind, preferably specifically, to c-met. In some embodiments, the c-met small molecule inhibitor is a selective c-met small molecule inhibitor. In some embodiments, the c-met antagonist is a kinase inhibitor

C-met receptor molecules or fragments thereof that specifically bind to HGF can be used in the methods of the invention, e.g., to bind to and sequester the HGF protein, thereby preventing it from signaling. Preferably, the c-met receptor molecule, or HGF binding fragment thereof, is a soluble form. In some embodiments, a soluble form of the receptor exerts an inhibitory effect on the biological activity of the c-met protein by binding to HGF, thereby preventing it from binding to its natural receptors present on the surface of target cells. Also included are c-met receptor fusion proteins, examples of which are described below.

A soluble c-met receptor protein or chimeric c-met receptor proteins of the present invention includes c-met receptor proteins which are not fixed to the surface of cells via a transmembrane domain. As such, soluble forms of the c-met receptor, including chimeric receptor proteins, while capable of binding to and inactivating HGF, do not comprise a transmembrane domain and thus generally do not become associated with the cell membrane of cells in which the molecule is expressed. See, e.g., Kong-Beltran, M et al Cancer Cell (2004) 6(1): 75-84.

HGF molecules or fragments thereof that specifically bind to c-met and block or reduce activation of c-met, thereby preventing it from signaling, can be used in the methods of the invention.

Aptamers are nucleic acid molecules that form tertiary structures that specifically bind to a target molecule, such as a HGF or c-met polypeptide. The generation and therapeutic use of aptamers are well established in the art. See, e.g., U.S. Pat. No. 5,475,096. An HGF aptamer is a pegylated modified oligonucleotide, which adopts a three-dimensional conformation that enables it to bind to extracellular HGF. Additional information on aptamers can be found in U.S. Patent Application Publication No. 20060148748.

A peptibody is a peptide sequence linked to an amino acid sequence encoding a fragment or portion of an immunoglobulin molecule. Polypeptides may be derived from randomized sequences selected by any method for specific binding, including but not limited to, phage display technology. In a preferred embodiment, the selected polypeptide may be linked to an amino acid sequence encoding the Fc portion of an immunoglobulin. Peptibodies that specifically bind to and antagonize HGF or c-met are also useful in the methods of the invention.

In one embodiment, the c-met antagonist binds c-met extracellular domain. In some embodiments, the c-met antagonist binds c-met kinase domain. In some embodiments, the c-met antagonist competes for c-met binding with hepatocyte growth factor (HGF). In some embodiments, the c-met antagonist binds HGF.

In certain embodiments, the c-met antagonist is any one of: GDC-0712, SGX-523, Crizotinib (PF-02341066; 3-[(1R)-1-(2,6-dichloro-3-fluorophenyl)ethoxy]-5-(1-piperidin-4-ylpyrazol-4-yl)pyridin-2-amine; CAS no. 877399-52-5); JNJ-38877605 (CAS no. 943540-75-8), BMS-698769, PHA-665752 (Pfizer), SU5416, INC-280 (Incyte; SU11274 (Sugen; [(3Z)—N-(3-chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide; CAS no. 658084-23-2]), Foretinib (GSK1363089), XL880 (CAS no. 849217-64-7; XL880 is a inhibitor of met and VEGFR2 and KDR); MGCD-265 (MethylGene; MGCD-265 targets the c-MET, VEGFR1, VEGFR2, VEGFR3, Ron and Tie-2 receptors; CAS no. 875337-44-3), Tivantinib (ARQ 197; (−)-(3R,4R)-3-(5,6-dihydro-4H-pyrrolo[3,2,1-ij]quinolin-1-yl)-4-(1H-indol-3-yl)pyrrolidine-2,5-dione; see Munchi et al, Mol Cancer Ther June 2010 9; 1544; CAS no. 905854-02-6), LY-2801653 (Lilly), LY2875358 (Lilly), MP-470, Rilotumumab (AMG 102, anti-HGF monoclonal antibody), antibody 223C4 or humanized antibody 223C4 (WO2009/007427), humanized L2G7 (humanized TAK701; humanized anti-HGF monoclonal antibody); EMD 1214063 (Merck Sorono), EMD 1204831 (Merck Serono), NK4, Cabozantinib (XL-184, CAS no. 849217-68-1; carbozantinib is a dual inhibitor of met and VEGFR2), MP-470 (SuperGen; is a novel inhibitor of c-KIT, MET, PDGFR, Flt3, and AXL), Comp-1, Ficlatuzumab (AV-299; anti-HGF monoclonal antibody), E7050 (Cas no. 1196681-49-8; E7050 is a dual c-met and VEGFR2 inhibitor (Esai); MK-2461 (Merck; N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide; CAS no. 917879-39-1); MK8066 (Merck), PF4217903 (Pfizer), AMG208 (Amgen), SGX-126, RP1040, LY2801653, AMG458, EMD637830, BAY-853474, DP-3590. In certain embodiments, the c-met antagonist is any one or more of crizotinib, tivantinib, carbozantinib, MGCD-265, ficlatuzumab, humanized TAK-701, rilotumumab, foretinib, h224G11, DN-30, MK-2461, E7050, MK-8033, PF-4217903, AMG208, JNJ-38877605, EMD1204831, INC-280, LY-2801653, SGX-126, RP1040, LY2801653, BAY-853474, and/or LA480. In certain embodiments, the c-met antagonist is any one or more of crizotinib, tivantinib, carbozantinib, MGCD-265, ficlatuzumab, humanized TAK-701, rilotumumab, and/or foretinib. In some embodiments, the c-met antagonist is GDC-0712.

B-raf antagonists are known in the art and include, for example, sorafenib, PLX4720, PLX-3603, GSK2118436, GDC-0879, N-(3-(5-(4-chlorophenyl)-1H-pyrrolo[2,3-b]pyridine-3-carbonyl)-2,4-difluorophenyl)propane-1-sulfonamide, and those described in WO2007/002325, WO2007/002433, WO2009111278, WO2009111279, WO2009111277, WO2009111280 and U.S. Pat. No. 7,491,829. Other B-raf antagonists include, vemurafenib (also known as Zelobraf® and PLX-4032), GSK 2118436, RAF265 (Novartis), XL281, ARQ736, BAY73-4506. In some embodiments, the B-raf antagonist is a selective B-raf antagonist. In some embodiments, the B-raf antagonist is a selective antagonist of B-raf V600. In some embodiments, the B-raf antagonist is a selective antagonist of B-raf V600E. In some embodiments, B-raf V600 is B-raf V600E, B-raf V600K, and/or V600D. In some embodiments, B-raf V600 is B-raf V600R.

The B-raf antagonist may be a small molecule inhibitor. Small molecule inhibitors are preferably organic molecules other than polypeptides or antibodies as defined herein that bind, preferably specifically, to B-raf. In some embodiments, the B-raf antagonist is a kinase inhibitor. In some embodiments, the B-raf antagonist is an antibody, a peptide, a peptidomimetic, an aptomer or a polynubleotide.

In one embodiment, an antibody, e.g. the antibody used in the methods herein may incorporate any of the features, singly or in combination, as described in Sections 1-6 below:

I. Antibody Fragments

In certain embodiments, an antibody provided herein is an antibody fragment. Antibody fragments include, but are not limited to, Fab, Fab′, Fab′-SH, F(ab′)₂, Fv, and scFv fragments, a one-armed antibody, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

One-armed antibodies (i.e., the heavy chain variable domain and the light chain variable domain form a single antigen binding arm) are disclosed in, for example, WO2005/063816; Martens et al, Clin Cancer Res (2006), 12: 6144. For treatment of pathological conditions requiring an antagonistic function, and where bivalency of an antibody results in an undesirable agonistic effect, the monovalent trait of a one-armed antibody (i.e., an antibody comprising a single antigen binding arm) results in and/or ensures an antagonistic function upon binding of the antibody to a target molecule. Furthermore, the one-armed antibody comprising a Fc region is characterized by superior pharmacokinetic attributes (such as an enhanced half life and/or reduced clearance rate in vivo) compared to Fab forms having similar/substantially identical antigen binding characteristics, thus overcoming a major drawback in the use of conventional monovalent Fab antibodies. Techniques for making one-armed antibodies include, but are not limited to, “knob-in-hole” engineering (see, e.g., U.S. Pat. No. 5,731,168). MetMAb is an example of a one-armed antibody.

Antibody fragments can be made by various techniques, including but not limited to proteolytic digestion of an intact antibody as well as production by recombinant host cells (e.g. E. coli or phage), as described herein.

2. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

3. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

4. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

5. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for c-met and the other is for any other antigen (e.g. B-raf). In certain embodiments, bispecific antibodies may bind to two different epitopes of c-met. Bispecific antibodies may also be used to localize cytotoxic agents to cells which express c-met. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to c-met as well as another, different antigen, such as EGFR (see, US 2008/0069820, for example).

6. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest and the products screened for a desired activity, e.g., retained/improved antigen binding, decreased immunogenicity, or improved ADCC or CDC.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

In one embodiment, the medicament is an immunoconjugate comprising an antibody (such as a c-met antibody) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as his (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### Chemotherapeutic Agents

The combination therapy of the invention can additionally comprise treatment with one or more chemotherapeutic agent(s). The combined administration includes coadministration or concurrent administration, using separate formulations or a single pharmaceutical formulation, and consecutive administration in either order, wherein preferably there is a time period while both (or all) active agents simultaneously exert their biological activities. The chemotherapeutic agent, if administered, is usually administered at dosages known therefor, or optionally lowered due to combined action of the drugs or negative side effects attributable to administration of the chemotherapeutic agent. Preparation and dosing schedules for such chemotherapeutic agents may be used according to manufacturers' instructions or as determined empirically by the skilled practitioner.

Various chemotherapeutic agents that can be combined are disclosed above. In some embodiments, chemotherapeutic agents to be combined are selected from the group consisting of a taxoid (including docetaxel and paclitaxel), vinca (such as vinorelbine or vinblastine), platinum compound (such as carboplatin or cisplatin), aromatase inhibitor (such as letrozole, anastrazole, or exemestane), anti-estrogen (e.g. fulvestrant or tamoxifen), etoposide, thiotepa, cyclophosphamide, methotrexate, liposomal doxorubicin, pegylated liposomal doxorubicin, capecitabine, gemcitabine, COX-2 inhibitor (for instance, celecoxib), or proteosome inhibitor (e.g. PS342). In some embodiments, the chemotherapeutic agent is temozolomide and/or dacarbazine.

### III. Combination Therapies

In one aspect, provided are methods for treating a patient with cancer comprising administering an effective (e.g., a therapeutically effective) amount of B-raf antagonist and c-met antagonist. In some embodiments, the c-met antagonist is an anti-c-met antibody (e.g., MetMAb). In some embodiments, the treatment comprises administering an anti-c-met antibody (e.g., MetMAb) in combination with a B-raf antagonist, such as vemurafenib. In some embodiments, the anti-c-met antibody is MetMAb (onartuzumab).

In another aspect, provided are methods for treating a cancer patient who has increased likelihood of developing resistance to B-raf antagonist comprising administering an effective amount of B-raf antagonist and c-met antagonist.

In another aspect, provided are methods for increasing sensitivity to B-raf antagonist comprising administering to a cancer patient an effective amount of B-raf antagonist and c-met antagonist.

In another aspect, provided are methods for restoring sensitivity to B-raf antagonist comprising administering to a cancer patient an effective amount of B-raf antagonist and c-met antagonist.

In another aspect, provided are methods for extending period of B-raf antagonist sensitivity comprising administering to a cancer patient an effective amount of B-raf antagonist and c-met antagonist.

In another aspect, provided are methods for treating a patient with B-raf resistant cancer comprising administering an effective amount of B-raf antagonist and c-met antagonist.

In another aspect, provided are methods for extending response to B-raf antagonist comprising administering an effect amount of B-raf antagonist and c-met antagonist.

In another aspect, provided are methods of delaying or preventing development of HGF-mediated B-raf resistant cancer comprising administering an effective amount of B-raf antagonist and c-met antagonist.

In another aspect, methods are provided for treating a patient whose cancer has been shown to express B-raf biomarker (e.g., mutant B-raf biomarker) comprising determining whether the patient's cancer expresses c-met biomarker, and administering a B-raf antagonist and a c-met antagonist if the patient's cancer expresses c-met biomarker.

In another aspect, methods are provided for treating a patient whose cancer has been shown to express B-raf biomarker (e.g., mutant B-raf biomarker) comprising: (i) monitoring a patient being treated with a b-raf antagonist to determine if the patient's cancer develops expression of c-met biomarker, and (ii) modifying the treatment regimen of the patient to include a c-met antagonist in addition to the B-raf antagonist where the patient's cancer is shown to express c-met biomarker.

In another aspect, methods are provided for treating a patient whose cancer has been shown to express B-raf biomarker (e.g., mutant B-raf biomarker) comprising: (i) monitoring a patient being treated with B-raf antagonist to determine if the patient's cancer develops a resistance to the antagonist, (ii) testing the patient to determine whether the patient's cancer expresses c-met biomarker, and (iii) modifying the treatment regimen of the patient to include a c-met antagonist in addition to the B-raf antagonist where the patient's cancer is shown to express c-met biomarker.

The term cancer embraces a collection of proliferative disorders, including but not limited to pre-cancerous growths, benign tumors, and malignant tumors. Benign tumors remain localized at the site of origin and do not have the capacity to infiltrate, invade, or metastasize to distant sites. Malignant tumors will invade and damage other tissues around them. They can also gain the ability to break off from the original site and spread to other parts of the body (metastasize), usually through the bloodstream or through the lymphatic system where the lymph nodes are located. Primary tumors are classified by the type of tissue from which they arise; metastatic tumors are classified by the tissue type from which the cancer cells are derived. Over time, the cells of a malignant tumor become more abnormal and appear less like normal cells. This change in the appearance of cancer cells is called the tumor grade, and cancer cells are described as being well-differentiated (low grade), moderately-differentiated, poorly-differentiated, or undifferentiated (high grade). Well-differentiated cells are quite normal appearing and resemble the normal cells from which they originated. Undifferentiated cells are cells that have become so abnormal that it is no longer possible to determine the origin of the cells.

Cancer staging systems describe how far the cancer has spread anatomically and attempt to put patients with similar prognosis and treatment in the same staging group. Several tests may be performed to help stage cancer including biopsy and certain imaging tests such as a chest x-ray, mammogram, bone scan, CT scan, and MRI scan. Blood tests and a clinical evaluation are also used to evaluate a patient's overall health and detect whether the cancer has spread to certain organs.

To stage cancer, the American Joint Committee on Cancer first places the cancer, particularly solid tumors, in a letter category using the TNM classification system. Cancers are designated the letter T (tumor size), N (palpable nodes), and/or M (metastases). T1, T2, T3, and T4 describe the increasing size of the primary lesion; NO, N1, N2, N3 indicates progressively advancing node involvement; and MO and Ml reflect the absence or presence of distant metastases.

In the second staging method, also known as the Overall Stage Grouping or Roman Numeral Staging, cancers are divided into stages 0 to IV, incorporating the size of primary lesions as well as the presence of nodal spread and of distant metastases. In this system, cases are grouped into four stages denoted by Roman numerals I through IV, or are classified as “recurrent.” For some cancers, stage 0 is referred to as “in situ” or “Tis,” such as ductal carcinoma in situ or lobular carcinoma in situ for breast cancers. High grade adenomas can also be classified as stage 0. In general, stage I cancers are small localized cancers that are usually curable, while stage IV usually represents inoperable or metastatic cancer. Stage II and III cancers are usually locally advanced and/or exhibit involvement of local lymph nodes. In general, the higher stage numbers indicate more extensive disease, including greater tumor size and/or spread of the cancer to nearby lymph nodes and/or organs adjacent to the primary tumor. These stages are defined precisely, but the definition is different for each kind of cancer and is known to the skilled artisan.

Many cancer registries, such as the NCI's Surveillance, Epidemiology, and End Results Program (SEER), use summary staging. This system is used for all types of cancer. It groups cancer cases into five main categories:

In situ is early cancer that is present only in the layer of cells in which it began.

Localized is cancer that is limited to the organ in which it began, without evidence of spread.

Regional is cancer that has spread beyond the original (primary) site to nearby lymph nodes or organs and tissues.

Distant is cancer that has spread from the primary site to distant organs or distant lymph nodes.

Unknown is used to describe cases for which there is not enough information to indicate a stage.

In addition, it is common for cancer to return months or years after the primary tumor has been removed. Cancer that recurs after all visible tumor has been eradicated, is called recurrent disease. Disease that recurs in the area of the primary tumor is locally recurrent, and disease that recurs as metastases is referred to as a distant recurrence.

The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors. In some embodiments, the cancer is melanoma (e.g., B-raf mutant melanoma). In some embodiments, the cancer is colorectal cancer. In some embodiments, the cancer is breast cancer (e.g., Her2 positive breast cancer). In some embodiments, the cancer is papillary thyroid carcinoma. Other examples of cancers are provided in the Definitions.

In some embodiments, the patient's cancer has been shown to express B-raf biomarker. In some embodiments, B-raf biomarker is mutant B-raf. In some embodiments, mutant B-raf is B-raf V600. In some embodiments, B-raf V600 is B-raf V600E. In some embodiments, mutant B-raf is constitutively active.

In some embodiments, the patient's cancer has been shown to express c-met biomarker. Detection of c-met activity and expression is described herein.

In some embodiments, B-raf resistant cancer means that the cancer patient has progressed while receiving a B-raf antagonist therapy (i.e., the patient is “B-raf refractory”), or the patient has progressed within 1 month, 2 months, 3 months, 4 months, 5, months, 6 months, 7 months, 8 months, 9 months, 10 months, 11, months, 12 months, or more after completing a B-raf antagonist-based therapy regimen.

In some embodiments, vemurafenib resistant cancer is meant that the cancer patient has progressed while receiving vemurafenib-based therapy (i.e., the patient is “vemurafenib refractory”), or the patient has progressed within 1 month, 2 months, 3 months, 4 months, 5, months, 6 months, 7 months, 8 months, 9 months, 10 months, 11, months, 12 months, or more after completing a B-raf antagonist-based therapy regimen.

In some embodiments, resistance to, e.g., B-raf inhibitor develops (is acquired) after treatment with B-raf antagonist, or, e.g., following exposure to HGF (e.g., HGF-mediated resistance). In other embodiments, the patient (e.g., the patient having B-raf resistant cancer) has not been previously treated with a B-raf antagonist.

In some embodiments, the patient is currently being treated with B-raf antagonist. In some embodiments, the patient was previously treated with B-raf antagonist. In some embodiments, the patient was not previously treated with B-raf antagonist.

In one aspect, the cancer patient is treated with an additional cancer medicament. In some embodiments, the additional cancer medicament is a chemotherapeutic agent. In some embodiments, the additional cancer medicament is Yervoy. In some embodiments, the additional cancer medicament is a cancer immunotherapy agent. In some embodiments, the additional cancer medicament is a different (additional) B-raf antagonist. In some embodiments, the additional cancer medicament is a different (additional) c-met antagonist.

In one aspect, methods are provided for reducing B-raf phosphorylation in a cancer cell by comprising the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

In one aspect, methods are provided for reducing PI3K mediated signaling in a cancer cell comprising contacting the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

In one aspect, methods are provided for reducing PI3K mediated signaling in a cancer cell by comprising the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

In one aspect, methods are provided for reducing MAPk mediated signaling in a cancer cell by comprising the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

In one aspect, methods are provided for reducing AKT mediated signaling in a cancer cell by comprising the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

In one aspect, methods are provided for reducing ERK mediated signaling in a cancer cell by comprising the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

In one aspect, methods are provided for reducing B-raf-mediated signaling in a cancer cell by comprising the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

In one aspect, methods are provided for reducing growth and/or proliferation of a cancer cell, or increasing apoptosis of a cancer cell, comprising contacting the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

In one aspect, methods are provided for increasing apoptosis of a cancer cell comprising contacting the cell with a B-raf antagonist and a c-met antagonist. In some embodiments, the cell is resistant to B-raf antagonist (in some embodiments, has developed resistance to B-raf antagonist). In some embodiments, the cell expresses c-met biomarker.

The therapeutic agents used in the invention will be formulated, dosed, and administered in a fashion consistent with good medical practice. Factors for consideration in this context include the particular disorder being treated, the particular subject being treated, the clinical condition of the individual patient, the cause of the disorder, the site of delivery of the agent, the method of administration, the scheduling of administration, the drug-drug interaction of the agents to be combined, and other factors known to medical practitioners.

Therapeutic formulations are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

The therapeutic agents of the invention are administered to a human patient, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. An ex vivo strategy can also be used for therapeutic applications. Ex vivo strategies involve transfecting or transducing cells obtained from the subject with a polynucleotide encoding a c-met or B-raf antagonist. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

For example, if the c-met or B-raf antagonist is an antibody, the antibody is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Preferably the dosing is given by injections, most preferably intravenous or subcutaneous injections, depending in part on whether the administration is brief or chronic.

In another example, the c-met or B-raf antagonist compound is administered locally, e.g., by direct injections, when the disorder or location of the tumor permits, and the injections can be repeated periodically. The c-met or B-raf antagonist can also be delivered systemically to the subject or directly to the tumor cells, e.g., to a tumor or a tumor bed following surgical excision of the tumor, in order to prevent or reduce local recurrence or metastasis.

Administration of the therapeutic agents in combination typically is carried out over a defined time period (usually minutes, hours, days or weeks depending upon the combination selected). Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

The therapeutic agent can be administered by the same route or by different routes. For example, the B-raf and/or c-met antagonist in the combination may be administered by intravenous injection while the protein kinase inhibitor in the combination may be administered orally. Alternatively, for example, both of the therapeutic agents may be administered orally, or both therapeutic agents may be administered by intravenous injection, depending on the specific therapeutic agents. The sequence in which the therapeutic agents are administered also varies depending on the specific agents.

Depending on the type and severity of the disease, about 1 μg/kg to 100 mg/kg (e.g., 0.1-30 mg/kg) of each therapeutic agent is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until the cancer is treated, as measured by the methods described above. However, other dosage regimens may be useful. In one example, if the c-met or B-raf antagonist is an antibody, the antibody of the invention is administered every two to three weeks, at a dose ranging from about 5 mg/kg to about 150 mg/kg. If the c-met or B-raf antagonist is an oral small molecule compound, the drug may be administered daily at a dose ranging from about 25 mg/kg to about 50 mg/kg. Moreover, the oral compound of the invention can be administered either under a traditional high-dose intermittent regimen, or using lower and more frequent doses without scheduled breaks (referred to as “metronomic therapy”). When an intermittent regimen is used, for example, the drug can be given daily for two to three weeks followed by a one week break; or daily for four weeks followed by a two week break, depending on the daily dose and particular indication. The progress of the therapy of the invention is easily monitored by conventional techniques and assays.

The present application contemplates administration of the c-met and/or B-raf antagonist by gene therapy. See, for example, WO96/07321 published Mar. 14, 1996 concerning the use of gene therapy to generate intracellular antibodies.

### IV. Diagnostic Methods

In some embodiments, the patient herein is subjected to a diagnostic test e.g., prior to and/or during and/or after therapy.

In one aspect, provided are methods for determining c-met biomarker expression, comprising the step of determining whether a patient's cancer expresses c-met biomarker, wherein c-met biomarker expression indicates that the patient is likely to have B-raf antagonist resistant cancer. In some embodiments, the patient's cancer has been shown to express B-raf biomarker (such as mutant B-raf). In some embodiments, c-met biomarker expression is protein expression and is determined in a sample from the patient using IHC. In some embodiments, the patient is treated with B-raf antagonist and c-met antagonist.

In one aspect, provided are methods for determining c-met biomarker expression, comprising the step of determining whether a patient's cancer expresses c-met biomarker, wherein c-met biomarker expression indicates that the patient is likely to develop B-raf resistant cancer. In some embodiments, the patient's cancer has been shown to express B-raf biomarker (such as mutant B-raf). In some embodiments, c-met biomarker expression is protein expression and is determined in a sample from the patient using IHC. In some embodiments, the patient is treated with B-raf antagonist and c-met antagonist.

In one aspect, provided are methods for determining c-met biomarker expression, comprising the step of determining whether a patient's cancer expresses c-met biomarker, wherein c-met biomarker expression indicates that the patient is a candidate for treatment with c-met antagonist and B-raf antagonist: to increase sensitivity of the patient's cancer to B-raf antagonist, restore sensitivity of the patient's cancer to B-raf antagonist, to extend the period of sensitivity of the patient's cancer to B-raf antagonist, and/or to prevent development of HGF-mediated B-raf drug resistance in the patient's cancer. In some embodiments, the patient's cancer has been shown to express B-raf biomarker (such as mutant B-raf). In some embodiments, c-met biomarker expression is protein expression and is determined in a sample from the patient using IHC. In some embodiments, the patient is treated with B-raf antagonist and c-met antagonist.

The invention also relates to methods for selecting a therapy for a patient with cancer which has been shown to express B-raf biomarker (e.g., mutant B-raf biomarker) comprising determining expression of c-met biomarker in a sample from the patient, and selecting a cancer medicament based on the level of expression of the biomarker. In one embodiment, the patient is selected for treatment with a c-met antagonist (e.g., anti-c-met antibody) in combination with B-raf antagonist if the cancer sample expresses c-met biomarker. In some embodiments, the patient is treated for cancer using therapeutically effective amount of the c-met antagonist and B-raf antagonist. Thus, in some embodiments, the patient is selected for treatment with a c-met antagonist (e.g., anti-c-met antibody) if the patient's cancer sample expresses c-met biomarker, and (following the selection) the patient is treated for cancer using therapeutically effective amount of the c-met antagonist and B-raf antagonist. In another embodiment, the patient is selected for treatment with a cancer medicament other than c-met antagonist if the cancer sample expresses substantially undetectable levels of the c-met biomarker. In some embodiments, the patient is treated for cancer using therapeutically effective amounts of the cancer medicament other than c-met antagonist (e.g., treated with a B-raf antagonist). Thus, in some embodiments, the patient is selected for treatment with a cancer medicament (e.g., B-raf antagonist, e.g., vemurafenib) other than c-met antagonist if the cancer sample expresses c-met biomarker at a substantially undetectable level, and (following the selection) the patient is treated for cancer using therapeutically effective amount of the c-met antagonist.

In another aspect, the invention provides methods for identifying a patient as a candidate for treatment with a B-raf antagonist and a c-met antagonist, comprising determining that the patient's cancer expresses c-met biomarker. In some embodiments, the patient has been treated (previously treated) with B-raf antagonist. In some embodiments, the patient's cancer is resistant (e.g., has acquired resistance) to said B-raf antagonist.

In another aspect, the invention provides methods for identifying a patient as at risk of developing resistance to a B-raf antagonist, comprising determining that the patient's cancer expresses c-met biomarker. In some embodiments, the patient has been treated (previously treated) with B-raf antagonist. In some embodiments, the patient is being treated with B-raf antagonist.

In one aspect, the invention provides methods for determining prognosis for a melanoma patient, comprising determining expression of c-met biomarker in a sample from the patient, wherein c-met biomarker is HGF and expression of HGF is prognostic for cancer in the subject. In some embodiments, increased HGF expression is prognostic of, e.g., decreased progression-free survival and/or decreased overall survival when the patient is treated with B-raf inhibitor (e.g., vemurafenib). In some embodiments, HGF expression is determined in patient serum, e.g., using ELISA. In some embodiments, HGF expression in patient serum is above a median HGF expression level (such as a median HGF expression level in a population). In some embodiments, HGF expression in patient serum is above, for example, about 330 ng/ml. In some embodiments, HGF expression in patient serum is above about 300 ng/ml, 310 ng/ml, 320 ng/ml, 330 ng/ml, 340 ng/ml, 350 ng/ml, 360 ng/ml, 370 ng/ml, 380 ng/ml, 390 ng/ml, 400 ng/ml, 420 ng/ml, 440 ng/ml, 460 ng/ml, 480 ng/ml, 500 ng/ml, or greater. In some embodiments, the patient is selected for treatment with an effective amount of c-met antagonist and B-raf antagonist. In some embodiments, the patient is treated with an effective amount of a c-met antagonist and B-raf antagonist. HGF expression is detected, e.g., by IHC (e.g., or tumor or tumor stroma).

Methods for detection of c-met expression, activation and amplification are known in the art. In one aspect, c-met biomarker expression is determined using a method comprising: (a) performing IHC analysis of a sample (such as a patient cancer sample) with anti-c-met antibody; and b) determining expression of a c-met biomarker in the sample. In some embodiments, c-met IHC staining intensity is determined relative to a reference value. In some embodiments, high amount of c-met biomarker (e.g., as determined using c-met IHC or detection of HGF using, e.g., ELISA or IHC) indicates that the patient is likely to have B-raf antagonist resistant cancer. In some embodiments, high c-met is low, moderate or high c-met expression determined, e.g., relative to c-met staining intensity of control cell pellets A549, H441, H1155, and HEK-293 as described herein. In some embodiments, high c-met is moderate or high c-met expression determined, e.g., relative to c-met staining intensity of control cell pellets A549, H441, H1155, and HEK-293 as described herein. In some embodiments, “low” c-met is low or no c-met expression determined, e.g., relative to c-met staining intensity of control cell pellets A549, H441, H1155, and HEK-293 as described herein. In some embodiments, “low” c-met expression is no c-met expression determined, e.g., relative to c-met staining intensity of control cell pellets A549, H441, H1155, and HEK-293 as described herein. In some embodiments, c-met biomarker expression is determined using a c-met staining intensity scoring scheme is disclosed herein, e.g., in Table A. In some embodiments, the method further comprises stratifying the patients based on IHC score. In some embodiments, the IHC score is 1. In some embodiments, the IHC score is 0 and c-met expression is observed in the patient's cancer.

In some embodiments, c-met expression is polynucleotide expression. In some embodiments, the polynucleotide is RNA. In some embodiments, the polynucleotide is DNA. In some embodiments, the patient's cancer has been shown to express c-met copy number (e.g., by FISH analysis) greater than 2, greater than 3, greater than 4, greater than 5, greater than 6, greater than 7 greater than 8, or higher. In some embodiments, the c-met copy number is less than 8, less than 7, less than 6, less than 5, less than 4, less than 3.

It is contemplated that HGF may be detected according to the methods of the invention. Thus, in some embodiments, c-met biomarker is HGF, and in further embodiments, HGF expression is autocrine expression. In some embodiments, HGF expression is detected in the patient's cancer. In some embodiments, HGF expression is detected the patient's tumor stroma. In some embodiments, HGF expression is detected in patient serum, e.g., using ELISA.

In one aspect, c-met biomarker expression is determined using a method comprising the step of determining expression of c-met biomarker in the sample (such as a patient's cancer sample), wherein the patient's sample has been subjected to IHC analysis using an anti-c-met antibody. In some embodiments, c-met IHC staining intensity is determined relative to a reference value. In some embodiments, high amount of c-met biomarker (e.g., as determined using c-met IHC or detection of HGF using, e.g., ELISA or IHC) indicates that the patient is likely to have B-raf antagonist resistant cancer. In some embodiments, high c-met is low, moderate or high c-met expression determined, e.g., relative to c-met staining intensity of control cell pellets A549, H441, H1155, and HEK-293 as described herein. In some embodiments, high c-met is moderate or high c-met expression determined, e.g., relative to c-met staining intensity of control cell pellets A549, H441, H1155, and HEK-293 as described herein. In some embodiments, “low” c-met is low or no c-met expression determined, e.g., relative to c-met staining intensity of control cell pellets A549, H441, H1155, and HEK-293 as described herein. In some embodiments, “low” c-met expression is no c-met expression determined, e.g., relative to c-met staining intensity of control cell pellets A549, H441, H1155, and HEK-293 as described herein. In some embodiments, c-met biomarker expression is determined using a c-met staining intensity scoring scheme is disclosed herein, e.g., in Table A.

In some embodiments, IHC analysis further comprises morphological staining, either prior to or thereafter. In one embodiment, hematoxylin is use for staining cellular nucleic of the slides. Hematoxylin is widely available. An example of a suitable hematoxylin is Hematoxylin II (Ventana). When lighter blue nuclei are desired, a bluing reagent may be used following hematoxylin staining. Detection of c-met biomarker using IHC is disclosed herein, and a c-met staining intensity scoring scheme is disclosed herein, e.g., in Table A. As is noted herein, other biomarkers may be detected. Exemplary other biomarkers are disclosed herein. In some embodiments of any of the inventions disclosed herein, high c-met biomarker expression is met diagnostic positive clinical status as defined in accordance with Table A herein. In some embodiments of any of the inventions disclosed herein, low c-met biomarker expression is met diagnostic negative clinical status as defined in accordance with Table A herein.

In one aspect, c-met biomarker expression is determined using a method comprising: (a) performing one or more of western blotting, ELISA, phospho-ELISA, IHC using phospho-met antibody, IHC using anti-HGF antibody; and (b) determining expression of c-met biomarker (including, e.g., HGF) in the sample.

In one aspect, c-met activation is determined using a method comprising: (a) performing one or more of IHC using phospho-c-met antibody or phospho-ELISA; and (b) determining presence of phospho-c-met biomarker (e.g., phospho-c-met) in the sample.

In one aspect, c-met biomarker expression is determined using a method comprising the step of determining expression or activity of c-met downstream signaling pathway molecules, e.g., expression or activity of AKT (e.g., phospho-AKT), expression or activity of ERK (e.g., phospho-ERK).

In one aspect, c-met biomarker expression is determined using a method comprising: (a) performing gene expression profiling, PCR (such as rtPCR or allele-specific PCR), 5′ nuclease assay (e.g., Taq-man), RNA-seq, microarray analysis, SAGE, MassARRAY technique, in situ hybridization (e.g., for c-met and/or HGF mRNA), IHC (e.g., for c-met and/or HGF polypeptide) or FISH on a sample (such as a patient cancer sample); and b) determining expression of c-met biomarker in the sample.

As is noted herein, other biomarkers may be detected. Exemplary other biomarkers are disclosed herein. In some embodiments, ALK biomarker is detected. In some embodiments, one or more of FGF, FGFR, PDGF, and/or PGFR biomarker is detected.

Methods for detection of B-raf and mutant B-raf are known in the art and are commercially available. See, e.g., Hailat et al, Diagn Mol Pathol. 2012 March; 21(1):1-8. In some embodiments, V600E mutation (also known as V599E (1796T>A)) is detected using a method that comprises determining the presence of a single-base mutation (T>A) at nucleotide position 1799 in codon 600 of exon 15. This mutation can also result from the two-base mutation TG>AA at nucleotide positions 1799-1800. The two-base mutation can also be detected by evaluating position 1799. In some embodiments, a nucleic acid may also be evaluated for the presence of a substitution at position 1800. Other mutations also can occur at codon 600. These include V600K, V600D, and V600R. In some embodiments, a probe that detects a V600E mutation can also detect other codon 600 mutations, e.g., V600D, V600K and/or V600R. In some embodiments, a probe may also detect a mutation at codon 601.

The presence of a V600E mutation may be determined by assessing nucleic acid, e.g., genomic DNA or mRNA, for the presence of a base substitution at position 1799. In some embodiments, a nucleic acid analytical method is one or more of: hybridization using allele-specific oligonucleotides, primer extension, allele-specific ligation, sequencing, or electrophoretic separation techniques, e.g., singled-stranded conformational polymorphism (SSCP) and heteroduplex analysis. Exemplary assays include 5′ nuclease assays, allele-specific PCR, template-directed dye-terminator incorporation, molecular beacon allele-specific oligonucleotide assays, single-base extension assays, and mutations analysis using real-time pyrophosphate sequencing. Analysis of amplified sequences can be performed using various technologies such as microchips, fluorescence polarization assays, and matrix-assisted laser desorption ionization (MALDI) mass spectrometry. Two additional methods that can be used are assays based on invasive cleavage with Flap nucleases and methodologies employing padlock probes.

In some embodiments, mutant B-raf is B-raf V600E (B-raf polypeptide comprising a V600E mutation (GTG>GAG)). In some embodiments, mutant B-raf is one or more of B-raf V600K (GTG>AAG), V600R (GTG>AGG), V600E (GTG>GAA) and/or V600D (GTG>GAT). In some embodiments, mutant B-raf is mutant at residue V600. In some embodiments, a mutant B-raf polynucleotide comprises the T1799A mutation. In some embodiments, a mutant B-raf polynucleotide comprises a mutation in exon 11 and/or exon 15. In some embodiments, mutant B-raf expression is detected using a method comprising (a) performing one or more of gene expression profiling, PCR (such as rtPCR or allele-specific PCR), 5′ nuclease assay, IHC, hybridization assay, RNA-seq, microarray analysis, SAGE, MassARRAY technique, or FISH on a sample (such as a patient cancer sample); and (b) determining expression of mutant B-raf biomarker in the sample. In some embodiments, mutant B-raf biomarker expression is detected using a method comprising (a) performing PCR on nucleic acid extracted from a patient cancer sample (such as a FFPE fixed patient cancer sample); and (b) determining expression of mutant B-raf biomarker in the sample.

A sample from the patient is tested for expression of one or more of the biomarkers herein. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may contain compounds which are not naturally intermixed with the tissue in nature such as preservatives, anticoagulants, buffers, fixatives, nutrients, antibiotics, or the like. Examples of tumor samples herein include, but are not limited to, tumor biopsies, tumor cells, serum or plasma, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples. In one embodiment, the patient sample is a formalin-fixed paraffin-embedded (FFPE) tumor sample (e.g., a melanoma tumor sample or a colorectal cancer tumor sample or a sample of tumor stroma). The sample may be obtained prior to the patient's treatment with a cancer medicament (such as an anti-c-met antagonist). The sample may be obtained from the primary tumor or from a metastatic tumor. The sample may be obtained when the cancer is first diagnosed or, for example, after the tumor has metastasized. In some embodiments, the tumor sample is of lung, skin, lymph node, bone, liver, colon, thyroid, and/or ovary.

Various methods for determining expression of mRNA, protein, or gene amplification include, but are not limited to, gene expression profiling, polymerase chain reaction (PCR) including quantitative real time PCR (qRT-PCR), allele-specific PCR, RNA-Seq, FISH, microarray analysis, serial analysis of gene expression (SAGE), MassARRAY, proteomics, immunohistochemistry (IHC), etc. In some embodiments, protein expression is quantified. Such protein analysis may be performed using IHC, e.g., on patient tumor samples.

Various exemplary methods for determining biomarker expression will now be described in more detail.

I. Gene Expression Profiling

In general, methods of gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker &Barnes, Methods in Molecular Biology 106:247-283 (1999)); RNAse protection assays (Hod, Biotechniques 13:852-854 (1992)); and polymerase chain reaction (PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

2. Polymerase Chain Reaction (PCR) and 5′ Nuclease Assays

A sensitive and flexible quantitative method is PCR, which can be, for example, used to compare mRNA levels in different sample populations, in normal and tumor tissues, with or without drug treatment, to characterize patterns of gene expression, to discriminate between closely related mRNAs, and to analyze RNA structure. It is noted, however, that other nucleic acid amplification protocols (i.e., other than PCR) may also be used in the nucleic acid analytical methods described herein. For example, suitable amplification methods include ligase chain reaction (see, e.g., Wu & Wallace, Genomics 4:560-569, 1988); strand displacement assay (see, e.g., Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86:1173-1177, 1989); and self-sustained sequence replication (3SR) (Guatelli et al., Proc. Natl. Acad. Sci. USA 87:1874-1878, 1990; WO 92/08800). Alternatively, methods that amplify the probe to detectable levels can be used, such as Qβ-replicase amplification (Kramer & Lizardi, Nature 339:401-402, 1989; Lomeli et al., Clin. Chem. 35:1826-1831, 1989). A review of known amplification methods is provided, for example, by Abramson and Myers in Current Opinion in Biotechnology 4:41-47, 1993.

mRNA may be isolated from the starting tissue sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, in some embodiments, the first step in gene expression profiling by PCR is the reverse transcription of the RNA template into cDNA, followed by its exponential amplification in a PCR reaction. In other embodiments, a combined reverse-transcription-polymerase chain reaction (RT-PCR) reaction may be used, e.g., as described in U.S. Pat. Nos. 5,310,652; 5,322,770; 5,561,058; 5,641,864; and 5,693,517. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif., USA), following the manufacturer's instructions. The derived cDNA can then be used as a template in the subsequent PCR reaction.

TaqMan® or “5′-nuclease assay”, as described in U.S. Pat. Nos. 5,210,015; 5,487,972; and 5,804,375; and Holland et al., 1988, Proc. Natl. Acad. Sci. USA 88:7276-7280, may be used. TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data. The hybridization probe employed in the assay can be an allele-specific probe that, e.g., discriminates between the mutant and wildtype alleles of BRAF at the V600E mutation site. Alternatively, the method can be performed using an allele-specific primer and a labeled probe that binds to amplified product.

Any method suitable for detecting degradation product can be used in a 5′ nuclease assay. Often, the detection probe is labeled with two fluorescent dyes, one of which is capable of quenching the fluorescence of the other dye. The dyes are attached to the probe, preferably one attached to the 5′ terminus and the other is attached to an internal site, such that quenching occurs when the probe is in an unhybridized state and such that cleavage of the probe by the 5′ to 3′ exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated herein by reference, describe alternative methods for detecting the degradation of probe which occurs concomitant with amplification. 5′-Nuclease assay data may be initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and P-actin.

In some embodiments, the probe that detects V600E, e.g., TTS155-BRAF_MU, also detects V600D (1799_{—}1800TG>AT) and V600K (1798_{—}1799GT>AA). In some embodiments, a probe that detects a V600E mutation also detects K601E (1801A>G) and V600R (1798_{—}1799GT>AG).

In some embodiments, a sequence substantially identical to a probe sequence can be used. Sequences that are substantially identical to the probe sequences include those that hybridize to the same complementary sequence as the probe. Thus, in some embodiments, probe sequences for use in the invention comprise at least 15 contiguous nucleotides, sometimes at least 16, 17, 18, 19, 20, 21, 22, 23, 24, 25, 26, 27, 28, 29, or 30 contiguous nucleotides of the. In some embodiments, a primer has at least 27, 28, 29, or 30 contiguous nucleotide of TTS155-BRAF_MU or TTS148-BRAF_WT. In other embodiments, primers for use in the invention have at least 80% identity, in some embodiments at least 85% identity, and in other embodiments at least 90% or greater identity to TTS155-BRAF_MU or TTS148-BRAF_WT.

The steps of a representative protocol for profiling gene expression using fixed, paraffin-embedded tissues as the RNA source, including mRNA isolation, purification, primer extension and amplification are given in various published journal articles (for example: Hailat et al, Diagn Mol Pathol. 2012 March; 21(1):1-8; Godfrey et al., J. Molec. Diagnostics 2: 84-91 (2000); Specht et al., Am. J. Pathol. 158: 419-29 (2001)). Briefly, in some embodiments, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W., Genome Res. 12(4):656-64 (2002), or by the BLAST software including its variations. Subsequent steps follow well established methods of PCR primer and probe design.

In order to avoid non-specific signals, it can be important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Rozen and Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

Factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

In another aspect, allele-specific amplification of a target nucleic acid may be used to detect the presence or absence of a nucleic acid mutation. The amplification involves the use of an allele-specific primer.

In one embodiment, the present invention is a method of allele-specific amplification of a variant of a target sequence, which exists in the form of several variant sequences, the method comprising: providing a sample, possibly containing at least one variant of a target sequence; providing a first oligonucleotide, at least partially complementary to one or more variants of the target sequence; providing a second oligonucleotide, at least partially complementary to one or more variants of the target sequence, but having at least one internal selective nucleotide complementary to only one variant of the target sequence; providing conditions suitable for the hybridization of said first and second oligonucleotides to at least one variant of the target sequence; providing conditions suitable for the oligonucleotide extension by a nucleic acid polymerase; wherein said polymerase is capable of extending said second oligonucleotide when it is hybridized to the variant of the target sequence for which it has said complementary internal selective nucleotide, and substantially less when said second oligonucleotide is hybridized to the variant of the target sequence for which it has a non-complementary internal selective nucleotide; and repeating the sequence of hybridization and extension steps multiple times.

In some embodiments of the invention, the amplification involves the polymerase chain reaction, i.e. repeated cycles of template denaturation, annealing (hybridization) of the oligonucleotide primer to the template, and extension of the primer by the nucleic acid polymerase. In some embodiments, annealing and extension occur at the same temperature step.

In some embodiments, the amplification reaction involves a hot start protocol. In the context of allele-specific amplification, the selectivity of the allele-specific primers with respect to the mismatched target may be enhanced by the use of a hot start protocol. Many hot start protocols are known in the art, for example, the use of wax, separating the critical reagents from the rest of the reaction mixture (U.S. Pat. No. 5,411,876), the use of a nucleic acid polymerase, reversibly inactivated by an antibody (U.S. Pat. No. 5,338,671), a nucleic acid polymerase reversibly inactivated by an oligonucleotide that is designed to specifically bind its active site (U.S. Pat. No. 5,840,867) or the use of a nucleic acid polymerase with reversible chemical modifications, as described e.g. in U.S. Pat. Nos. 5,677,152 and 5,773,528.

In some embodiments of the invention, the allele-specific amplification assay is real-time PCR assay. In a real-time PCR assay, the measure of amplification is the “threshold cycle” or Ct value. In the context of the allele-specific real-time PCR assay, the difference in Ct values between the matched and the mismatched templates is a measure of discrimination between the alleles or the selectivity of the assay. A greater difference indicates a greater delay in amplification of the mismatched template and thus a greater discrimination between alleles. Often the mismatched template is present in much greater amounts than the matched template. For example, in tissue samples, only a small fraction of cells may be malignant and carry the mutation targeted by the allele-specific amplification assay (“matched template”). The mismatched template present in normal cells may be amplified less efficiently, but the overwhelming numbers of normal cells will overcome any delay in amplification and erase any advantage of the mutant template. To detect rare mutations in the presence of the wild-type template, the specificity of the allele-specific amplification assay is critical. The COBAS® 4800 BRAF V600 Mutation Test is commercially available and utilizes real-time PCR technology. Each target-specific, oligonucleotide probe in the reaction is labeled with a fluorescent dye that serves as a reporter, and with a quencher molecule that absorbs (quenches) fluorescent emissions from the reporter dye within an intact probe. During each cycle of amplification, probe complementary to the single-stranded DNA sequence in the amplicon binds and is subsequently cleaved by the 5′ to 3′ nuclease activity of the Z05 DNA polymerase. Once the reporter dye is separated from the quencher by this nuclease activity, fluorescence of a characteristic wavelength can be measured when the reporter dye is excited by the appropriate spectrum of light. Two different reporter dyes are used to label the target-specific BRAF wild-type (WT) probe and the BRAF V600E mutation (MUT) probe. Amplification of the two BRAF sequences can be detected independently in a single reaction well by measuring fluorescence at the two characteristic wavelengths in dedicated optical channels.

In one embodiment, primers that differentiate between B-raf and V600E B-raf are utilized, according to US Patent Publication No. 2011/0311968.

In some embodiments, mutant B-raf polynucleotide (e.g., DNA) is detected using a method comprising (a) performing PCR on nucleic acid (e.g., genomic DNA) extracted from a patient cancer sample (such as a FFPE fixed patient cancer sample); and (b) determining expression of mutant B-raf polynucleotide in the sample. In some embodiments, mutant B-raf polynucleotide expression is detected using a method comprising (a) hybridizing a first and second oligonucleotides to at least one variant of the B-raf target sequence; wherein said first oligonucleotide is at least partially complementary to one or more variants of the target sequence and said second oligonucleotide is at least partially complementary to one or more variants of the target sequence, and has at least one internal selective nucleotide complementary to only one variant of the target sequence; (b) extending the second oligonucleotide with a nucleic acid polymerase; wherein said polymerase is capable of extending said second oligonucleotide preferentially when said selective nucleotide forms a base pair with the target, and substantially less when said selective nucleotide does not form a base pair with the target; and (c) detecting the products of said oligonucleotide extension, wherein the extension signifies the presence of the variant of a target sequence to which the oligonucleotide has a complementary selective nucleotide. In some embodiments, mutant B-raf polynucleotide (e.g., DNA) is detected using a method comprising (a) performing PCR on nucleic acid (e.g., genomic DNA) extracted from a patient cancer sample (such as a FFPE fixed patient cancer sample); and (b) determining expression of mutant B-raf polynucleotide in the sample. In some embodiments, mutant B-raf polynucleotide (e.g., DNA) is detected using a method comprising (a) isolating DNA (e.g., gemonic DNA) from a patient cancer sample (such as a FFPE fixed patient cancer sample); (b) performing PCR on the DNA extracted from a patient cancer sample; and (c) determining expression of mutant B-raf polynucleotide in the sample.

In some embodiments, mutant B-raf polynucleotide expression is detected using a method comprising (a) isolating DNA (e.g., gemonic DNA) from a patient cancer sample (such as a FFPE fixed patient cancer sample); (b) hybridizing a first and second oligonucleotides to at least one variant of the B-raf target sequence in the DNA; wherein said first oligonucleotide is at least partially complementary to one or more variants of the target sequence and said second oligonucleotide is at least partially complementary to one or more variants of the target sequence, and has at least one internal selective nucleotide complementary to only one variant of the target sequence; (c) extending the second oligonucleotide with a nucleic acid polymerase; wherein said polymerase is capable of extending said second oligonucleotide preferentially when said selective nucleotide forms a base pair with the target, and substantially less when said selective nucleotide does not form a base pair with the target; and (d) detecting the products of said oligonucleotide extension, wherein the extension signifies the presence of the variant of a target sequence to which the oligonucleotide has a complementary selective nucleotide. In some embodiments, mutant B-raf polynucleotide expression is detected using a method comprising (a) hybridizing a first and second oligonucleotides to at least one variant of the B-raf target sequence; wherein said first oligonucleotide is at least partially complementary to one or more variants of the target sequence and said second oligonucleotide is at least partially complementary to one or more variants of the target sequence, and has at least one internal selective nucleotide complementary to only one variant of the target sequence; (b) extending the second oligonucleotide with a nucleic acid polymerase; wherein said polymerase is capable of extending said second oligonucleotide preferentially when said selective nucleotide forms a base pair with the target, and substantially less when said selective nucleotide does not form a base pair with the target; and (c) detecting the products of said oligonucleotide extension, wherein the extension signifies the presence of the variant of a target sequence to which the oligonucleotide has a complementary selective nucleotide.

In some embodiments, mutant B-raf polynucleotide (e.g., DNA) is detected using a method comprising (a) performing PCR on nucleic acid (e.g., genomic DNA) extracted from a patient cancer sample (such as a FFPE fixed patient cancer sample); (b) determining expression of mutant B-raf polynucleotide by sequencing the PCR amplified nucleic acid. In some embodiments, mutant B-raf polynucleotide (e.g., DNA) is detected using sequencing (e.g., Sanger sequence or pyrosequencing).

3. Other Nucleic Acid Mutation Detection Methods

The presence (or absence) of a nucleic acid mutation (e.g., (GTG>GAA) at nucleotide position 1799 that results in substitution of a glutamine for a valine at amino acid position 600 of B-raf) can also be detected by direct sequencing. Methods include dideoxy sequencing-based methods and methods such as Pyrosequencing™ of oligonucleotide-length products. Such methods often employ amplification techniques such as PCR. Another similar method for sequencing does not require use of a complete PCR, but typically uses only the extension of a primer by a single, fluorescence-labeled dideoxyribonucleic acid molecule (ddNTP) that is complementary to the nucleotide to be investigated. The nucleotide at the polymorphic site can be identified via detection of a primer that has been extended by one base and is fluorescently labeled (e.g., Kobayashi et al, Mol. Cell. Probes, 9:175-182, 1995).

Amplification products generated using an amplification reaction (e.g., PCR) can also be analyzed by the use of denaturing gradient gel electrophoresis. Different alleles can be identified based on the different sequence-dependent melting properties and electrophoretic migration of DNA in solution (see, e.g., Erlich, ed., PCR Technology, Principles and Applications for DNA Amplification, W. H. Freeman and Co, New York, 1992, Chapter 7).

In other embodiments, alleles of target sequences can be differentiated using single-strand conformation polymorphism analysis, which identifies base differences by alteration in electrophoretic migration of single stranded PCR products, as described, e.g, in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to sequence differences between alleles of target regions.

The presence or absence of a mutation (e.g., a nucleic acid mutation) can be detected using allele-specific amplification or primer extension methods. These reactions typically involve use of primers that are designed to specifically target the mutant (or wildtype) site via a mismatch at the 3′ end of a primer, e.g., at the 3′ nucleotide or penultimate 3′ nucleotide. The presence of a mismatch effects the ability of a polymerase to extend a primer when the polymerase lacks error-correcting activity. For example, to detect a V600E mutant sequence using an allele-specific amplification- or extension-based method, a primer complementary to the mutant A allele at nucleotide position 1799 in codon 600 of BRAF is designed such that the 3′ terminal nucleotide hybridizes at the mutant position. The presence of the mutant allele can be determined by the ability of the primer to initiate extension. If the 3′ terminus is mismatched, the extension is impeded. Thus, for example, if a primer matches the mutant allele nucleotide at the 3′ end, the primer will be efficiently extended. Amplification may also be performed using an allele-specific primer that is specific from the BRAF wildtype sequence at position 1799.

Typically, the primer is used in conjunction with a second primer in an amplification reaction. The second primer hybridizes at a site unrelated to the mutant position. Amplification proceeds from the two primers leading to a detectable product signifying the particular allelic form is present. Allele-specific amplification- or extension-based methods are described in, for example, WO 93/22456; U.S. Pat. Nos. 5,137,806; 5,595,890; 5,639,611; and U.S. Pat. No. 4,851,331.

Using allele-specific amplification-based genotyping, identification of the alleles requires only detection of the presence or absence of amplified target sequences. Methods for the detection of amplified target sequences are well known in the art. For example, gel electrophoresis and probe hybridization assays described are often used to detect the presence of nucleic acids.

In an alternative probe-less method, the amplified nucleic acid is detected by monitoring the increase in the total amount of double-stranded DNA in the reaction mixture, is described, e.g., in U.S. Pat. No. 5,994,056; and European Patent Publication Nos. 487,218 and 512,334. The detection of double-stranded target DNA relies on the increased fluorescence various DNA-binding dyes, e.g., SYBR Green, exhibit when bound to double-stranded DNA.

As appreciated by one in the art, allele-specific amplification methods can be performed in reactions which employ multiple allele-specific primers to target particular alleles. Primers for such multiplex applications are generally labeled with distinguishable labels or are selected such that the amplification products produced from the alleles are distinguishable by size. Thus, for example, both wildtype and mutant V600E alleles in a single sample can be identified using a single amplification reaction by gel analysis of the amplification product.

An allele-specific oligonucleotide primer may be exactly complementary to one of the alleles in the hybridizing region or may have some mismatches at positions other than the 3′ terminus of the oligonucleotide. For example the penultimate 3′ nucleotide may be mismatched in an allele-specific oligonucleotide. In other embodiments, mismatches may occur at (nonmutant) sites in both allele sequences.

In some embodiments, allele-specific hybridization is performed in an assay format using an immobilized target or immobilized probe. Such formats are known in the art and include, e.g., dot-blot formats and reverse dot blot assay formats are described in U.S. Pat. Nos. 5,310,893; 5,451,512; 5,468,613; and 5,604,099; each incorporated herein by reference.

4. RNA-Seq

RNA-Seq, also called Whole Transcriptome Shotgun Sequencing (WTSS) refers to the use of high-throughput sequencing technologies to sequence cDNA in order to get information about a sample's RNA content. Publications describing RNA-Seq include: Wang et al. “RNA-Seq: a revolutionary tool for transcriptomics” Nature Reviews Genetics 10 (1): 57-63 (January 2009); Ryan et al. BioTechniques 45 (1): 81-94 (2008); and Maher et al. “Transcriptome sequencing to detect gene fusions in cancer”. Nature 458 (7234): 97-101 (January 2009).

5. Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GENCHIP™ technology, or Incyte's microarray technology.

The development of microarray methods for large-scale analysis of gene expression makes it possible to search systematically for molecular markers of cancer classification and outcome prediction in a variety of tumor types.

6. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

7. MassARRAY Technology

The MassARRAY (Sequenom, San Diego, Calif.) technology is an automated, high-throughput method of gene expression analysis using mass spectrometry (MS) for detection. According to this method, following the isolation of RNA, reverse transcription and PCR amplification, the cDNAs are subjected to primer extension. The cDNA-derived primer extension products are purified, and dispensed on a chip array that is pre-loaded with the components needed for MALTI-TOF MS sample preparation. The various cDNAs present in the reaction are quantitated by analyzing the peak areas in the mass spectrum obtained.

8. Immunohistochemistry

Immunohistochemistry (“IHC) methods are also suitable for detecting the expression levels of the biomarkers of the present invention Immunohistochemical staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Thus, antibodies or antisera, preferably polyclonal antisera, and most preferably monoclonal antibodies specific for each marker are used to detect expression. As discussed in greater detail below, the antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody Immunohistochemistry protocols and kits are well known in the art and are commercially available.

Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromagenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Colloidal gold particles.

(c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE® and SPECTRUM GREEN® and/or derivatives of any one or more of the above. The fluorescent labels can be conjugated to the antibody using the techniques disclosed in Current Protocols in Immunology, supra, for example. Fluorescence can be quantified using a fluorimeter.

(d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al. Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor [e.g., orthophenylene diamine (OPD) or 3,3′,5,5′-tetramethyl benzidine hydrochloride (TMB)]. 3,3-Diaminobenzidine (DAB) may also be used to visualize the HRP-labeled antibody;

(ii) alkaline phosphatase (AP) with para-Nitrophenyl phosphate as chromogenic substrate; and

(iii) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice verse. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired. For example, epitope retrieval methods, such as heating the tissue sample in citrate buffer may be carried out [see, e.g., Leong et al. Appl. Immunohistochem. 4(3):201 (1996)].

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation.

The extent of binding of antibody to the sample is determined by using any one of the detectable labels discussed above. Preferably, the label is an enzymatic label (e.g. HRPO) which catalyzes a chemical alteration of the chromogenic substrate such as 3,3′-diaminobenzidine chromogen. Preferably the enzymatic label is conjugated to antibody which binds specifically to the primary antibody (e.g. the primary antibody is rabbit polyclonal antibody and secondary antibody is goat anti-rabbit antibody).

Specimens thus prepared may be mounted and coverslipped. Slide evaluation is then determined, e.g. using a microscope.

IHC may be combined with morphological staining, either prior to or thereafter. After deparaffinization, the sections mounted on slides may be stained with a morphological stain for evaluation. The morphological stain to be used provides for accurate morphological evaluation of a tissue section. The section may be stained with one or more dyes each of which distinctly stains different cellular components. In one embodiment, hematoxylin is use for staining cellular nucleic of the slides. Hematoxylin is widely available. An example of a suitable hematoxylin is Hematoxylin II (Ventana). When lighter blue nuclei are desired, a bluing reagent may be used following hematoxylin staining. One of skill in the art will appreciate that staining may be optimized for a given tissue by increasing or decreasing the length of time the slides remain in the dye.

Automated systems for slide preparation and IHC processing are available commercially. The Ventana® BenchMark XT system is an example of such an automated system.

After staining, the tissue section may be analyzed by standard techniques of microscopy. Generally, a pathologist or the like assesses the tissue for the presence of abnormal or normal cells or a specific cell type and provides the loci of the cell types of interest. Thus, for example, a pathologist or the like would review the slides and identify normal cells (such as normal lung cells) and abnormal cells (such as abnormal or neoplastic lung cells). Any means of defining the loci of the cells of interest may be used (e.g., coordinates on an X-Y axis).

Anti-c-met antibodies suitable for use in IHC are well known in the art, and include SP-44 (Ventana), DL-21 (Upstate), MET4, ab27492 (Abcam), PA1-37483 (Pierce Antibodies). One of ordinary skill understands that additional suitable anti-c-met antibodies may be identified and characterized by comparing with c-met antibodies using the IHC protocol disclosed herein, for example. Anti-phospho-c-met antibodies are known in the art and include anti-phospho-c-met antibody Y1234/5 from Cell Signalling Technologies. Anti-HGF antibodies suitable for use in IHC are also well-known in the art, and include: ab24865 (Abcam), H00003082-A01 (Abnova), MA1-24767 (Thermo Fisher), LS-C123743 (Life Span). As used herein, it is understood that detection of HGF in a sample of the patient's tumor encompasses, for example, detection of HGF in tumor stroma present in a sample of the patient's tumor as well as detection of HGF in tumor cells. Assays (such as ELISA assays) for detection of HGF in serum are commercially available and known in the art. See e.g., Catenacci et al, Cancer Discovery (2011) 1:573.

In some embodiments, control cell pellets with various staining intensities may be utilized as controls for IHC analysis as well as scoring controls. For example, H441 (strong c-met staining intensity); A549 (moderate c-met staining intensity); H1703 (weak c-met staining intensity), HEK-293 (293) (weak c-met staining intensity); and TOV-112D (negative c-met staining intensity) or H1155 (negative c-met staining intensity).

In some embodiments, c-met staining intensity criteria may be evaluated according to Table A:

TABLE A

| IHC score | Staining criteria |
|---|---|
| 0 | samples with negative or equivocal staining, or <50% tumor |
| | cells with weak (1+) or combined weak (1+) & moderate |
| | (2+) staining |
| 1 | 50% or more tumor cells with weak (1+) or combined weak |
| | (1+) & moderate (2+) staining, but less than 50% tumor cells |
| | with moderate (2+) or combined moderate (2+) & strong |
| | (3+) staining |
| 2 | 50% or more tumor cells with moderate (2+) or combined |
| | moderate (2+) & strong (3+) staining, but less than 50% |
| | tumor cells with strong (3+) staining |
| 3 | 50% or more tumor cells with strong (3+) staining |

In some embodiments, “clinical Met diagnostic positive” and “clinical Met diagnostic negative” categories are defined as follows:

Clinical c-met diagnostic positive: IHC score 2 or 3 (as defined in Table A), and

Clinical c-met diagnostic negative: IHC score 0 or 1 (as defined in Table A).

In some embodiments, high c-met biomarker associated is an IHC score of 2, an IHC score of 3, or an IHC score of 2 or 3. In some embodiments, low c-met biomarker is an IHC score of 0, an IHC score of 1 or an IHC score of 0 or 1.

9. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

10. Gene Amplification

Detecting amplification of the c-met gene is achieved using certain techniques known to those skilled in the art. For example, comparative genome hybridization may be used to produce a map of DNA sequence copy number as a function of chromosomal location. See, e.g., Kallioniemi et al. (1992) Science 258:818-821. Amplification of the c-met gene may also be detected, e.g., by Southern hybridization using a probe specific for the c-met gene or by real-time quantitative PCR.

In certain embodiments, detecting amplification of the c-met gene is achieved by directly assessing the copy number of the c-met gene, for example, by using a probe that hybridizes to the c-met gene. For example, a FISH assay may be performed. In certain embodiments, detecting amplification of the c-met gene is achieved by indirectly assessing the copy number of the c-met gene, for example, by assessing the copy number of a chromosomal region that lies outside the c-met gene but is co-amplified with the c-met gene. Biomarker expression may also be evaluated using an in vivo diagnostic assay, e.g. by administering a molecule (such as an antibody) which binds the molecule to be detected and is tagged with a detectable label (e.g. a radioactive isotope) and externally scanning the patient for localization of the label.

11. Other Exemplary Methods

The biomarker can be detected by a variety of immunoassay methods (including IHC, described herein, e.g., supra). For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Ten, eds., 7th ed. 1991).

Commonly used assays include noncompetitive assays, e.g., sandwich assays, and competitive assays. Typically, an assay such as an ELISA assay can be used. Elisa assays are known in the art, e.g., for assaying a wide variety of tissues and samples, including plasma or serum. An ELISA assay for assaying HGF in serum is exemplified herein. Anti-HGF antibodies suitable for use in ELISA are known in the art.

A wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker. Sandwich assays are commonly used assays. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface may be glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by a labeled reporter molecule.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, and other are discussed herein. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest Immunofluorescence and EIA techniques are both very well established in the art and are discussed herein.

Other detection techniques, e.g., MALDI, may be used to directly detect the presence of biomarker, e.g., mutant Braf, in a sample.

### V. Articles of Manufacture

In another embodiment of the invention, an article of manufacture for use in treating cancer (such as melanoma or papillary thyroid carcinoma) is provided. The article of manufacture comprises a container and a label or package insert on or associated with the container. Suitable containers include, for example, bottles, vials, syringes, etc. The containers may be formed from a variety of materials such as glass or plastic. The container holds or contains a composition comprising the cancer medicament as the active agent and may have a sterile access port (for example the container may be an intravenous solution bag or a vial having a stopper pierceable by a hypodermic injection needle).

The article of manufacture may further comprise a second container comprising a pharmaceutically-acceptable diluent buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

The article of manufacture of the present invention also includes information, for example in the form of a package insert, indicating that the composition is used for treating cancer based on expression level of the biomarker(s) herein. The insert or label may take any form, such as paper or on electronic media such as a magnetically recorded medium (e.g., floppy disk) or a CD-ROM. The label or insert may also include other information concerning the pharmaceutical compositions and dosage forms in the kit or article of manufacture. Methods include any treatment and diagnostic methods herein.

According to one embodiment of the invention, an article of manufacture is provided comprising, packaged together, a c-met antagonist (e.g., an anti-c-met antibody) in a pharmaceutically acceptable carrier and a package insert indicating that the c-met antagonist is for treating a patient with cancer (such as melanoma) based on expression of a c-met biomarker. In some embodiment, the treatment is in combination with a B-raf antagonist. In some embodiments, the package insert indicates that the c-met antagonist is combined with a B-raf antagonist for treating a patient with cancer (such as melanoma) based on expression of a c-met biomarker and a B-raf biomarker. In some embodiments, B-raf biomarker is B-raf V600E.

The invention also concerns a method for manufacturing an article of manufacture comprising combining in a package a pharmaceutical composition comprising a c-met antagonist (e.g., an anti-c-met antibody) and a package insert indicating that the pharmaceutical composition is for treating a patient with cancer (such as NSCLC) based on expression of a c-met biomarker. In some embodiment, the treatment is in combination with a B-raf antagonist. In some embodiments, the package insert indicates that the c-met antagonist is combined with a B-raf antagonist for treating a patient with cancer (such as melanoma) based on expression of a c-met biomarker and a B-raf biomarker. In some embodiments, B-raf biomarker is B-raf V600. In some embodiments, B-raf biomarker is B-raf V600E.

The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

### VI. Diagnostic Kits

The invention also concerns diagnostic kits useful for detecting any one or more of the biomarker(s) identified herein. Accordingly, a diagnostic kit is provided which comprises one or more reagents for determining expression of one or more of c-met, and B-raf, such as B-raf V600 biomarker in a sample from a cancer patient. Optionally, the kit further comprises instructions to use the kit to select a cancer medicament (e.g. a c-met antagonist, such as an anti-c-met antibody, in combination with a B-raf antagonist) for treating the cancer patient if the patient expresses the c-met biomarker and/or if the patient expresses the B-raf biomarker. In some embodiments, B-raf biomarker is B-raf V600. In some embodiments, B-raf biomarker is detected using a method comprising (a) performing PCR or sequencing on nucleic acid (e.g., DNA) extracted from a sample of the patient's melanoma; and (b) determining expression of BRAF^{V600} in the sample. In some embodiments, the melanoma sample is formalin-fixed paraffin-embedded. In some embodiments, c-met biomarker is HGF and expression is detected in a sample of the patient's melanoma (or melanoma stroma) using IHC. In some embodiments, c-met biomarker is HGF and expression is detected in a sample of the patient's serum using ELISA. Diagnostic methods include any diagnostic methods herein.

### VII. Methods of Advertising

The invention herein also concerns a method for advertising a cancer medicament comprising promoting, to a target audience, the use of the cancer medicament (e.g. anti-c-met antibody) for treating a patient with cancer based on expression of c-met biomarker and/or B-raf biomarker.

Advertising is generally paid communication through a non-personal medium in which the sponsor is identified and the message is controlled. Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the diagnostic method herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store PA systems, or anywhere a visual or audible communication can be placed.

More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments and diagnostics. The advertising may be individualized or customized based on user characterizations defined by service interaction and/or other data such as user demographics and geographical location.

### EXAMPLES

### Example 1

### Growth Factor-Driven Resistance to Anti-Cancer Kinase Inhibitors Methods

RTK Ligand Matrix Screen.

Cell viability was assessed using the nucleic acid stain Syto 60 (Invitrogen). Cells (3000-5000 per well) were seeded into 96 well plates and allowed to adhere overnight. The next day, cells were treated with (or without) 50 ng/mL RTK ligand and concomitantly exposed to an increasing concentration range of the relevant kinase inhibitor. Following 72 hours drug exposure, cells were fixed in 4% formaldehyde, stained with Syto 60 and cell number was assessed using an Odyssey scanner (Li-Cor). Cell viability was calculated by dividing the fluorescence obtained from the drug-treated cells by the fluorescence obtained from the control (no drug) treated cells.

Cell lines. Human cancer cell lines were obtained and tested for sensitivity using an automated platform as previously described (Johannessen, C. M. et al. COT drives resistance to RAF inhibition through MAP kinase pathway reactivation. Nature 468, 968-972, doi:10.1038/nature09627 (2010)). Cell lines were maintained at 37° C. in a humidified atmosphere at 5% CO₂ and grown in RPMI 1640 or DMEM/F12 growth media (GIBCO) supplemented with 10% fetal bovine serum (GIBCO), 50 units/mL penicillin and 50 μg/mL streptomycin (GIBCO).

Reagents.

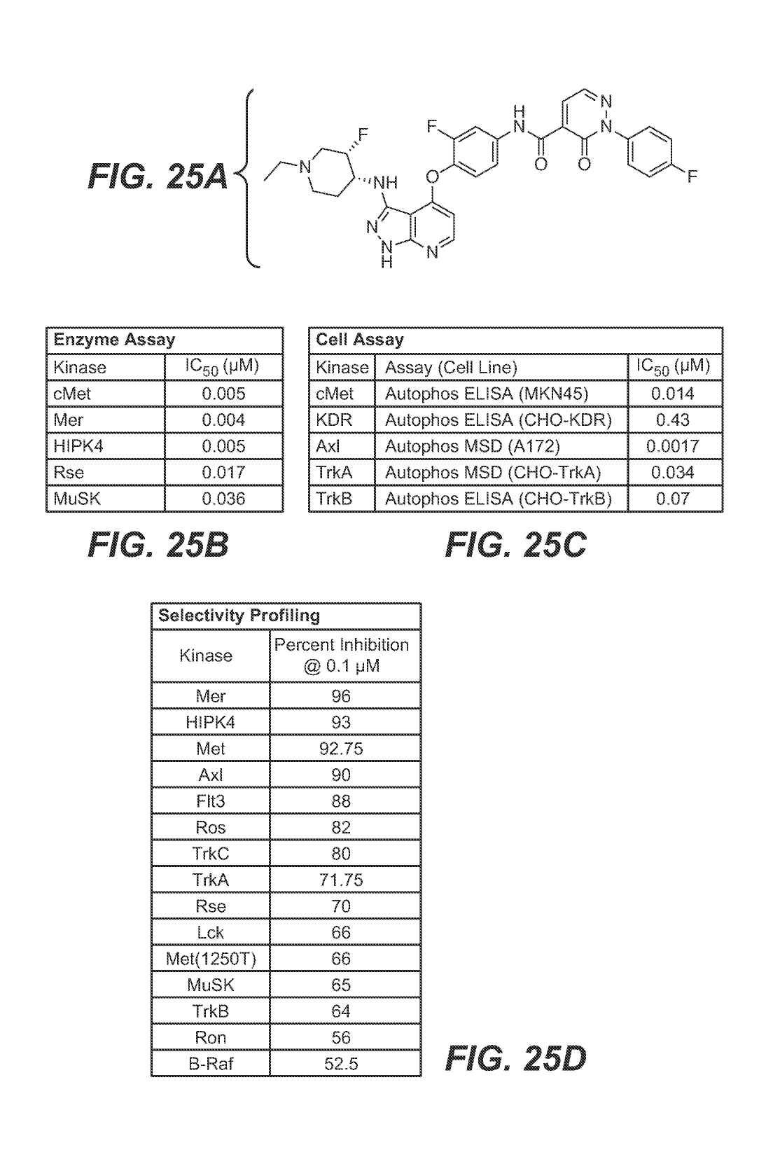

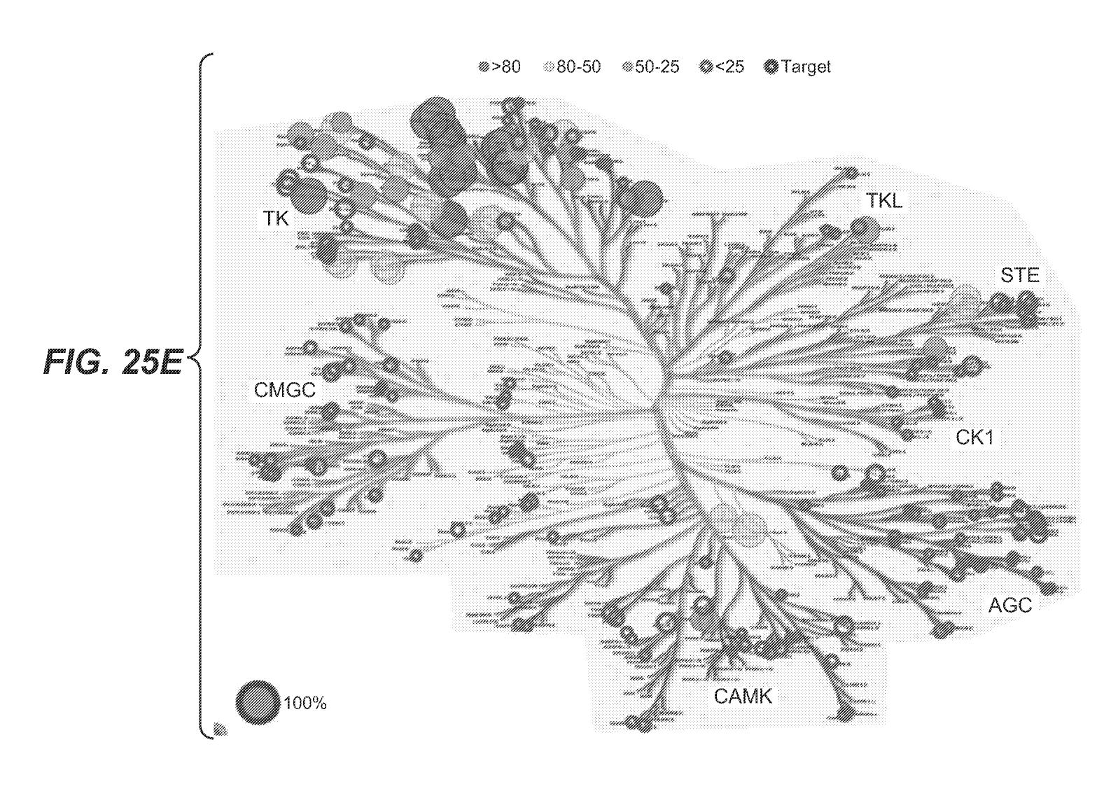

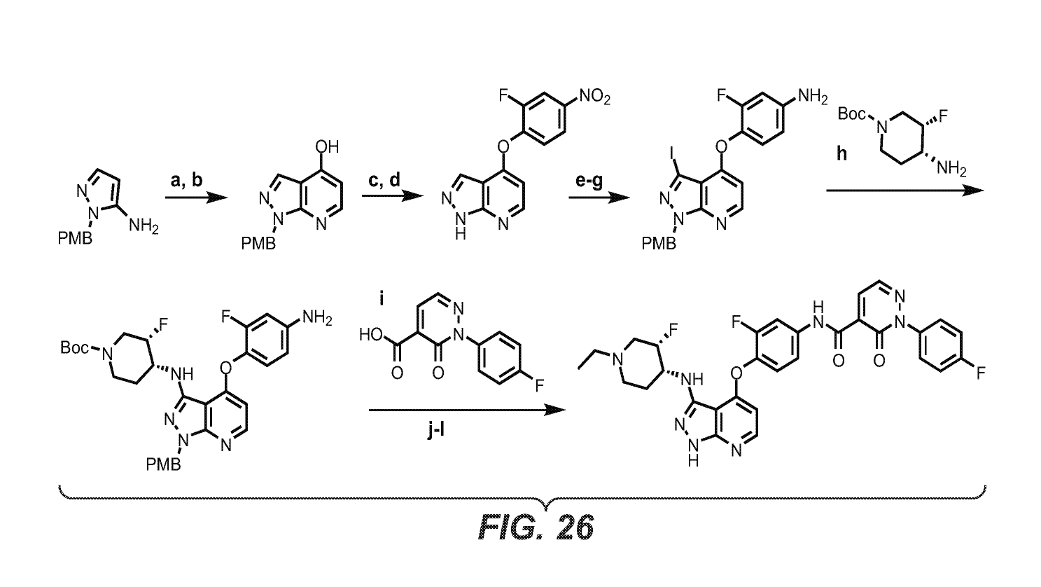

Lapatinib, sunitinib and erlotinib were purchased from LC Laboratories. Crizotinib, TAE684, AZD6244 and BEZ235 were purchased from Selleck Chemicals. PD173074 was purchased from Tocris Bioscience. PLX4032 was purchased from Active Biochem. Recombinant human (rh) HGF, EGF, FGF-basic, IGF-1 and PDGF-AB were purchased from Peprotech. rhNRG1-β1 was purchased from R and D Systems. For in vivo studies, 3D6 anti-MET agonist antibody, RG7204 (PLX4032) and GDC-0712 were generated at Genentech. GDC-0712 was used in xenograft experiments as it has a similar kinase profile as crizotinib (Liederer, B. M. et al. Xenobiotica 41, 327-339, doi:10.3109/00498254. 2010.542500 (2011))(FIGS. 25 and 26).

Immunoblotting.

Cell lysates were harvested using Nonidet-P40 lysis buffer, supplemented with Halt protease and phosphatase inhibitor cocktail (Thermo Scientific) and immunodetection of proteins was carried out using standard protocols. The phospho-HER2 (Y1248; #2247), HER2 (#2242), phospho-HER3 (Y1289; #4791), phospho-MET (Y1234/5; #3126), PDGFRα (#5241), phospho-FRS2α (Y196; #3864), IGF-1Rβ (#3027), phospho-ALK (Y1604; #3341), AKT (#9272), phospho-ERK (T202/Y204; #9101), ERK (#9102), GAPDH (#2118) and β-Tubulin (#2146) antibodies were purchased from Cell Signaling Technologies. Antibodies to HER3 (SC-285), MET (SC-10), phospho-PDGFRα (SC-12911), FRS2α (SC-8318), FGFR1 (SC-7945), FGFR2 (SC-122), FGFR3 (SC-13121) and ALK (SC-25447) were purchased from Santa Cruz Biotechnologies. Phospho-AKT (S473; #44-621G) antibody was purchased from Invitrogen. Phospho-EGFR (Y1068; ab5644) antibody was purchased from Abeam. EGFR (#610017) antibody was purchased from BD Biosciences. PARP (#14-6666-92) antibody was purchased from eBioscience. Densitometry was carried out using ImageJ software.

Tissue Samples.

Primary breast tumor samples with appropriate IRB approval and patient informed consent were obtained from the following sources: Cureline (South San Francisco, Calif.), ILSbio (Chestertown, Md.) and the Cooperative Human Tissue Network of the National Cancer Institute. Metastatic melanoma tumour samples were obtained from the BRIM2 trial. The human tissue samples used in the study were de-identified (double-coded) prior to their use and thus the study using these samples is not considered human subject research under the US Department of Human and Health Services regulations and related guidance. Immunohistochemistry for MET was performed on formalin-fixed paraffin-embedded sections cut at a thickness of 4 μm on to positively charged glass slides. The staining was performed on a Discovery XT autostainer with Ultraview detection (VMSI, Tucson, Ariz.) using the MET rabbit monoclonal antibody SP44 (Spring BioScience, Pleasanton, Calif.; #M3441) and CC1 standard antigen retrieval. Sections were counterstained with hematoxylin and specific staining (e.g., membraneous staining) for c-MET was scored on a scale from 0 (no staining) to 3+(strong staining).

The scoring scheme is described in co-owned U.S. Patent Publication No. US20120089541A1, the contents of which are herein incorporated by reference in its entirety. Briefly, tumor cells were scored for c-Met staining. The staining was classified as strong (3+), moderate (2+), weak (1+), equivocal (+/−) or negative (−) staining intensity relative to control cell pellets with various staining intensities may be utilized as controls for IHC analysis as well as scoring controls. H441 (strong c-met staining intensity); A549 (moderate c-met staining intensity); H1703 (weak c-met staining intensity), HEK-293 (293) (weak c-met staining intensity); and TOV-112D (negative c-met staining intensity) or H1155 (negative c-met staining intensity) were used. In addition to evaluating staining intensity, percentages of various staining intensities/patterns were visually estimated in the samples with heterogeneous signals.

Hepatocyte Growth Factor (HGF) ELISA.

Plasma was obtained from metastatic melanoma patients pre-dose cycle one and the concentration of HGF in patient-derived plasma were quantitatively measured using a sandwich enzyme-linked immunosorbent assay (ELISA). Wells of NUNC MaxiSorp microtiter plates were coated (ON, 4° C.) with 0.5 μg/mL of affinity-purified Goat antihuman hepatocyte growth factor polyclonal antibody in 100 μL of coating buffer (0.05M sodium carbonate buffer, pH 9.6) and were then blocked with 0.5% bovine serum albumin (BSA) in assay buffer (PBS, 0.5% BSA, 0.05% P 20, 0.25% CHAPS, 0.35M NaCl, 5 mM EDTA, 10 ppm Proclin300, pH 7.4) for 1 hour at room temperature. Diluted human hepatocyte growth factor controls and plasma samples (100 μL) in assay buffer were loaded in duplicates and incubated for 2 hours at room temperature, followed by the addition of 100 μL of affinity-purified goat antihuman hepatocyte growth factor biotin (150 ng/mL) for an additional 1 hour at room temperature. Avidin-conjugated horseradish peroxidase (40 ng/mL) in PBS, 0.5% BSA, 0.05% P 20, 10 ppm Proclin300, pH 7.4, was added (1 hour, room temperature), and the reaction was visualized by the addition of 100 μL of chromogenic substrate (TMB) for 15 minutes. The reaction was stopped with 1M phosphoric acid and absorbance at 450 nm was measured with reduction at 630 nm with an ELISA plate reader. Plates were washed 3 times with washing buffer (0.05% Tween 20/PBS) after each step. As a reference for quantification, a standard curve was established by a serial dilution of human hepatocyte growth factor (CritRS CR67; 2000-15.625 pg/mL).

Xenograft Studies.

All procedures conformed to the guidelines and principles set by the Institutional Animal care and Use Committee of Genentech and were carried out in an AAALAC (Association for the Assessment and Accreditation of Laboratory Animal care) accredited facility. 10 million 928MEL or 624MEL BRAF mutant melanoma cells (suspended in HBSS/Matrigel (e.g., 1:1 mixture) were inoculated in the right flank of CRL C.B-17 SCID.bg mice (Charles River Laboratories). When tumors reached an average volume of 200 mm3, mice (10 per group) were treated with either Control antibody (Anti-gp120; 10 mg/kg once per week; intraperitoneal), 3D6 (anti-MET agonist antibody; 10 mg/kg once per week; intraperitoneal), RG7204 (PLX4032; 50 mg/kg twice daily, periocular), GDC-0712 (MET small molecular inhibitor, 100 mg/kg every day, periocular) as indicated for 4 weeks. Tumors were measure twice weekly using digital calipers (Fred V. Fowler Company, Inc.). Tumor volumes were calculated using the formula (Lx(W×W))/2. A partial response (PR) in this example was defined as a reduction in tumour volume greater than 50% but less than 100%. A complete response (CR) in this example was defined as 100% reduction in tumour volume. Differences between the PLX4032-treated and the PLX4032- and GDC-0712-treated control antibody groups were determined using two-way ANOVA (*=0.0008).

Secreted Factor Screen.

Recombinant purified secreted factors were purchased from Peprotech and R and D Systems as appropriate, and were reconstituted in PBS/0.1% BSA. Secreted factors were transferred into 96 well plates at a concentration of 1 μg/mL, and subsequently diluted to 100 ng/mL in media containing either no drug or 5 μM PLX4032. Equal volumes of diluted factor (final concentration 50 ng/mL) were arrayed into the 384 well plates pre-seeded with SK-MEL-28 cells (500 cells per wells seeded the day before) using an Oasis liquid handler. Following 72 h incubation, cell viability was determined using Cell Titer Glo (Promega).

Statistics.

Error bars in cell viability assays represent mean plus or minus standard error of the mean (s.e.m.). For correlation of receptor with ligand rescue was carried out using a 2×2 contingency table with the following groups: receptor positive, RTK ligand rescued; receptor positive, RTK ligand non-rescued; receptor negative, RTK ligand rescued; receptor negative, RTK ligand non-rescued. Significance was determined using a two-tailed Fisher Exact Probability Test.

Statistical Analysis of BRIM2 Clinical Samples.

HGF levels were log-transformed, and the Kolmogorov-Smirnoff test was used to test the resulting distribution for departure from the Gaussian distribution. The Cox-proportional model was used to test the log-transformed HGF levels for association with the progression free survival (PFS) and overall survival (OS). Association between the response and HGF levels was tested using the Wilcoxon rank-sum test. Kaplan-Meier (KM) curves were used to show display the relationship between the HGF levels and the time-to-event outcomes (PFS and OS). The number of events/patients and medium time to event is shown for each group. The cox-proportional model of the outcome as the function of the continuous HGF level was used to calculate the hazard ratio and corresponding p-value.

### Results

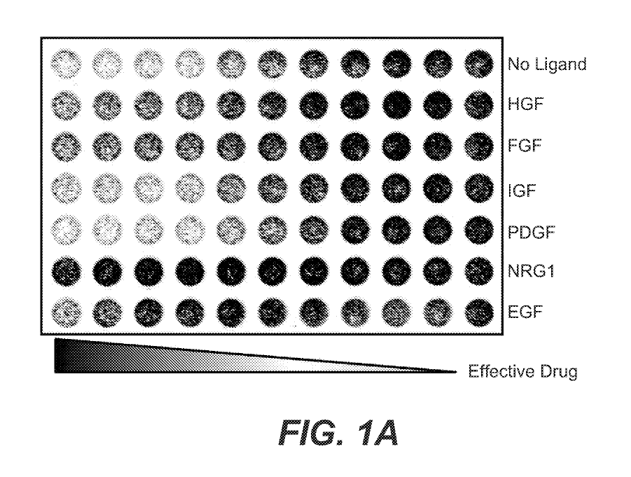

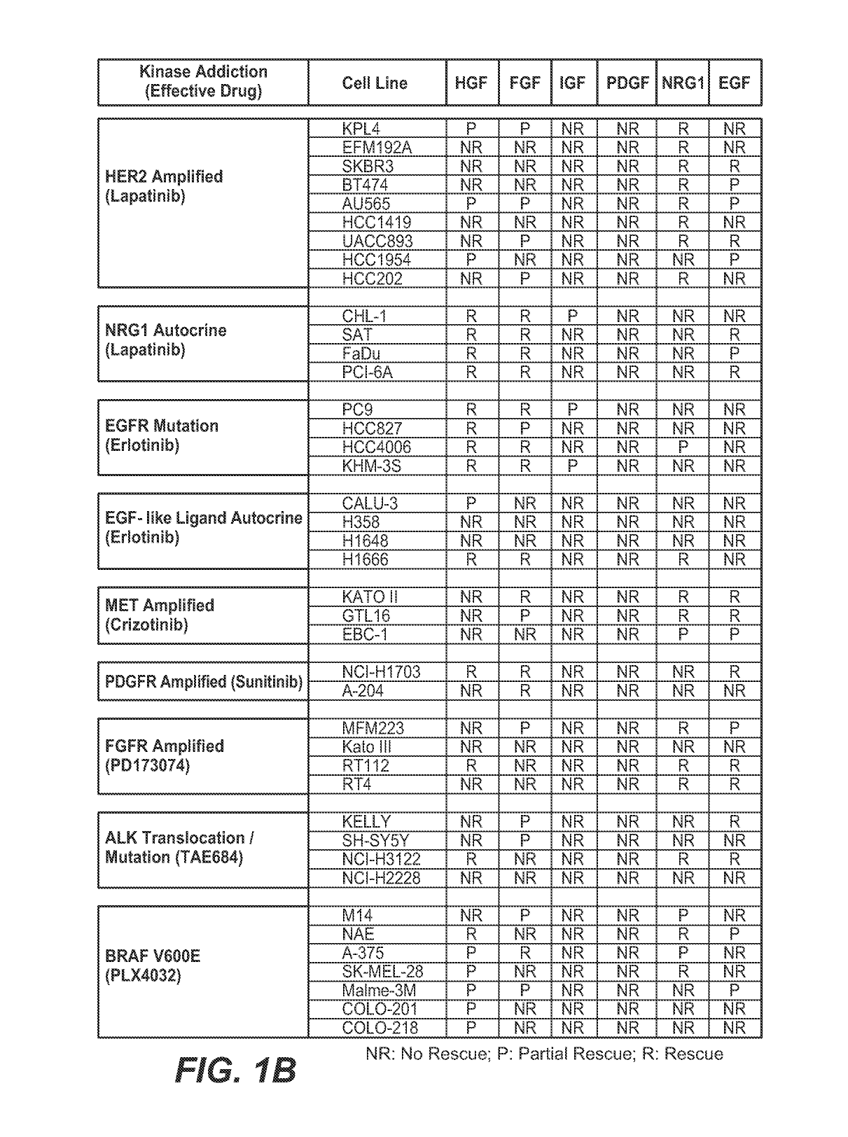

Using 41 different human tumor-derived cell lines with previously defined kinase dependency⁷⁻⁹, we undertook a “matrix analysis” to examine the effects of 6 different RTK ligands (HGF, EGF, FGF, PDGF, NRG1, IGF)—known to be widely expressed in cancer cells and tumor stroma¹⁰—on drug response. Specifically, we quantified the effect of exposing these cancer cell lines (e.g., AU565 (HER2 amp)) to each ligand on the IC50 for a kinase inhibitor (e.g., lapatinib) that otherwise potently suppresses their growth within 72 hours (FIG. 1A). Nearly all of the kinase-dependent cancer cell lines tested, which included cells derived from multiple tissue types and with distinct kinase dependencies (EGFR, HER2, BRAF, MET, ALK, PDGFR, and FGFR), could be rescued from drug-induced growth inhibition by one or more RTK ligands, highlighting the potentially broad contribution of these ligands to the response to selective kinase inhibitors in kinase-addicted tumor cells (FIG. 1B).

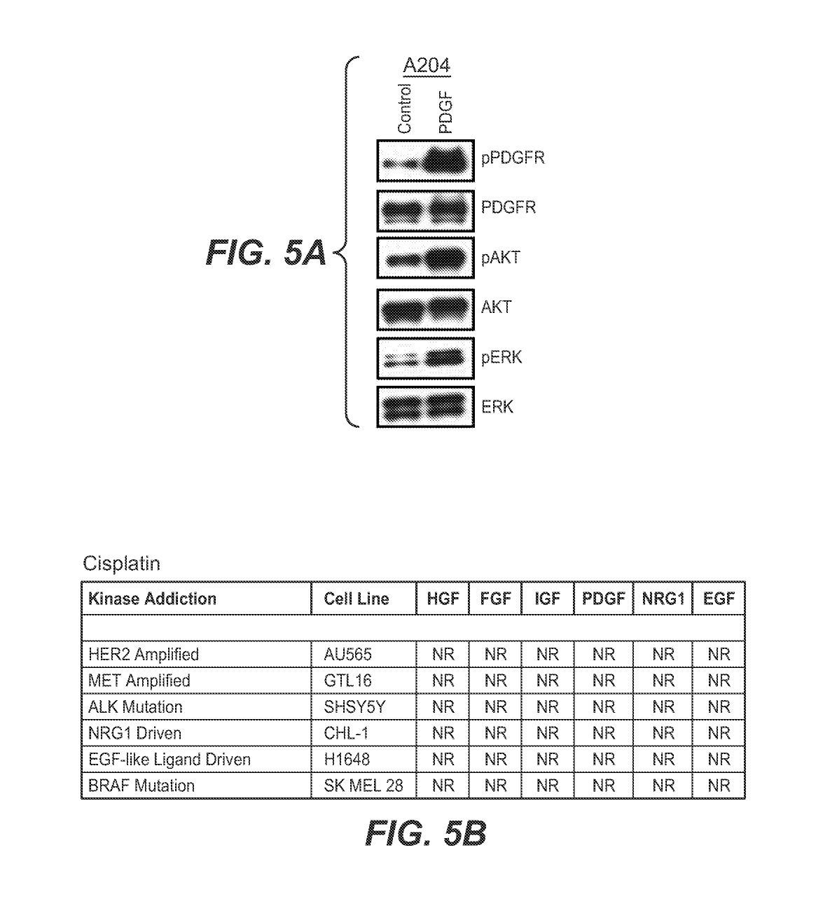

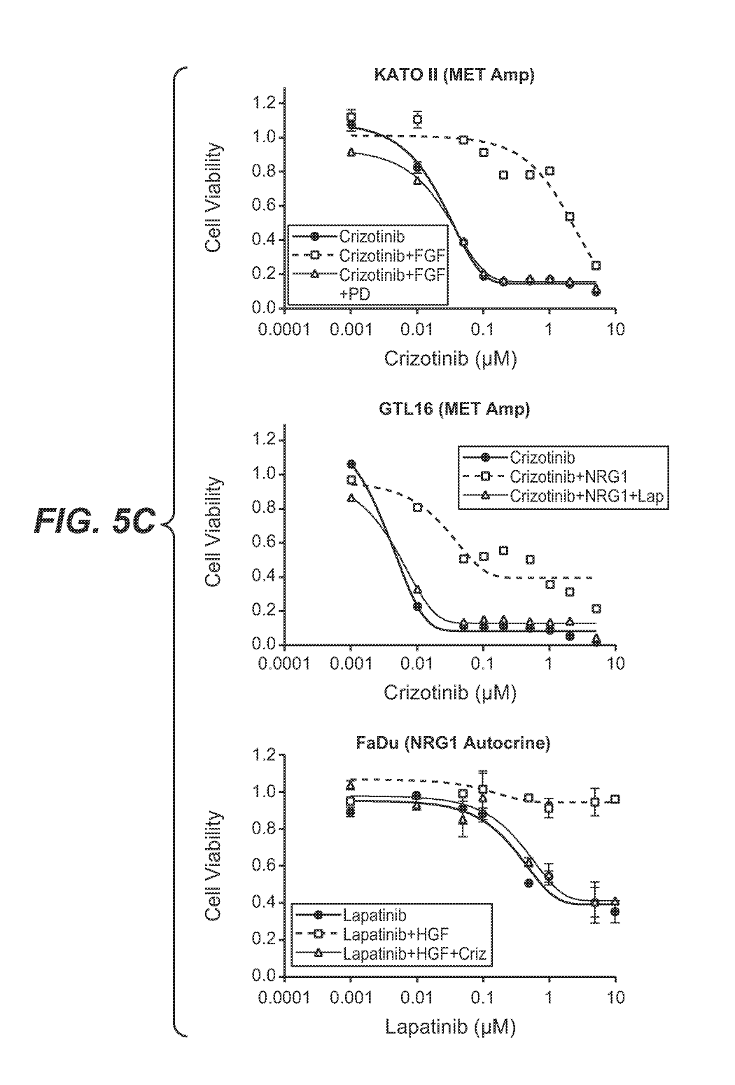

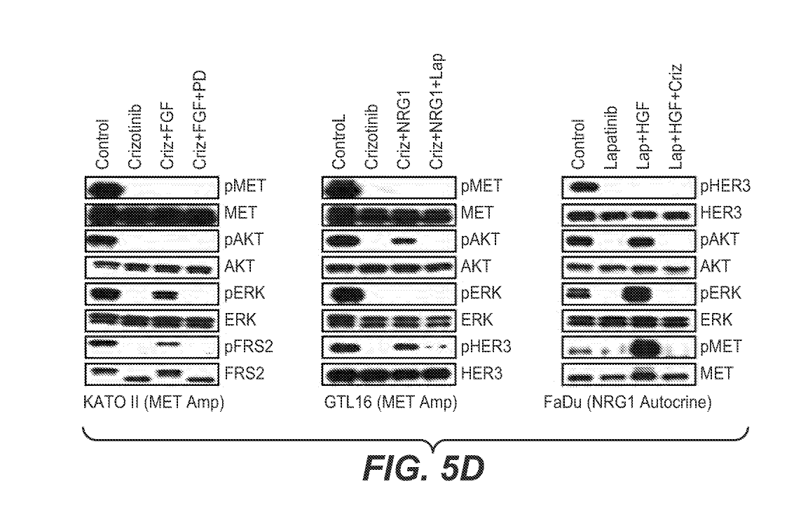

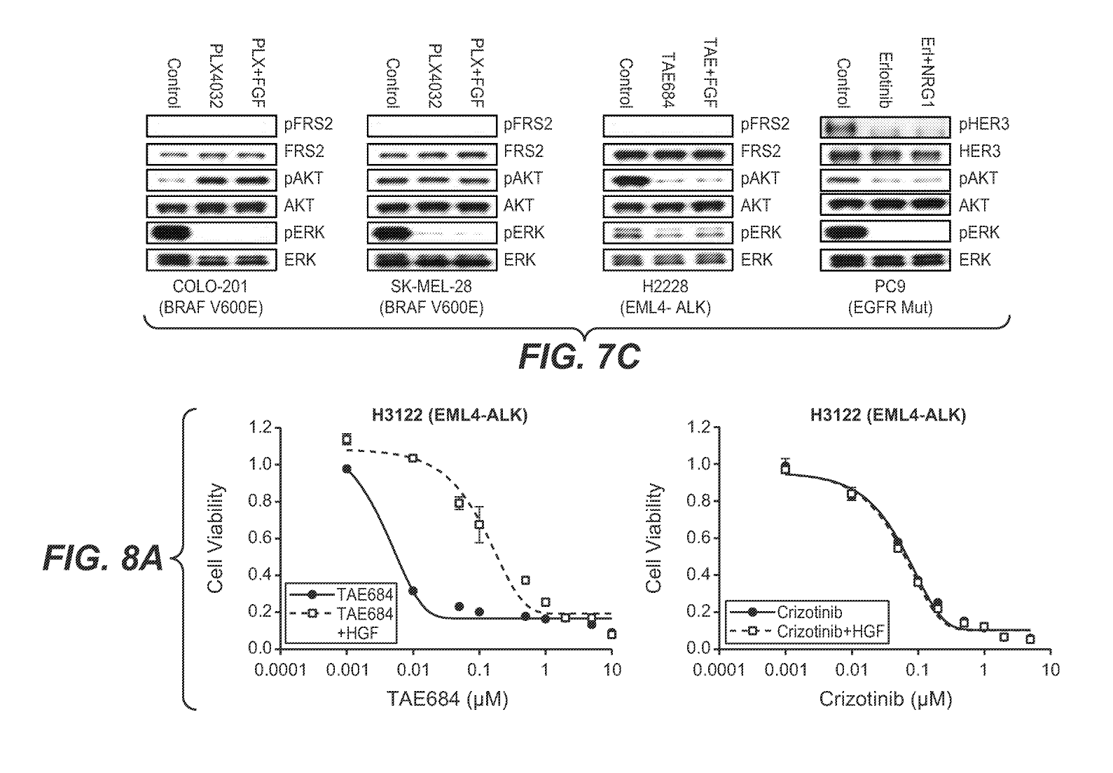

The consequences of ligand exposure on drug response could be categorized in three classes (FIG. 1C); “No rescue”: the addition of ligand did not detectably affect drug response; “Partial rescue”: the ligand partially abrogated treatment response, or “Complete rescue”: the ligand “right-shifted” the IC50 curve>10-fold, or completely suppressed drug response. HGF, FGF and NRG1 were the most broadly active ligands with respect to conferring drug resistance, followed by EGF; whereas, IGF and PDGF had relatively little effect, despite their ability to activate their corresponding receptors (FIG. 5A and FIG. 7A). Notably, many of the tested cell lines could be rescued from treatment sensitivity by exposure to two or even three different ligands, highlighting the apparent capacity of such cells to engage redundant survival pathways upon exposure to a variety of RTK ligands. Significantly, none of the tested RTK ligands could rescue cells from the growth suppressive effects of the chemotherapy drug cisplatin in several tested cell lines, suggesting that the observed ligand rescue effects do not reflect broad protection from generally toxic agents, but rather, are limited to pathway-specific signal disruption (FIG. 5B).

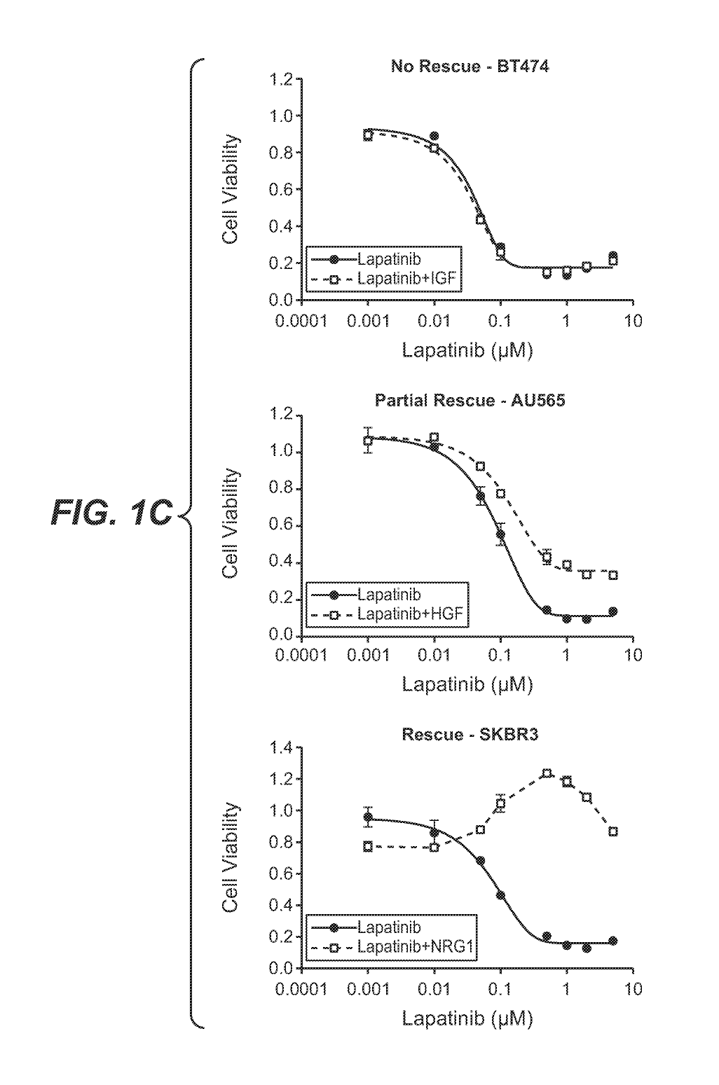

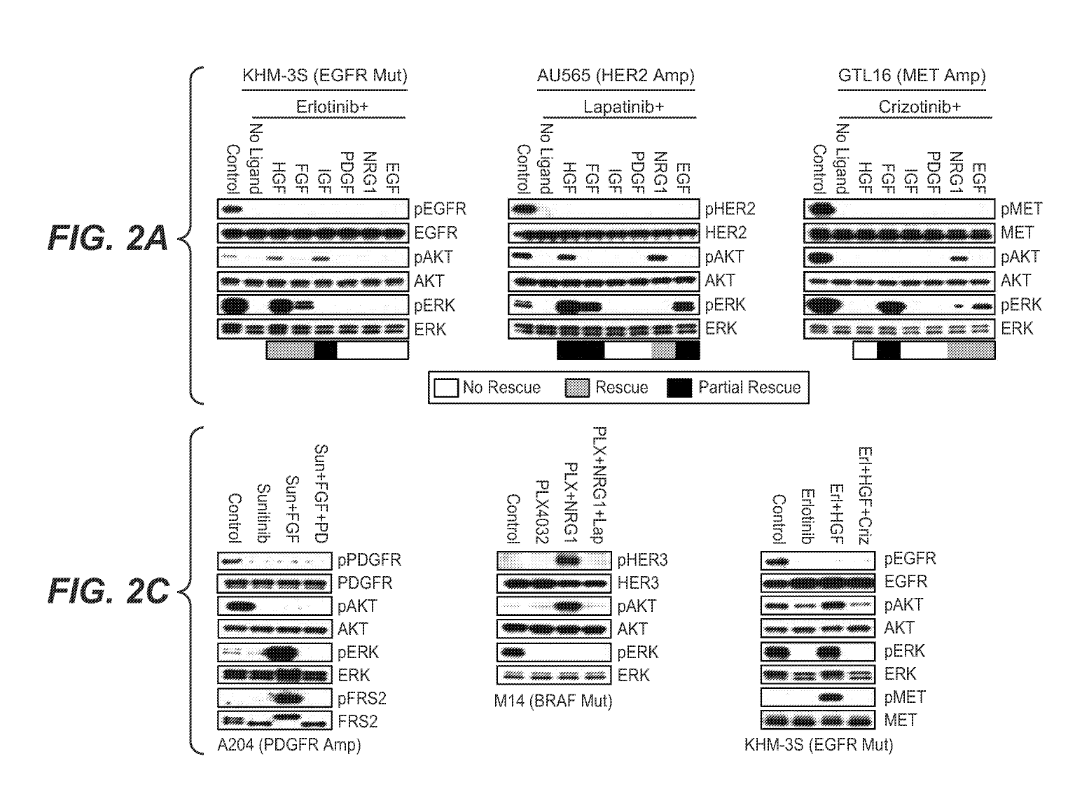

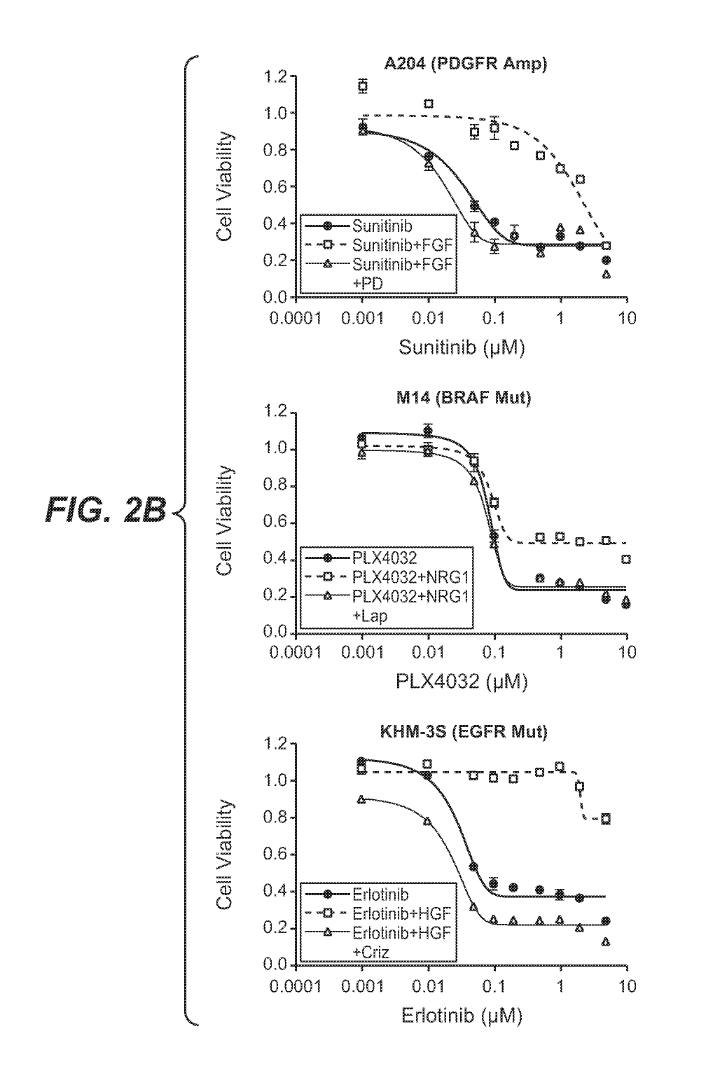

To further explore the signalling dynamics associated with ligand-mediated rescue from kinase dependency, we assessed the status of two critical downstream survival signalling pathways commonly engaged by RTKs—the PI3K/AKT and MAPK/ERK pathways¹¹. In cases where ligand-mediated rescue was achieved, the RTK ligand could efficiently “re-activate” at least one of these pathways despite the presence of the kinase inhibitor (FIG. 2A). Pathway re-activation was not due to re-activation of the oncogenic kinase, as autophosphorylation of the addicting kinase remained suppressed following RTK ligand co-treatment. In the various tested models, HGF re-activated both PI3K and MAPK pathways, IGF and NRG1 only re-activated the PI3K and FGF and EGF only re-activated the MAPK pathway.

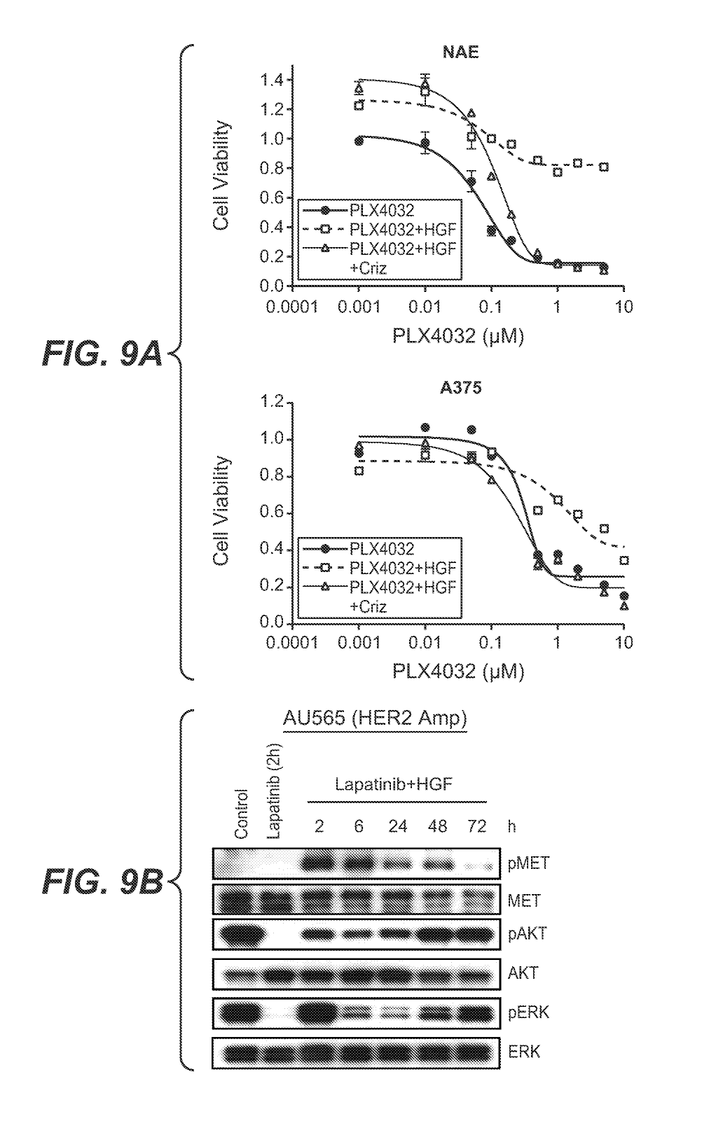

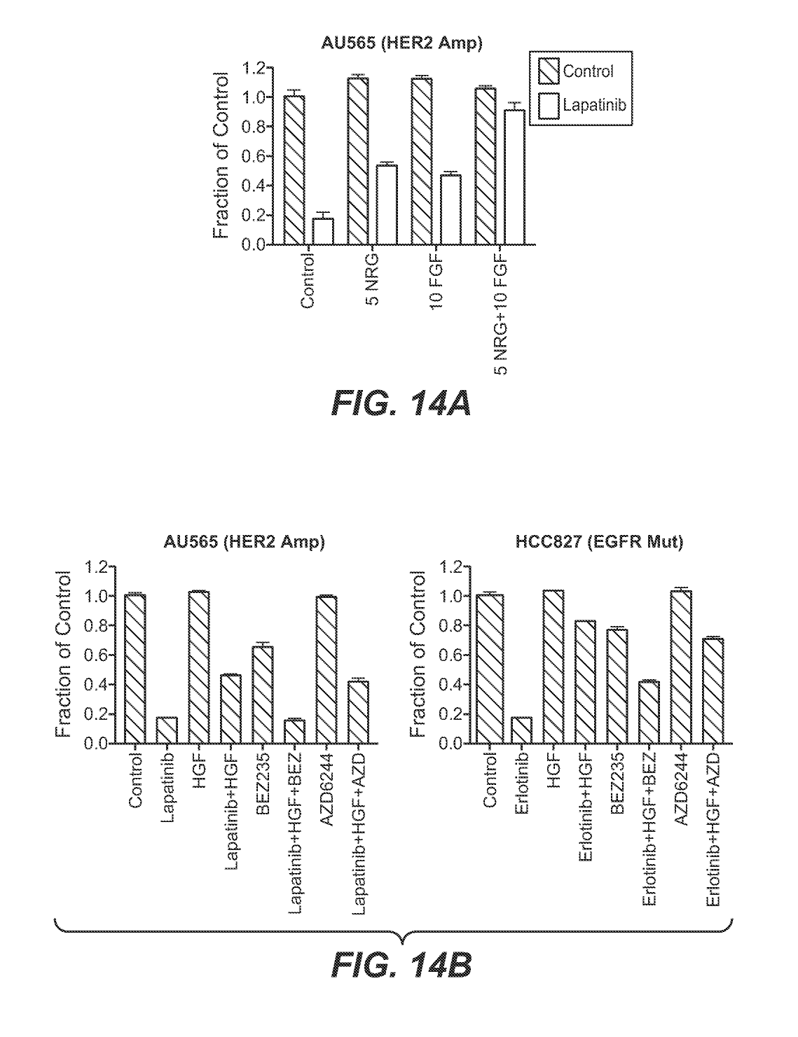

Activation of the “redundant RTK” and consequent downstream survival signalling persisted for at least 48 hours as demonstrated with AU565 cells co-treated with lapatinib and HGF (FIG. 9B). An “additive” role for re-activation of both the PI3K and MAPK pathways was observed in lapatinib-treated AU565 cells in the presence of NRG1, FGF or the combination (FIG. 14A). However, specifically inhibiting the PI3K pathway (and not MAPK) attenuated HGF-promoted drug resistance, which was associated with was associated with engagement of both survival pathways (FIG. 14B).

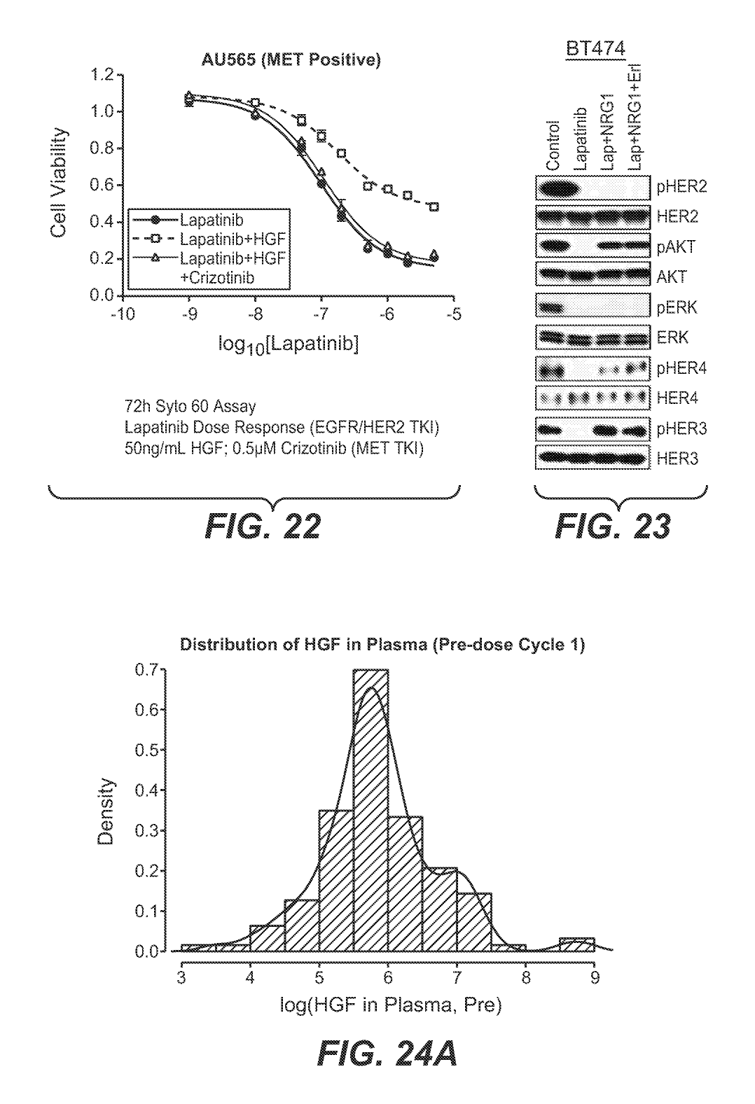

As expected, the observed RTK ligand-induced rescue of cell survival and pathway signalling could be reversed by co-targeting the secondary activated kinase, confirming that the effective ligands were acting via their cognate RTKs (FIGS. 2B, 2C, FIGS. 5C, 5D, FIG. 22). Significantly, inhibitors of the “secondary” RTK that mediated ligand-driven rescue in the various tested models had little or no effect as single agent treatments in these cell lines, indicating that the kinase-addicted cells are not initially dependent on multiple different RTKs in the absence of available ligand. Similarly, RTK ligand stimulation had little or no effect on cell proliferation in the absence of kinase inhibitors (FIG. 1C and FIG. 2B).

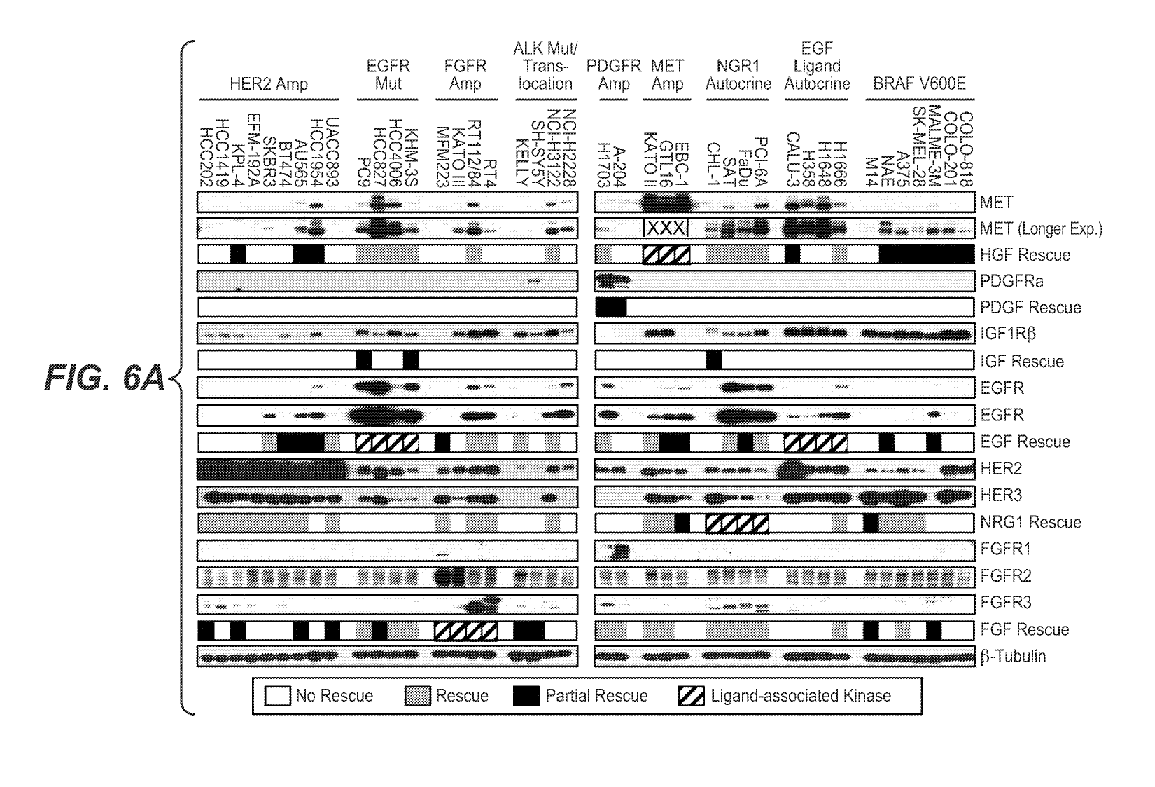

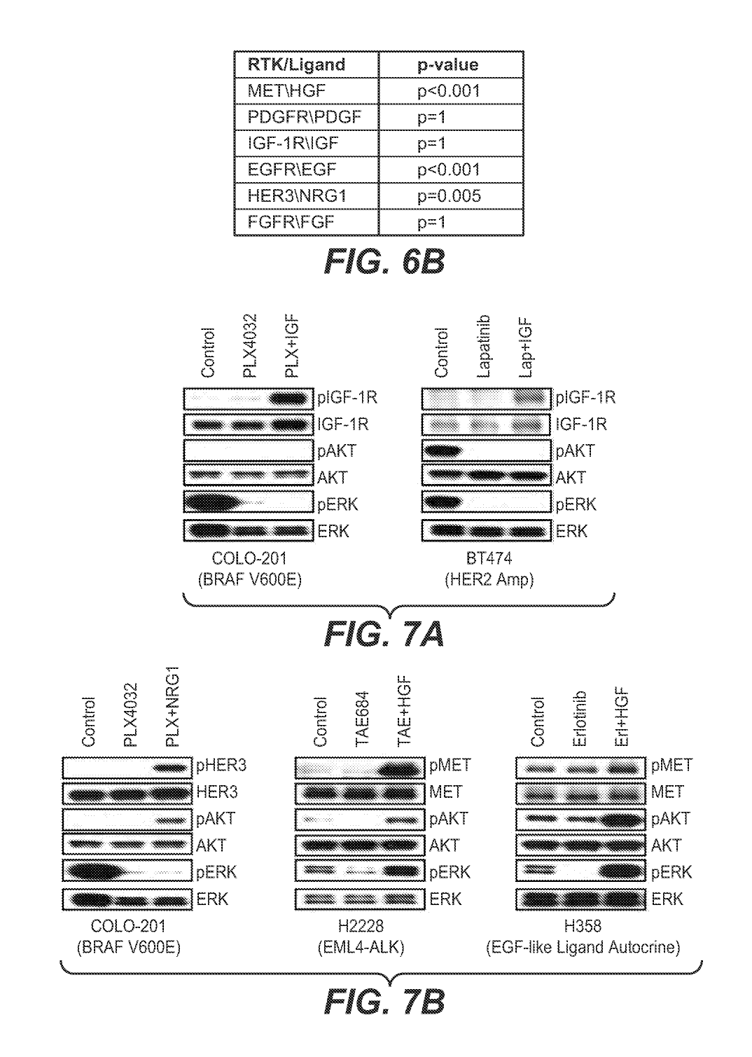

Analysis of baseline RTK expression across the cell line panel confirmed that all of these kinase-dependent cancer cells express multiple RTKs, suggesting that many cancer cells are “primed” to receive survival signals from extracellular ligands. Notably, ligand-induced rescue was well correlated with the expression of certain RTKs in some cases (e.g., MET/HGF, EGFR/EGF and HERS/NRG1) (p<0.01; FIGS. 6A, 6B), suggesting that the RTK profile of tumors prior to treatment could inform an optimal treatment strategy that anticipates the need to co-target two or more kinases that might contribute to cancer cell survival, depending on the availability of corresponding ligands in the tumor microenvironment.

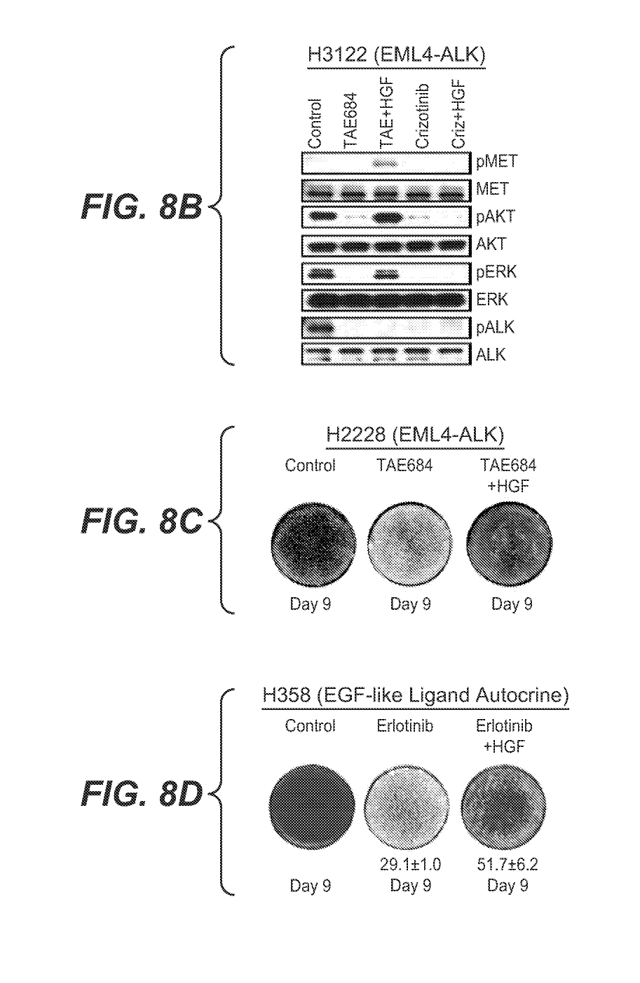

In some cases, ligands were unable to rescue cells from drug sensitivity despite the expression of the ligand-associated RTK. We identified two different biochemical scenarios associated with a failure of ligand-induced rescue in this context (FIGS. 7A-7C). In a few cases, the RTK ligand was able to activate its receptor, as evidenced by RTK phosphorylation; however, consequent downstream signalling via PI3K or MAPK was not observed. This was seen, for example, in the COLO-201 and BT474 cell lines upon treatment with IGF (FIG. 7A). In other cases, the RTK ligand activated its receptor as well as at least one downstream survival effector; however, that was not sufficient to rescue cells from kinase inhibition. This was observed, for example, with H2228 and H358 cells upon exposure to HGF, or with COLO-201 cells upon exposure to NRG1 (FIG. 7B). However, H2228 and H358 cells are “rescued” by HGF following longer-term treatment, possibly implicating the existence of a subpopulation of cells that are capable of responding to HGF and which might be selected over time in the presence of an inhibitory kinase, as elaborated below (FIGS. 8C, 8D).

The cell line analysis yielded several findings with potentially important clinical implications. For example, one of two tested NSCLC cell lines harbouring an ALK-associated chromosomal translocation (NCI-H3122), and exhibiting ALK kinase addiction, could be efficiently rescued from ALK inhibition by brief exposure to HGF (FIGS. 8A-8D). In these cells, where the HGF receptor MET is expressed, HGF promotes ERK and AKT activation even in the presence of the ALK-selective inhibitor TAE684. Significantly, however, survival of these cells was efficiently suppressed even in the presence of HGF by treatment with crizotinib, a dual ALK/MET kinase inhibitor that has recently demonstrated impressive clinical activity in ALK-translocated NSCLCs¹². In light of the observed capacity of these cells to respond to HGF, the relatively durable clinical responses observed in many of the ALK-translocated NSCLC patients might be attributed in part to the dual inhibitory nature of crizotinib, which can effectively suppress both ALK- and MET-mediated survival signals. Interestingly, the second ALK-translocated NSCLC line, NCI-H2228 also expresses detectable MET, but was not rescued from ALK inhibition by HGF at the tested 72 hour time-point. However, HGF treatment was able to re-activate AKT and ERK activity in the presence of TAE684 (FIG. 7B), and longer-term TAE684 treatment in the presence of HGF prevented acquired resistance to TAE684 in these cells (FIG. 8C). This finding is reminiscent of the previously described pre-existing MET-expressing tumor cell subpopulation has been shown to be present in some EGFR mutant NSCLC patients¹³.

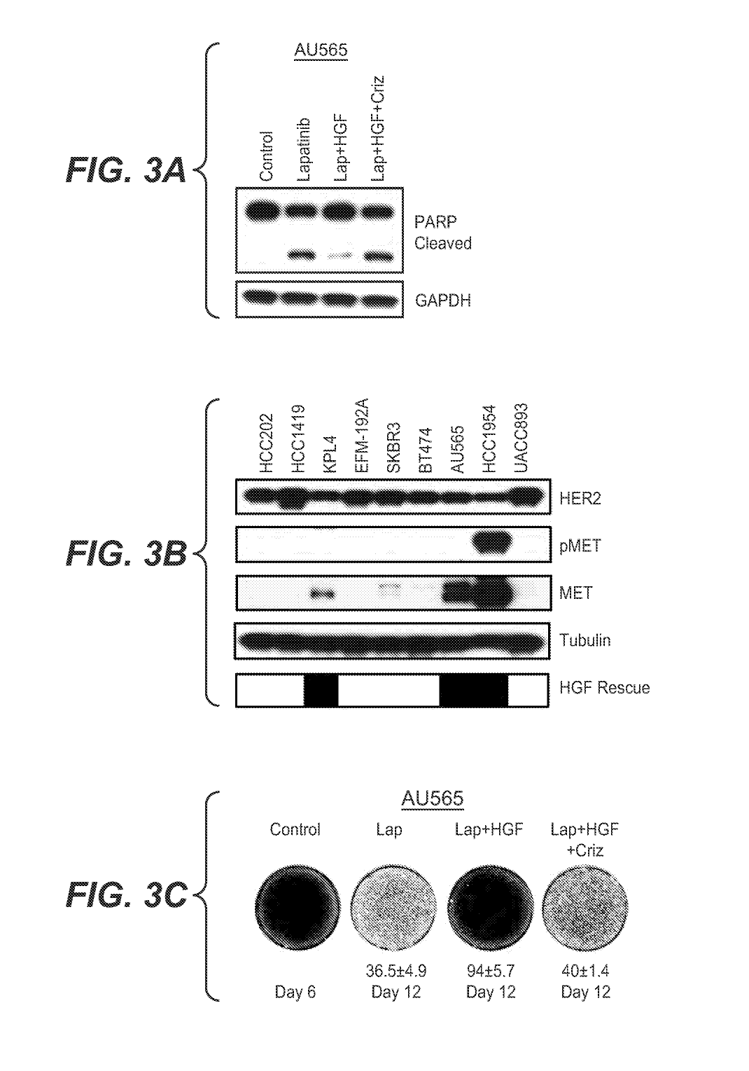

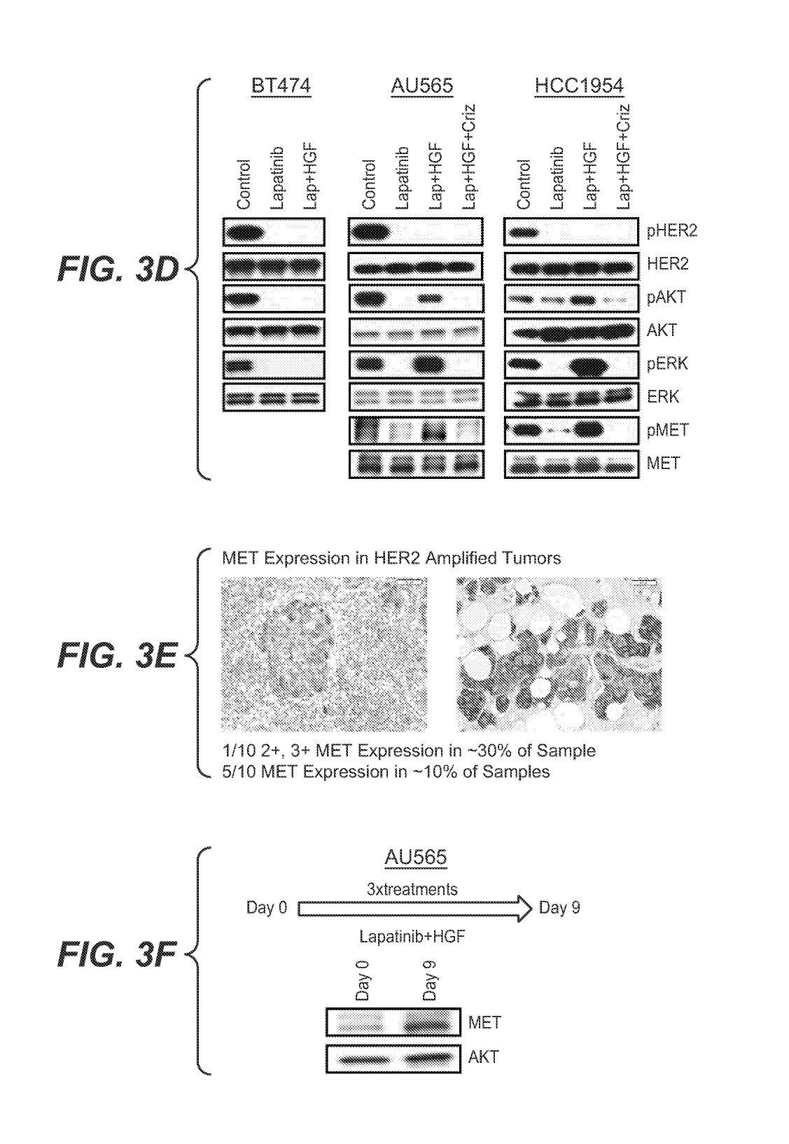

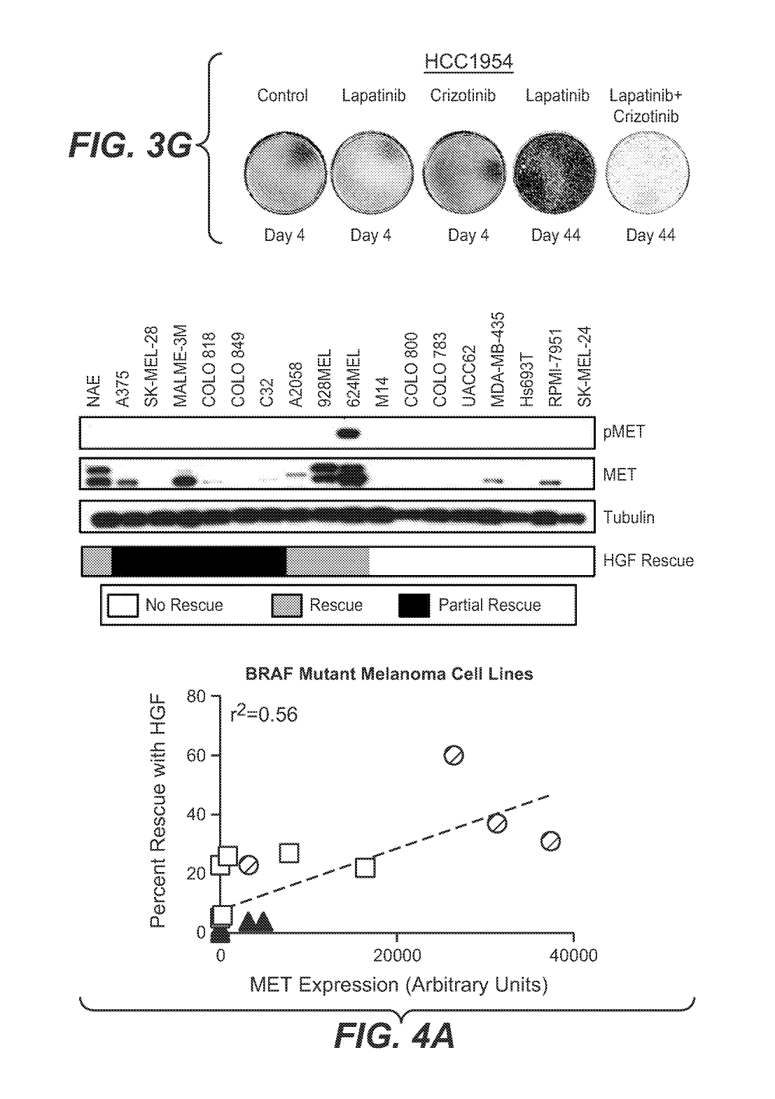

The ability of HGF to rescue 3 of 9 tested HER2-amplified breast cancer cell lines from growth inhibition by the HER2 kinase inhibitor lapatinib was also unexpected (FIG. 3A). These 3 cell lines all express MET, and expression was well correlated with the ability of HGF to attenuate lapatinib response (FIG. 3B). As in the NCI-H228 cell line, longer-term co-treatment (12 days) of the partially HGF-rescued AU565 MET-expressing cells revealed that HGF rapidly promoted resistance to lapatinib, presumably by driving selection of a subpopulation of MET-expressing cells (FIG. 3C and FIG. 9B). Indeed, 9-day lapatinib and HGF co-treatment of AU565 cells yielded a population of cells with increased MET expression, suggesting that HGF exposure selected for a subpopulation of MET-expressing cells (FIG. 3F). Biochemical analysis indicated that HGF re-activated PI3K and MAPK signalling pathways specifically in MET-positive, but not in MET-negative cells (FIG. 3D).

We next determined if HER2-positive primary breast tumors detectably express MET protein (FIG. 3E). Out of ten samples analysed, one sample exhibited moderate and high MET expression in ˜30% of tumor cells and five samples displayed MET expression in approximately 10% of tumor cells. One HER2 amplified breast cancer cell line (HCC1954) displayed elevated phospho-MET in the absence of exogenous HGF, implicating an autocrine mechanism (FIG. 3B), and MET kinase inhibition in these cells delayed the emergence of lapatinib resistance (FIG. 3G). Collectively, these results suggest that MET-expressing HER2-positive breast tumors could potentially evade HER2 kinase inhibition by engaging MET in a subpopulation of “primed” tumor cells, resulting in resistance to targeted therapy, and that this switch to MET dependency may be driven by the availability of HGF. Consistent with this possibility, SKBR3 and AU565 cells were derived from the same patient, highlighting the likely heterogeneity of MET expression within patient tumors. We also found that 8 of the 9 tested HER2-amplified breast cell lines could be rescued from lapatinib sensitivity by exposure to the HER3 ligand NRG1, implicating a potentially important role for NRG1 expression in the tumor microenvironment in the variable response to HER2-targeted treatments (FIG. 23).

Another observation with immediate potential clinical implications was the unexpected finding that HGF exposure significantly attenuated the response to the BRAF kinase inhibitor PLX4032 in several tested BRAF mutant PLX4032-sensitive melanoma and colorectal cell lines. PLX4032 recently demonstrated remarkable clinical efficacy in BRAF mutant melanoma, leading to its recent approval for clinical use¹⁴.

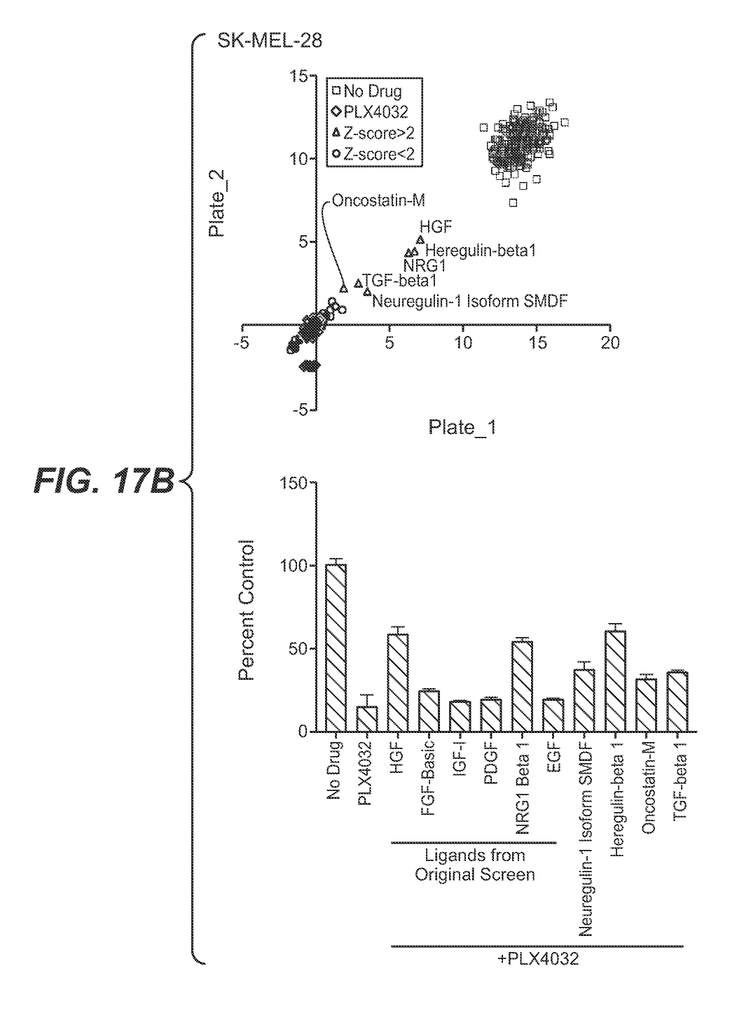

To determine the potential role for growth factors and other cytokines other than HGF to similarly impact PLX4032 sensitivity, we compared the sensitivity of SK-MEL-28 cells to PLX4032 in the presence of each of 446 different recombinant purified secreted factors. This analysis revealed that a very small number of factors, including HGF, could attenuate PLX4032 sensitivity (FIG. 17B).

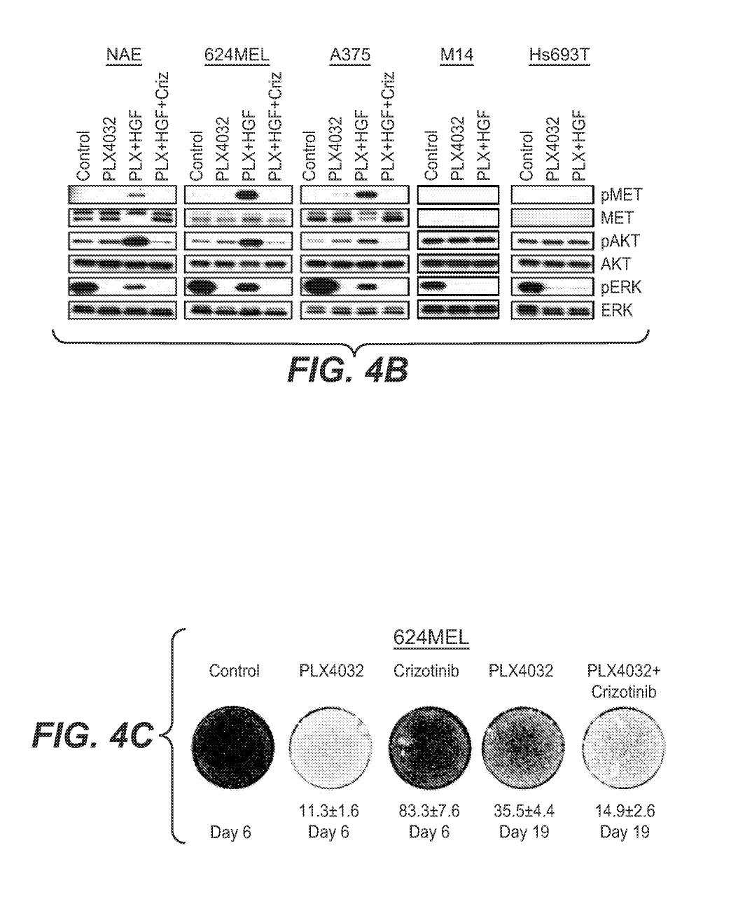

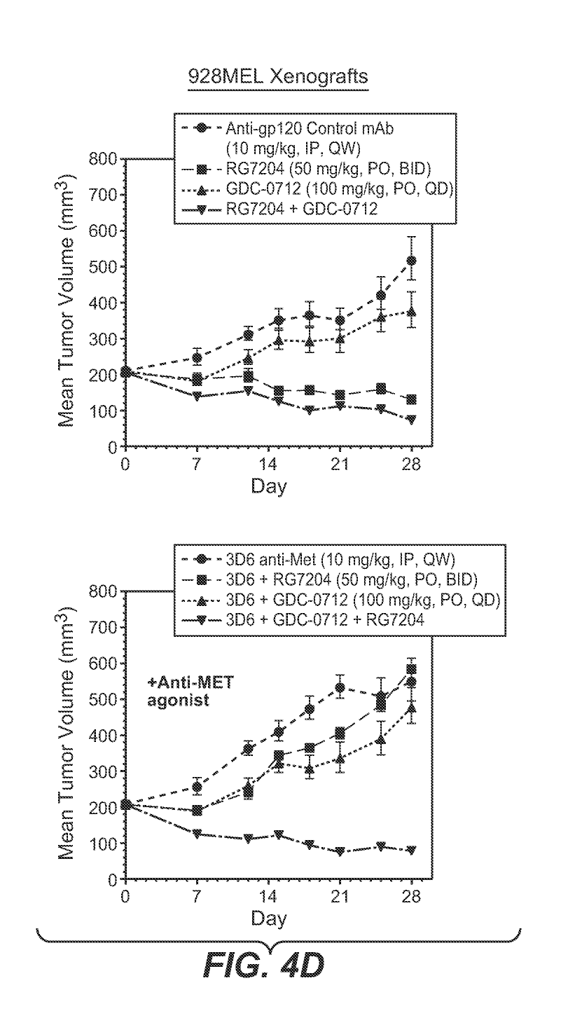

We examined an additional twelve BRAF mutant melanoma cell lines to explore the potentially broader role of HGF-MET signalling in the response to PLX4032 (FIG. 4A). HGF significantly attenuated PLX4032 sensitivity in 5 of the 12 lines. Eight of ten HGF-rescued cell lines displayed detectable MET expression, whereas MET was undetectable or barely detectable in the non-recued cells. Notably MET expression was inversely correlated with the PLX4032 sensitivity in the HGF-rescuable cell lines, and HGF could re-activate MAPK signalling in cell lines that were rescued by HGF, but not in the MET-negative HOF-non-rescued cells (FIG. 4B). As anticipated, survival rescue by HGF was reversed when MET was inhibited by crizotinib (FIG. 4B and FIG. 9A). One BRAF mutant cell line (624MEL) displayed elevated phospho-MET in the absence of exogenous HGF, consistent with an autocrine mechanism (FIG. 4A), and MET kinase inhibition in these cells delayed the emergence of PLX4032 resistance (FIG. 4C).

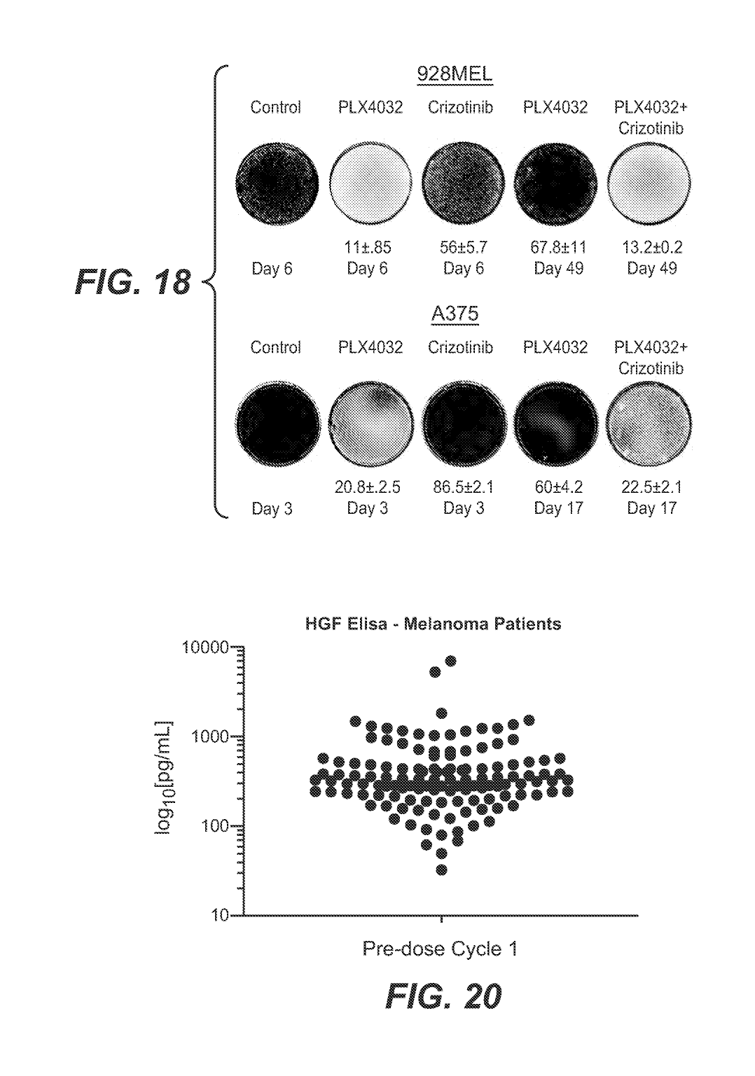

Crizotinib co-treatment also prevented resistance to PLX4032 in two cell lines (A375 and 928MEL) with undetectable phospho-MET, further supporting a potential role for HGF-activated MET in mediating resistance to PLX4032 (FIG. 18).

To verify a potential role for HGF-MET signalling in resistance to BRAF inhibition in vivo, we performed a xenograft study with BRAF mutant 928MEL melanoma cells. Significantly, activation of MET in these tumors using the agonistic antibody 3D6 abrogated the growth-suppressive effects of PLX4032 (FIG. 4D). The relevance of MET activation by 3D6 in attenuating response to PLX4032 was demonstrated by co-treating with a MET small molecule kinase inhibitor. Collectively, these results suggest that MET kinase, via HGF activation, could contribute to the clinical response to PLX4032 in a subset of BRAF mutant melanomas.

The overall findings highlight the extensive nature of signal cross-talk among RTKs that can be co-expressed in most tumor cells, and the potentially broad role of RTK ligands in contributing to innate and acquired resistance to selective kinase inhibitors as cancer therapeutics. Such ligands could be produced by tumor cells themselves to drive autocrine survival mechanisms or could be produced by tumor stroma to impact drug response in tumor cells via paracrine effects on survival signalling^{15,16.}

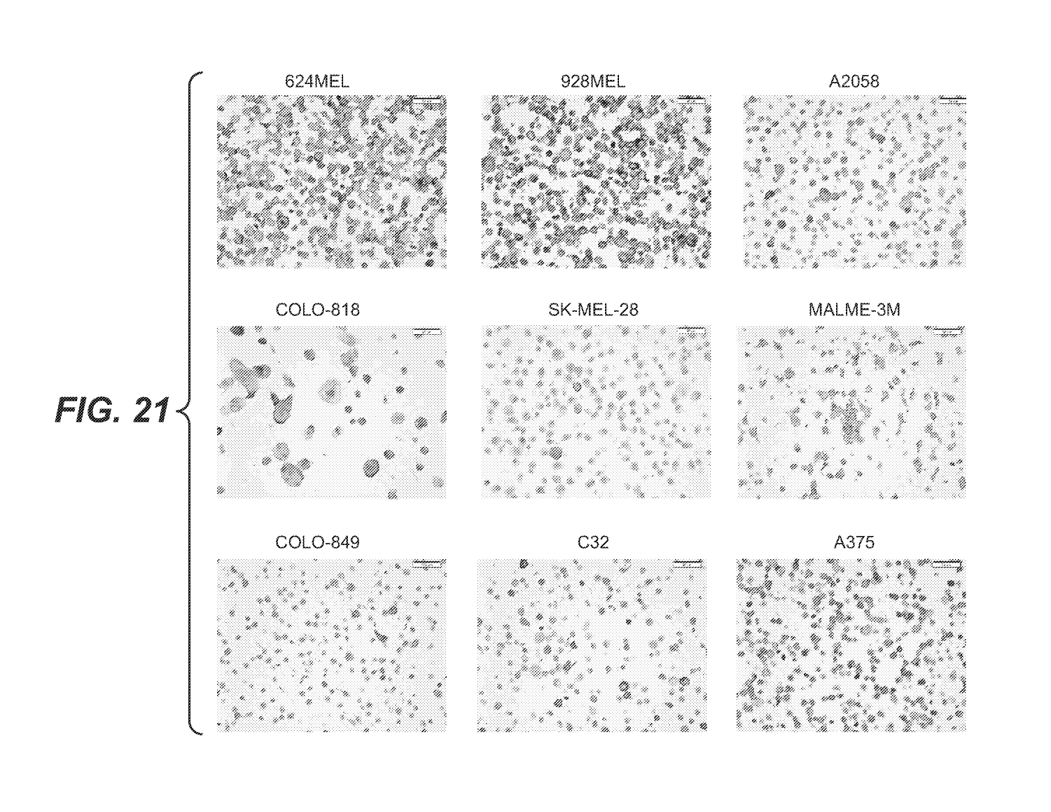

The increasingly appreciated heterogeneity of human tumors significantly complicates the elucidation of drug resistance mechanisms¹⁷⁻¹⁹. In the context of our findings that highlight a potentially broad role for RTK ligands, we imagine distinct mechanisms by which such heterogeneity could contribute to acquired resistance. Thus, it is possible that a subpopulation of tumor cells is present prior to therapy that is capable of responding to a survival-promoting RTK ligand, and that this subpopulation is expanded through the selective pressure of drug treatment if such a ligand becomes available within the tumor microenvironment. Indeed, IHC analysis of MET expression in the BRAF mutant melanoma cells revealed a heterogeneous population of cells (FIG. 21). In the case of EGFR mutant NSCLC, a subpopulation of MET-driven tumor cells can emerge upon exposure to HGF during treatment with EGFR kinase inhibitors¹³. Notably, activation of multiple RTK's has been reported in glioblastoma, and suppression of pro-survival signals and cell death was only observed following co-targeting multiple activated receptors (Stommel, J. M. et al. Science 318, 287-290, doi:10.1126/science.1142946 (2007)). It is also possible that a subpopulation of tumor cells is selected by virtue of acquiring the ability to produce an RTK ligand. In a variety of pre-clinical models of acquired resistance to selective kinase inhibitors, the observed resistance mechanism involved a “switch” to a new RTK dependency²⁰⁻²⁵, which in some cases could be attributed to an increase in production of an RTK ligand. Such increased ligand production could potentially be achieved either by mutational or epigenetic mechanisms.

While genomic biomarkers, such as BRAF and EGFR mutations, have been critical in identifying patients most likely to benefit from therapy, there is an as yet unexplained wide range of initial clinical response to kinase inhibitory drugs among such patients—both in terms of magnitude and duration of response^{12,14}. The potential role for RTK ligands secreted by tumor cells, expressed in the tumor microenvironment, or even provided systemically, has been largely unexplored thus far. As tumor-derived cell lines have proven to be a robust model for capturing the genotype-associated sensitivity to selective kinase inhibitors in mutationally-defined subsets^{7,8}, the findings from this matrix analysis support a potentially broad role for RTK ligands in the overall clinical benefit from such therapies, and provide a foundation for the use of biomarkers based on the expression of RTKs and their associated ligands to inform treatment strategies that anticipate both innate and acquired resistance mechanisms associated with redundant survival signalling through key effectors common to many widely expressed RTKs.

### Example 2

### Rescue Results of Various PTK Ligands in Cells with BRAF V600E

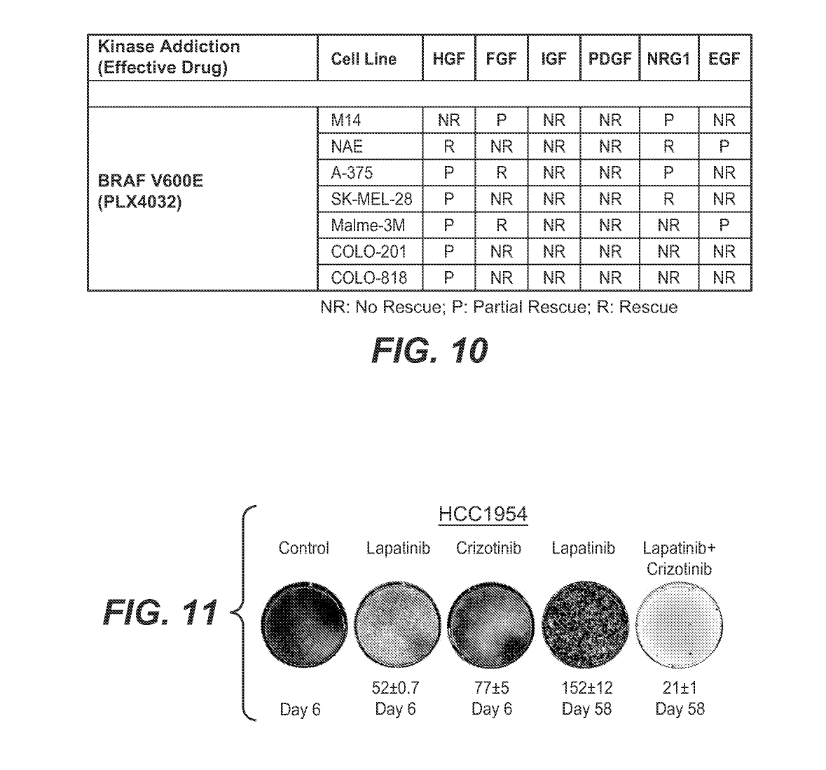

The method used herein is similar to what is described in Example 1. We examined the effects of 6 different RTK ligands (HGF, EGF, FGF, PDGF, NRG1, IGF) on drug response (PLX4032) in cells with BRAF V600E. FIG. 10 shows the rescue results by various PTK ligands in the cells treated with PLX4032.

### Example 3

### Effects of MET Kinase Inhibition in Delaying Lapatinib Resistance

The method used herein is similar to what is described in Example 1. The effects of MET kinase inhibition to delay lapatinib resistance in HCC 1954 cells were examined. HCC1954 HER2 amplified breast cancer cells were treated with lapatinib (5 μM) and/or crizotinib (1 μM) and stained with Syto 60. FIG. 11 shows that MET kinase inhibition in HCC1954 cells delayed the emergence of lapatinib resistance.

### Example 4

### Role of HGF-MET Signaling in Cell Response to PLX4032

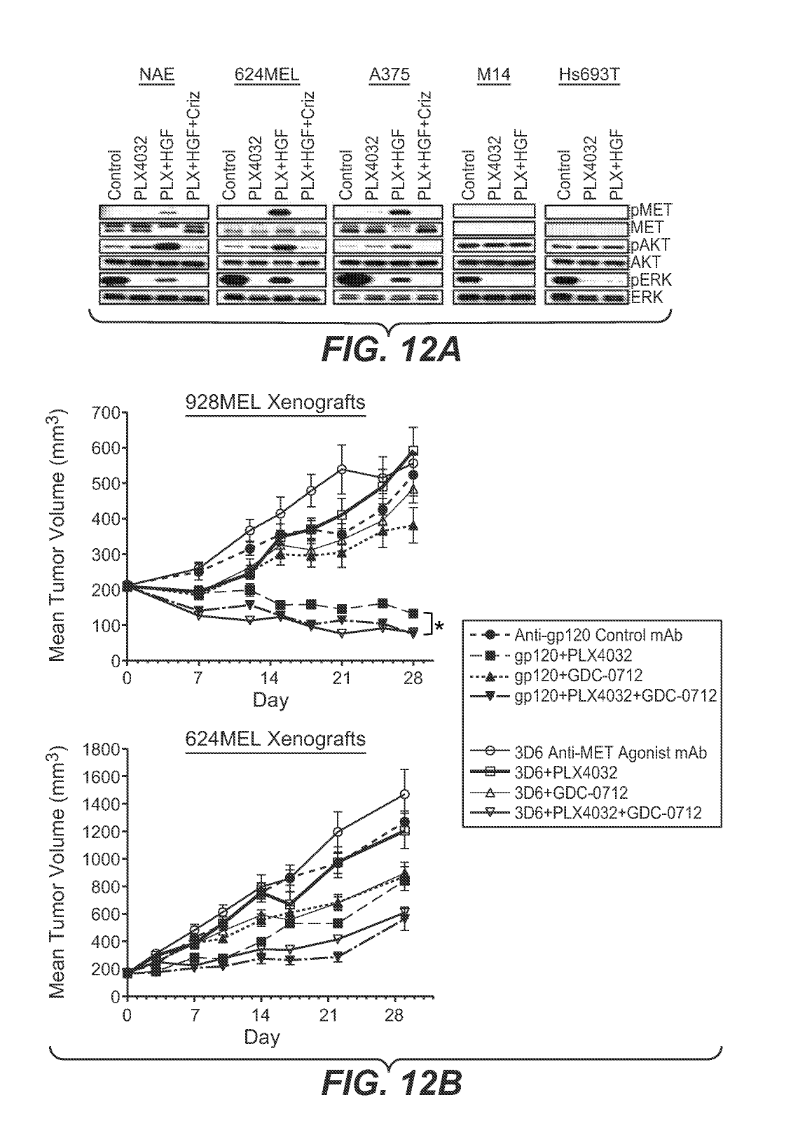

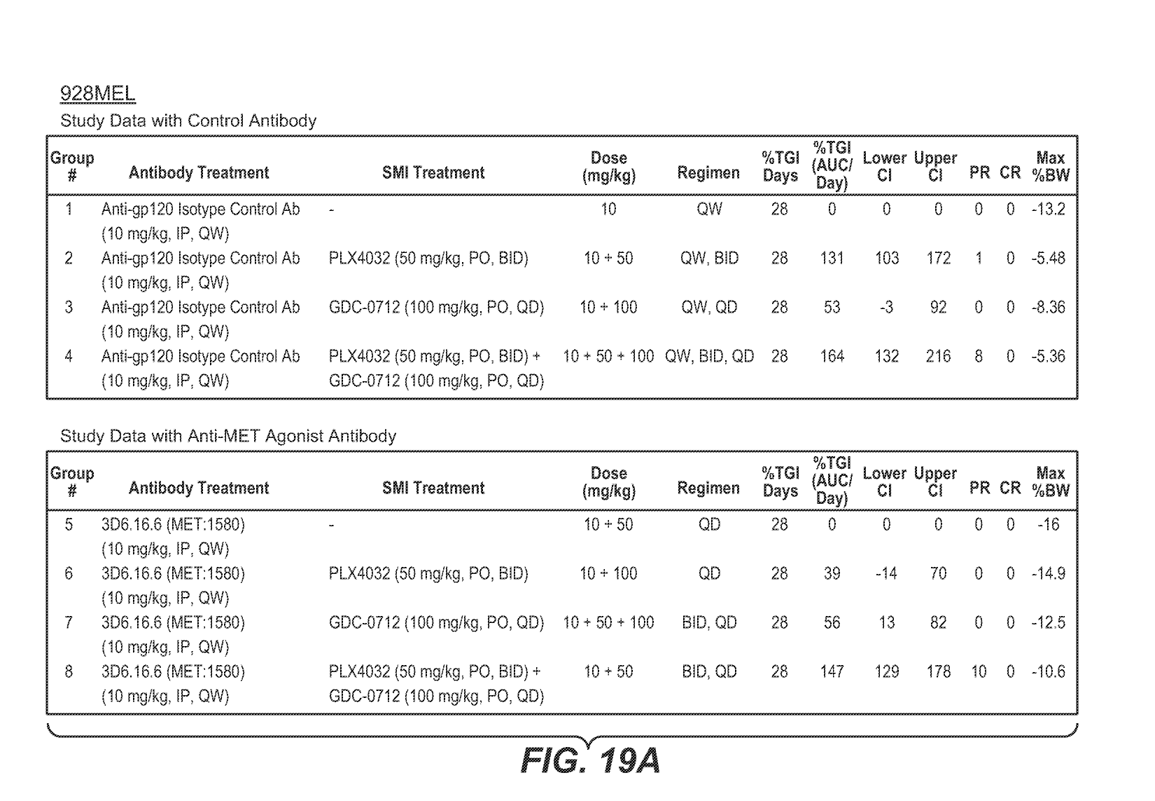

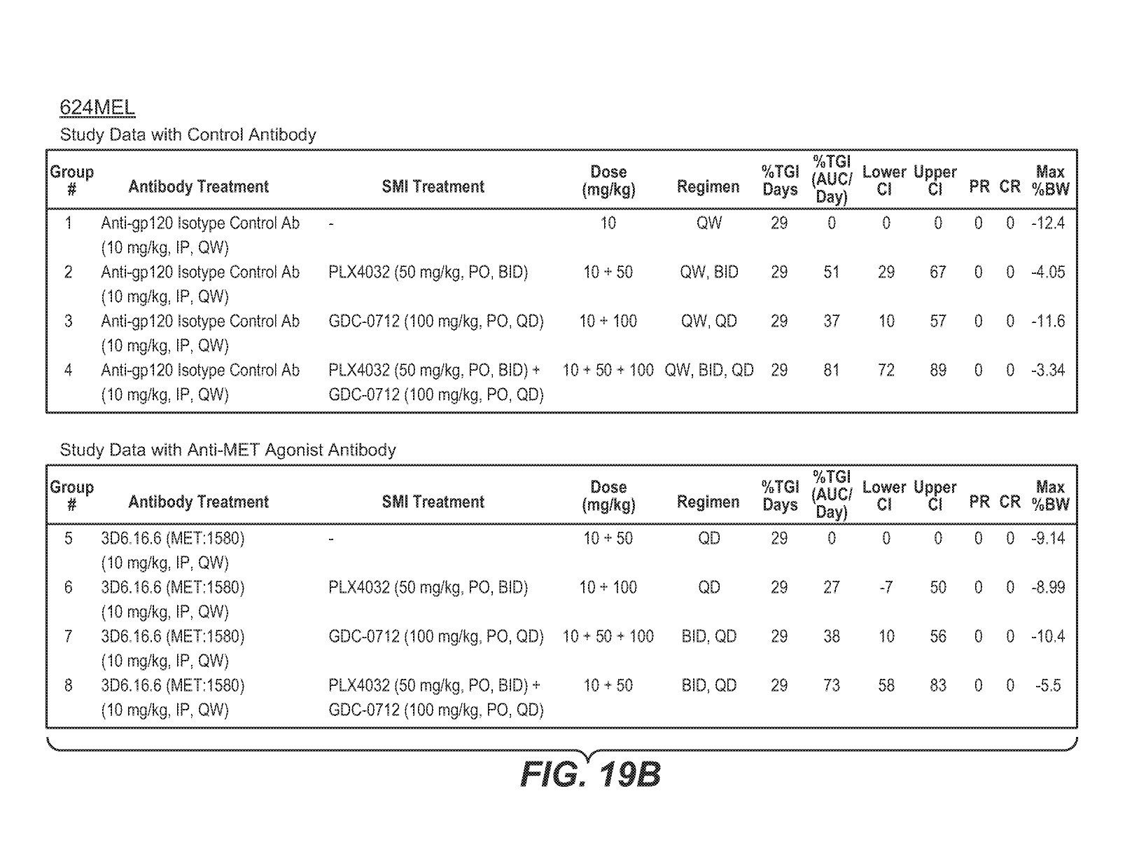

The method used herein is similar to what is described in Example 1. We examined the role of HGF-MET signalling in cell response to PLX4032. We observed that HGF could re-activate MAPK signalling in cell lines that were rescued by HGF, but not in the MET-negative HOF-non-rescued cells (FIG. 4A and FIG. 12A). To verify a potential role for HGF-MET signalling in resistance to BRAF inhibition in vivo, we performed xenograft studies with BRAF mutant 928MEL and 624MEL melanoma cells. Significantly, activation of MET in these tumors using the MET-agonist antibody 3D6 strongly abrogated the growth-suppressive effects of PLX4032 (FIG. 12B). The relevance of MET activation by 3D6 in attenuating response to PLX4032 was verified by co-treating with a MET small molecule kinase inhibitor. Similar to the in vitro findings, we observed that inhibiting MET kinase activity had a greater effect on tumor regression in PLX4032-treated xenografts, with more partial responses observed (928MEL: 1 vs 8; FIG. 12B and FIGS. 19A, 19B). Collectively, these results suggest that MET kinase, via HGF activation, could contribute to the clinical response to PLX4032 in a subset of BRAF mutant melanomas.

### Example 5

### Role for HGF-MET Signaling in Clinical Context

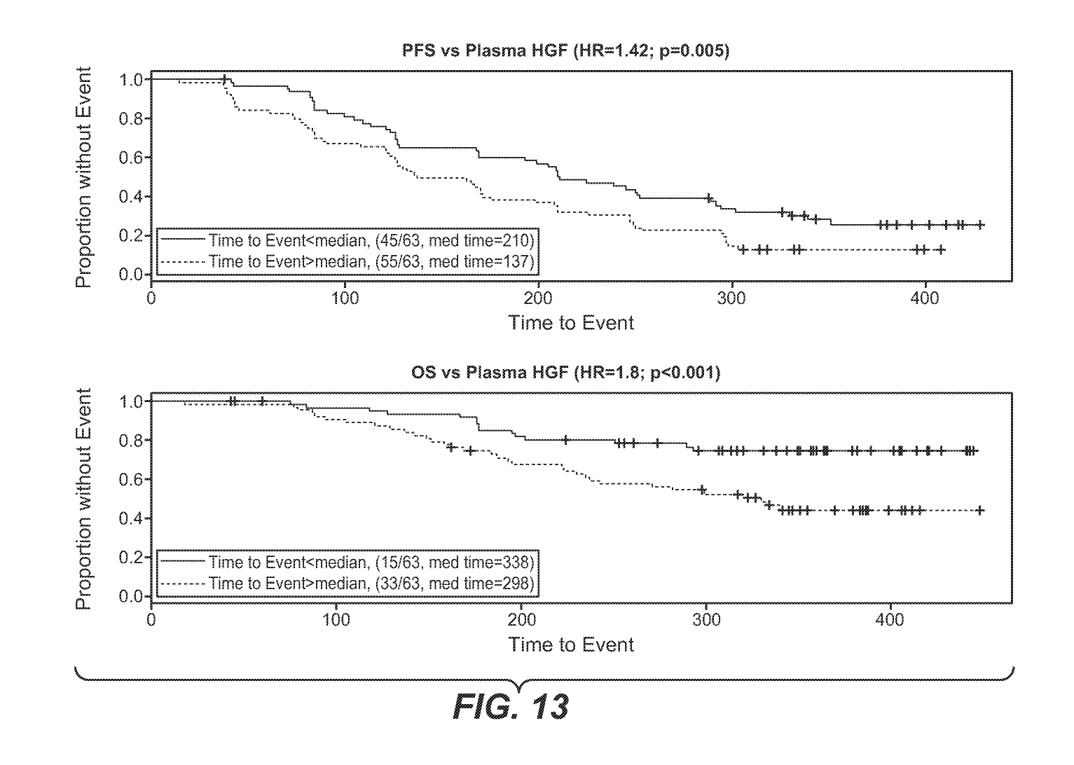

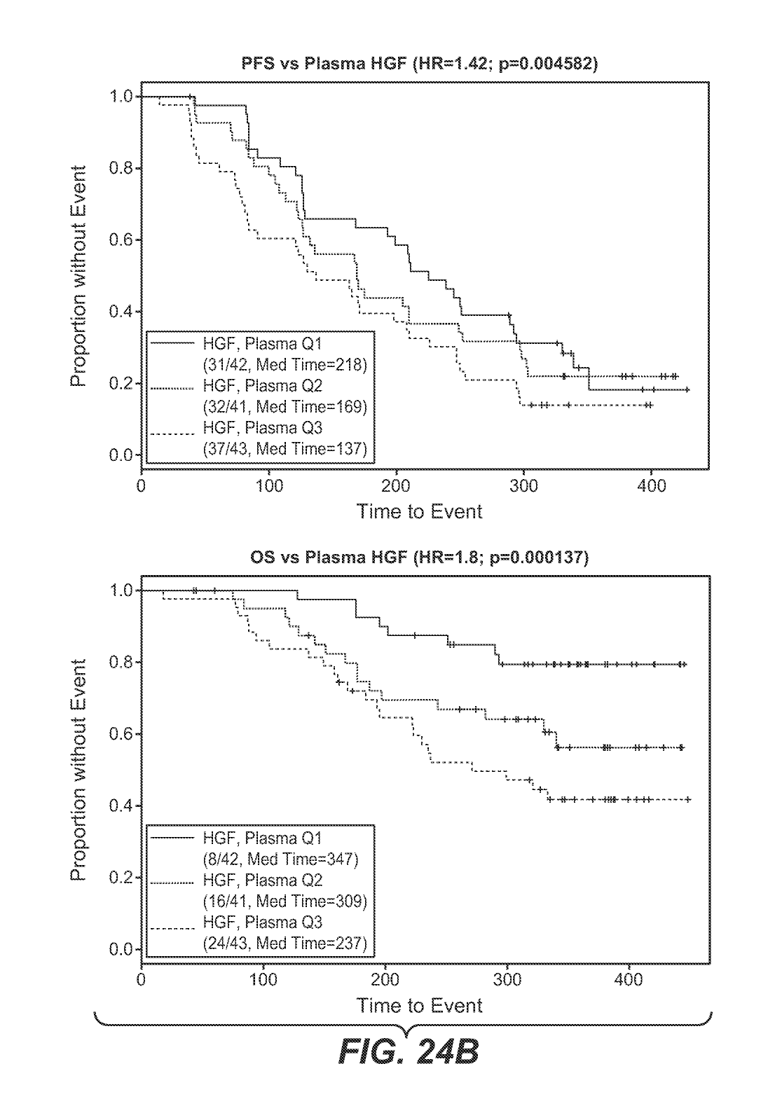

The method used herein is similar to what is described in Example 1. To examine a potential role for HGF-MET signalling in clinical context, we tested the hypothesis that circulating HGF in BRAF mutant melanoma patients could contribute to clinical outcome. Thus, pre-treatment plasma HGF levels were measured from 126 of the 132 metastatic melanoma patients that were enrolled onto the BRIM2 clinical trial (BRAF mutant metastatic melanoma patients treated with PLX4032). HGF levels ranged from 33 pg/mL to 7200 pg/mL with a median level of 334 pg/mL (FIG. 20). PLX4032-treated patients with HGF levels above the median demonstrated substantially reduced progression-free survival (p=0.005) and overall survival (p<0.001) than patients with HGF levels below median (FIG. 13). Increased HGF was associated with worse outcome as measured by progression free survival (PFS, hazard ratio is 1.42 and p<0.005) and overall survival (OS, hazard ratio is 1.8 and p<0.001). Segregating patients into tertiles revealed a continuous relationship between HGF level and outcome, rather than a threshold effect (FIG. 24B). These studies implicate HGF-MET signalling in disease progression and overall survival, and possibly the clinical response to BRAF inhibition in BRAF mutant melanoma.

### Example 6

### Ligand-Induced Rescue in Cells

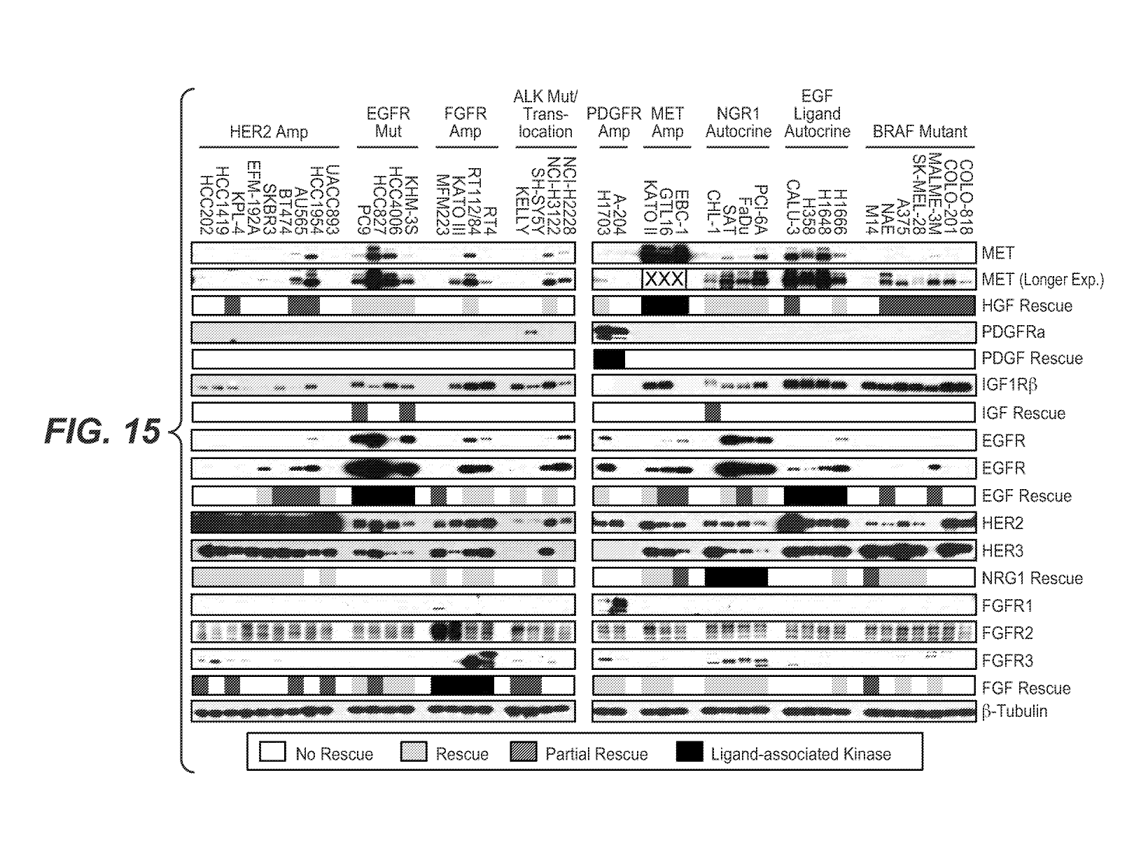

The method used herein is similar to what is described in Example 1. We analysed expression of RTKs and the ligand-induced rescue in cells. The results are shown in FIG. 15. The ligand-induced rescue was well correlated with the expression of certain RTKs in some cases (e.g., MET/HGF, EGFR/EGF and HER3/NRG1) (p<0.01; FIG. 15), suggesting that the RTK profile of tumors prior to treatment could inform an optimal treatment strategy that anticipates the need to co-target two or more kinases that might contribute to cancer cell survival, depending on the availability of corresponding ligands in the tumor microenvironment.

### Example 7

### Effects of HGF in Preventing Acquired Resistance to TAE684

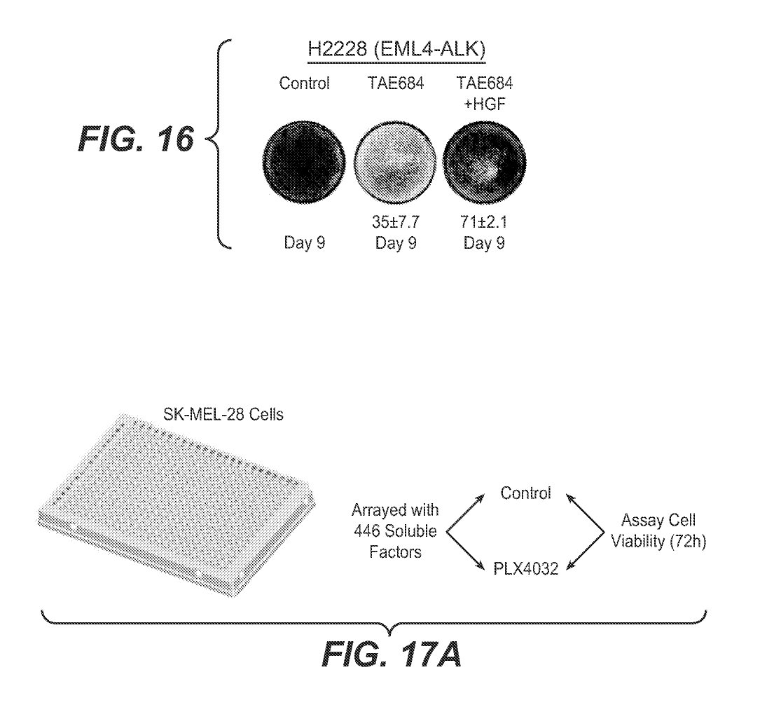

The method used herein is similar to what is described in Example 1. We examined the effect of HGF in H2228 cells treated with TAE 684. FIG. 16 shows that longer-term TAE684 treatment in the presence of HGF prevented acquired resistance to TAE684 in these cells.

### PARTIAL REFERENCE LIST

1 Sharma, S. V. & Settleman, J. Oncogene addiction: setting the stage for molecularly targeted cancer therapy. Genes Dev 21, 3214-3231. doi:10.1101/gad.1609907 (2007).
2 Sequist, L. V. et al. Genotypic and histological evolution of lung cancers acquiring resistance to EGFR inhibitors. Sci Transl Med 3, 75ra26, doi:10.1126/scitranslmed.3002003 (2011).
3 Garrett, J. T. & Arteaga. C. L. Resistance to HER2-directed antibodies and tyrosine kinase inhibitors: mechanisms and clinical implications. Cancer Biol Ther 11, 793-800 (2011).
4 Engelman, J. A. & Settleman, J. Acquired resistance to tyrosine kinase inhibitors during cancer therapy. Curr Opin Genet Dev 18, 73-79, doi:10.1016/j.gde.2008.01.004 (2008).
Moritz, A. et al. Akt-RSK-S6 kinase signaling networks activated by oncogenic receptor tyrosine kinases. Sci Signal 3, ra64, doi:10.1126/scisignal.2000998 (2010).
6 Zhang, W. & Huang, P. Cancer-stromal interactions: role in cell survival, metabolism and drug sensitivity. Cancer Biol Ther 11, 150-156 (2011).
7 McDermott, U. et al. Identification of genotype-correlated sensitivity to selective kinase inhibitors by using high-throughput tumor cell line profiling. Proc Natl Acad Sci USA 104, 19936-19941, doi:10.1073/pnas.0707498104 (2007).
8 Wilson, T. R., Lee, D. Y., Berry, L., Shames. D. S. & Settleman, J. Neuregulin-1-Mediated Autocrine Signaling Underlies Sensitivity to HER2 Kinase Inhibitors in a Subset of Human Cancers. Cancer Cell 20, 158-172, doi:10.1016/j.ccr.2011.07.011 (2011).
9 Yonesaka, K. et al. Autocrine production of amphiregulin predicts sensitivity to both gefitinib and cetuximab in EGFR wild-type cancers. Clin Cancer Res 14, 6963-6973, doi:10.1158/1078-0432.CCR-08-0957 (2008).
Mueller, M. M. & Fusenig, N. E. Friends or foes—bipolar effects of the tumor stroma in cancer. Nat Rev Cancer 4, 839-849, doi:10.1038/nrc1477 (2004).
11 Grant, S., Qiao, L. & Dent, P. Roles of ERBB family receptor tyrosine kinases, and downstream signaling pathways, in the control of cell growth and survival. Front Biosci 7, d376-389 (2002).
12 Kwak, E. L. et al. Anaplastic lymphoma kinase inhibition in non-small-cell lung cancer. N Engl J Med 363, 1693-1703, doi:10.1056/NEJMoa006448 (2010).
13 Turke, A. B. et al. Preexistence and clonal selection of MET amplification in EGFR mutant NSCLC. Cancer Cell 17, 77-88, doi:10.1016/j.ccr.2009.11.022 (2010).
14 Chapman, P. B. et al. Improved survival with vemurafenib in melanoma with BRAF V600E mutation. N Engl J Med 364, 2507-2516, doi:10.1056/NEJMoa1103782 (2011).
Gilbert, L. A. & Hemann, M. T. DNA damage-mediated induction of a chemoresistant niche. Cell 143, 355-366, doi:10.1016/j.cell.2010.09.043 (2010).
16 Liang, K. et al. Recombinant human erythropoietin antagonizes trastuzumab treatment of breast cancer cells via Jak2-mediated Src activation and PTEN inactivation. Cancer Cell 18, 423-435, doi:10.1016/j.ccr.2010.10.025 (2010).
17 Quintana, E. et al. Phenotypic heterogeneity among tumorigenic melanoma cells from patients that is reversible and not hierarchically organized. Cancer Cell 18, 510-523, doi:10.1016/j.ccr.2010.10.012 (2010).
18 Calbo, J. et al. A functional role for tumor cell heterogeneity in a mouse model of small cell lung cancer. Cancer Cell 19, 244-256, doi:10.1016/j.ccr.2010.12.021 (2011).
19 Sharma, S. V. et al. A chromatin-mediated reversible drug-tolerant state in cancer cell subpopulations. Cell 141, 69-80, doi:10.1016/j.cell.2010.02.027 (2010).
20 Nazarian, R. et al. Melanomas acquire resistance to B-RAF(V600E) inhibition by RTK or N-RAS upregulation. Nature 468, 973-977, doi:10.1038/nature09626 (2010).
21 Guix, M. et al. Acquired resistance to EGFR tyrosine kinase inhibitors in cancer cells is mediated by loss of IGF-binding proteins. J Clin Invest 118, 2609-2619, doi:10.1172/JCI34588 (2008).
22 McDermott, U., Pusapati, R. V., Christensen, J. G., Gray, N. S. & Settleman, J. Acquired resistance of non-small cell lung cancer cells to MET kinase inhibition is mediated by a switch to epidermal growth factor receptor dependency. Cancer Res 70, 1625-1634, doi:10.1158/0008-5472.CAN-09-3620 (2010).
23 Johannessen. C. M. et al. COT drives resistance to RAF inhibition through MAP kinase pathway reactivation. Nature 468, 968-972, doi:10.1038/nature09627 (2010).
24 Liu, L et al. Novel mechanism of lapatinib resistance in HER2-positive breast tumor cells: activation of AXL. Cancer Res 69, 6871-6878, doi:10.1158/0008-5472.CAN-08-4490 (2009).
Engelman, J. A. et al. MET amplification leads to gefitinib resistance in lung cancer by activating ERBB3 signaling. Science 316, 1039-1043, doi:10.1126/science.1141478 (2007).
26 Stommel. J. M. et al. Coactivation of receptor tyrosine kinase affects the response of tumor cells to targeted therapies. Science 318, 287-290, doi:10.1126/science.1142946 (2007).
26 Liederer, B. M. et al. Preclinical absorption, distribution, metabolism, excretion, and pharmacokinetic-pharmacodynamic modelling of N-(4-(3-((3S,4R)-1-ethyl-3-fluoropiperidine-4-ylamino)-1H-pyrazolo[3,4-b]pyridin-4-yloxy)-3-fluorophenyl)-2-(4-fluorophenyl)-3-oxo-2,3-dihydropyridazine-4-carboxamide, a novel MET kinase inhibitor. Xenobiotica 41, 327-339, doi:10.3109/00498254.2010.542500 (2011).
27 Catenacci, D. V. T. et al. Durable Complete Response of Metastatic Gastric Cancer with Anti-Met Therapy Followed by Resistance at Recurrence. Cancer Discovery 1, 573-579, doi:10.1158/2159-8290 (2011).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1: A method for treating a patient with cancer comprising administering to the patient an effective amount of B-raf antagonist and c-met antagonist.

2: The method of claim 1, wherein the patient has an increased likelihood of developing resistance to B-raf antagonist.

3. (canceled)

4. (canceled)

5: The method of claim 1, wherein the cancer is a B-raf antagonist resistant cancer.

6. (canceled)

7. (canceled)

8: The method of claim 1, wherein the patient's cancer has been shown to express B-raf biomarker.

9. (canceled)

10. (canceled)

11: The method of claim 8, wherein mutant B-raf biomarker expression in the patient's cancer is determined using a method comprising (a) performing one or more of gene expression profiling, PCR hybridization assay, in situ hybridization, 5′ nuclease assay mutation detection assay, RNA-seq, microarray analysis, SAGE, MassARRAY technique, or FISH on a sample and (b) determining expression of mutant B-raf biomarker in the sample.

12: The method of claim 11, wherein mutant B-raf biomarker expression in the patient's cancer is determined using a method comprising (a) performing PCR on genomic DNA extracted from a patient cancer sample and (b) determining expression of mutant B-raf biomarker in the sample.

13: The method of claim 1, wherein the patient's cancer has been shown to express c-met biomarker.

14: The method of claim 13, wherein c-met biomarker is a polypeptide.

15: The method of claim 14, wherein c-met biomarker expression is determined using immunohistochemistry (IHC).

16: The method of claim 15, wherein c-met biomarker expression is determined by determining expression of hepatocyte growth factor (HGF).

17. (canceled)

18. (canceled)

19: The method of claim 1, wherein the c-met antagonist is an antagonist anti-c-met antibody.

20: The method of claim 1, wherein the c-met antagonist is one or more of onartuzumab, crizotinib, tivantinib, carbozantinib, MGCD-265, ficlatuzumab, humanized TAK-701, rilotumumab, foretinib, h224G11, DN-30, MK-2461, E7050, MK-8033, PF-4217903, AMG208, JNJ-38877605, EMD1204831, INC-280, LY-2801653, SGX-126, RP1040, LY2801653, BAY-853474, and/or LA480.

21: The method of claim 1, wherein the B-raf antagonist is one or more of sorafenib, PLX4720, PLX-3603, GSK2118436, GDC-0879, N-(3-(5-(4-chlorophenyl)-1H-pyrrolo[2,3-b]pyridine-3-carbonyl)-2,4-difluorophenyl)propane-1-sulfonamide, vemurafenib, GSK 2118436, RAF265 (Novartis), XL281, ARQ736, BAY73-4506.

22. (canceled)

23. (canceled)

24: The method of claim 1, wherein the B-raf antagonist and the c-met antagonist are administered simultaneously.

25: The method of claim 1, wherein the B-raf antagonist and the c-met antagonist are administered sequentially.

26. (canceled)

27. (canceled)

28: The method of claim 1, further comprising administering at least one additional treatment to said subject.

29: The method of claim 1, wherein the cancer is melanoma, colorectal, ovarian, breast or papillary thyroid.

30. (canceled)

31: The method of claim 1, wherein the cancer is resistant to B-raf antagonist.

32: The method of claim 1, wherein the patient has not been previously treated with B-raf antagonist.

33. (canceled)

34: A method for identifying a patient having cancer as a candidate for treatment with a B-raf antagonist and a c-met antagonist, comprising (a) determining that the patient's cancer expresses c-met biomarker; and (b) identifying the patient as a candidate for treatment with a B-raf antagonist and a c-met antagonist.

35: A method for identifying a patient having cancer as at risk of developing resistance to a B-raf antagonist, comprising (a) determining that the patient's cancer expresses c-met biomarker; and (b) identifying the patient as at risk of developing resistance to a B-raf antagonist

36: The method of claim 34, wherein subsequent to steps (a) and (b), the patient is treated with an effective amount of a c-met antagonist and a B-raf antagonist.

37: A method of determining therapeutic efficacy of a B-raf antagonist for treating cancer in a patient comprising determining the presence of c-met biomarker and/or B-raf biomarker in a sample obtained from said patient by immunoassay, elisa, hybridization assay, PCR, 5′ nuclease assay, IHC, and/or RT-PCR, and selecting the patient for treatment with a B-raf antagonist.

38: The method of claim 37, further comprising selecting the patient for treatment with a c-met antagonist.

39: The method of claim 38, further comprising treating the patient with an effective amount of B-raf antagonist and c-met antagonist.

40. (canceled)

41: A kit comprising a c-met antagonist and a B-raf antagonist.

42: The kit of claim 41, further comprising instructions for a method for treating a melanoma patient comprising administering an effective amount of a c-met antagonist and B-raf antagonist to the patient.

43. (canceled)


SEQUENCE LISTING (13 sequences)

SEQ ID NO: 1 - protein, 10 aa, Artificial Sequence
  Description of Artificial Sequence Synthetic peptide
GYTFTSYWLH

SEQ ID NO: 2 - protein, 18 aa, Artificial Sequence
  Description of Artificial Sequence Synthetic peptide
GMIDPSNSDTRFNPNFKD

SEQ ID NO: 3 - protein, 12 aa, Artificial Sequence
  Description of Artificial Sequence Synthetic peptide
ATYRSYVTPLDY

SEQ ID NO: 4 - protein, 17 aa, Artificial Sequence
  Description of Artificial Sequence Synthetic peptide
KSSQSLLYTSSQKNYLA

SEQ ID NO: 5 - protein, 7 aa, Artificial Sequence
  Description of Artificial Sequence Synthetic peptide
WASTRES

SEQ ID NO: 6 - protein, 9 aa, Artificial Sequence
  Description of Artificial Sequence Synthetic peptide
QQYYAYPWT

SEQ ID NO: 7 - protein, 119 aa, Artificial Sequence [sequence omitted]

SEQ ID NO: 8 - protein, 114 aa, Artificial Sequence
  Description of Artificial Sequence Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 9 - protein, 222 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 222 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 449 aa, Artificial Sequence
  Description of Artificial Sequence Synthetic polypeptide
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 220 aa, Artificial Sequence
  Description of Artificial Sequence Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK